SEQUENCE LISTING (20 sequences)

SEQ ID NO: 1 - protein, 5 aa, Mus musculus
DYAMH

SEQ ID NO: 2 - protein, 17 aa, Mus musculus
LISIYSDHTNYNQKFQG

SEQ ID NO: 3 - protein, 12 aa, Mus musculus
NNYGNYGWYFDV

SEQ ID NO: 4 - protein, 10 aa, Mus musculus
SASSSVSYMH

SEQ ID NO: 5 - protein, 7 aa, Mus musculus
DTSKLAS

SEQ ID NO: 6 - protein, 9 aa, Mus musculus
FQGSGYPYT

SEQ ID NO: 7 - protein, 121 aa, Artificial Sequence
  Artificially Synthesized Sequence
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 8 - protein, 106 aa, Artificial Sequence
  Artificially Synthesized Sequence
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 9 - protein, 122 aa, Mus musculus
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 10 - protein, 122 aa, Artificial Sequence
  Artificially Synthesized Sequence
QVQLVQSGSELKKPGASVKVSCKASGYTFTDYSMHWVRQAPGQGLKWMGWINTETGEPTYADDFKGAFVFSLDTSVSTAYLQISSLKAEDTAVYYCANPYYDYVSYYAMDYWGQGTTVTVSS

SEQ ID NO: 11 - protein, 107 aa, Mus musculus
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 12 - protein, 107 aa, Artificial Sequence
  Artificially Synthesized Sequence
DIQMTQSPSSLSASVGDRVTITCKASQDVSTAVAIYQQKPGKAPKLLIYSASYLYTGVPSRFSGSGSGTDFTFTISSLQPEDIATYYCQQHYSTPRTFGQGTKLEIK

SEQ ID NO: 13 - protein, 116 aa, Artificial Sequence
  Artificially Synthesized Sequence
EVQLVESGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 14 - protein, 111 aa, Artificial Sequence
  Artificially Synthesized Sequence
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 15 - protein, 10 aa, Mus musculus
GFTFSDYWMD

SEQ ID NO: 16 - protein, 17 aa, Mus musculus
NIDEDGSITEYSPFVKG

SEQ ID NO: 17 - protein, 8 aa, Mus musculus
WGRFGFDS

SEQ ID NO: 18 - protein, 15 aa, Mus musculus
KSSQSLLSGSFNYLT

SEQ ID NO: 19 - protein, 7 aa, Mus musculus
YASTRHT

SEQ ID NO: 20 - protein, 9 aa, Mus musculus
HHHYNAPPT

(19) United States
(12) Patent Application Publication
(10) Pub. No.: US 2020/0062735 A1
(43) Pub. Date: Feb. 27, 2020

(54) HETEROCYCLIC AMIDES AS KINASE INHIBITORS

(71) Applicant: GlaxoSmithKline Intellectual Property Development Limited, Brentford, Middlesex (GB)

(72) Inventors: Jill Marinis ANBARI, Collegeville, PA (US); John J. BERTIN, Collegeville, PA (US); Jae U. JEONG, Collegeville, PA (US)

(21) Appl. No.: 16/488,625

(22) PCT Filed: Feb. 23, 2018
(86) PCT No.: PCT/IB2018/051163

§ 371 Date: Aug. 26, 2019

Related U.S. Application Data
(60) Provisional application No. 62/464,016, filed on Feb. 27, 2017.
(60) Provisional application No. 62/585,216, filed on Nov. 13, 2017.

(51) Int. Cl. C07D 403/12; A61P 35/02; A61P 35/04

(52) U.S. Cl. CPC C07D 403/12; A61K 45/06; A61P 35/04; A61P 35/02

(57) ABSTRACT

Disclosed is a combination of a RIP1 kinase inhibitor compound and at least one other therapeutically active agent for use in the treatment of a RIP1 kinase mediated disease or disorder; particularly disclosed is a combination of a RIP1 kinase inhibitor compound and at least one other therapeutically active agent, wherein the at least one other therapeutically active agent is an immuno-modulator, for use in the treatment of cancer.

### FIELD OF THE INVENTION

The present invention relates to heterocyclic amides that inhibit RIP1 kinase and methods of making and using the same. The present invention also relates to combinations of RIP1 kinase inhibitors and at least one other therapeutically active agent and methods of using said combination in the treatment of cancer.

### BACKGROUND OF THE INVENTION

Receptor-interacting protein-1 (RIP1) kinase, originally referred to as RIP, is a TKL family serine/threonine protein kinase involved in innate immune signaling. RIP1 kinase is a RHIM domain containing protein, with an N-terminal kinase domain and a C-terminal death domain (Trends Biochem. Sci. 30, 151-159 (2005)). The death domain of RIP1 mediates interaction with other death domain containing proteins including Fas and TNFR-1 (Cell 81, 513-523 (1995)), TRAIL-R1 and TRAIL-R2 (Immunity 7, 821-830 (1997)) and TRADD (Immunity 4, 387-396, (1996)), while the RHIM domain is crucial for binding other RHIM domain containing proteins such as TRIF (Nat Immunol. 5, 503-507 (2004)), DAI (EMBO Rep. 10, 916-922 (2009)) and RIP3 (J. Biol. Chem. 274, 16871-16875 (1999); Curr. Biol. 9, 539-542 (1999)) and exerts many of its effects through these interactions. RIP1 is a central regulator of cell signaling, and is involved in mediating both pro-survival and programmed cell death pathways which will be discussed below.

The role for RIP1 in cell signaling has been assessed under various conditions [including TLR3 (Nat Immunol. 5, 503-507 (2004)), TLR4 (J. Biol. Chem. 280, 36560-36566 (2005)), TRAIL (FAS (J. Biol. Chem. 279, 7925-7933 (2004))], but is best understood in the context of mediating signals downstream of the death receptor TNFR1 (Cell 114, 181-190 (2003)). Engagement of the TNFR by TNF leads to its oligomerization, and the recruitment of multiple proteins, including linear K63-linked polyubiquitinated RIP1 (Mol. Cell 22, 245-257 (2006)), TRAF2/5 (J. Mol. Biol. 396, 528-539 (2010)), TRADD (Nat. Immunol. 9, 1037-1046 (2008)) and cIAPs (Proc. Natl. Acad. Sci. USA. 105, 11778-11783 (2008)), to the cytoplasmic tail of the receptor. This complex which is dependent on RIP1 as a scaffolding protein (i.e. kinase independent), termed complex I, provides a platform for pro-survival signaling through the activation of the NFκB and MAP kinases pathways (Sci. Signal. 115, re4 (2010)). Alternatively, binding of TNF to its receptor under conditions promoting the deubiquitination of RIP1 (by proteins such as A20 and CYLD or inhibition of the cIAPs) results in receptor internalization and the formation of complex II or DISC (death-inducing signaling complex) (Cell Death Dis. 2, e230 (2011)). Formation of the DISC, which contains RIP1, TRADD, FADD and caspase 8, results in the activation of caspase 8 and the onset of programmed apoptotic cell death also in a RIP1 kinase independent fashion (FEBS J 278, 877-887 (2012)). Apoptosis is largely a quiescent form of cell death, and is involved in routine processes such as development and cellular homeostasis.

Under conditions where the DISC forms and RIP3 is expressed, but apoptosis is inhibited (such as FADD/caspase 8 deletion, caspase inhibition or viral infection), a third RIP1 kinase-dependent possibility exists. RIP3 can now enter this complex, become phosphorylated by RIP1 and initiate a caspase-independent programmed necrotic cell death through the activation of MLKL and PGAMS (Cell 148, 213-227 (2012)); (Cell 148, 228-243 (2012)); (Proc. Natl. Acad. Sci. USA. 109, 5322-5327 (2012)). As opposed to apoptosis, programmed necrosis (not to be confused with passive necrosis which is not programmed) results in the release of danger associated molecular patterns (DAMPs) from the cell. These DAMPs are capable of providing a “danger signal” to surrounding cells and tissues, eliciting proinflammatory responses including inflammasome activation, cytokine production and cellular recruitment (Nat. Rev. Immunol 8, 279-289 (2008)).

Dysregulation of RIP1 kinase-mediated programmed cell death has been linked to various inflammatory diseases, as demonstrated by use of the RIP3 knockout mouse (where RIP1-mediated programmed necrosis is completely blocked) and by Necrostatin-1 (a tool inhibitor of RIP1 kinase activity with poor oral bioavailability). The RIP3 knockout mouse has been shown to be protective in inflammatory bowel disease (including Ulcerative colitis and Crohn's disease) (Nature 477, 330-334 (2011)), Psoriasis (Immunity 35, 572-582 (2011)), retinal-detachment-induced photoreceptor necrosis (PNAS 107, 21695-21700 (2010)), retinitis pigmentosa (Proc. Natl. Acad. Sci., 109:36, 14598-14603 (2012)), cerulein-induced acute pancreatits (Cell 137, 1100-1111 (2009)) and Sepsis/systemic inflammatory response syndrome (SIRS) (Immunity 35, 908-918 (2011)). Necrostatin-1 has been shown to be effective in alleviating ischemic brain injury (Nat. Chem. Biol. 1, 112-119 (2005)), retinal ischemia/reperfusion injury (J. Neurosci. Res. 88, 1569-1576 (2010)), Huntington's disease (Cell Death Dis. 2 e115 (2011)), renal ischemia reperfusion injury (Kidney Int. 81, 751-761 (2012)), cisplatin induced kidney injury (Ren. Fail. 34, 373-377 (2012)) and traumatic brain injury (Neurochem. Res. 37, 1849-1858 (2012)). Other diseases or disorders regulated at least in part by RIP1-dependent apoptosis, necrosis or cytokine production include hematological and solid organ malignancies (Genes Dev. 27: 1640-1649 (2013), Cancer Cell 28, 582-598 2015); pancreatic cancer (Nature 532, 245-249 (2016), Nature 536, 215-218 (2016)), bacterial infections and viral infections (Cell Host & Microbe 15, 23-35 (2014)) (including, but not limited to, tuberculosis and influenza (Cell 153, 1-14, (2013)) and Lysosomal storage diseases (particularly, Gaucher Disease, Nature Medicine Advance Online Publication, 19 Jan. 2014, doi:10.1038/nm.3449). Inflammation is known to be a contributing factor in the pathogenesis of diabetes and obesity (Chen. et. al., International Journal of Endocrinology (2015)). Blocking the actions of TNF at the TNF receptor has been shown to improve glucose homeostasis in animals and humans (Stagakis et al., Arthritis Research & Therapy (2012)). Inhibition of RIP1 has been implicated in protection against the Rd10 mouse model of human retinitis pigmentosa (RP) (Y. Murakami et al., PNAS 109(36):14598-14603 (2012)). Inhibition of RIP1 has been implicated in protection against the experimental autoimmune encephalomyelitis (EAE) mouse model of human Multiple Sclerosis (MS) (D. Ofengeim et al. Cell Reports 10(11):1836-1849, (2015)). RIP1 is a serine/threonine protein kinase closely aligned with RIP3 in that their co-association results in necroptosis (Shutinoski, B. et al. Cell Death Differ. 23, 1628-1637, doi:10.1038/cdd.2016.51 (2016)). However, RIP1 additionally drives NF-κB and MAP kinase signaling in response to inflammatory stimuli independently of its association with RIP3 (Meylan, E. et al. Nat. Immunol. 5, 503-507, doi:10.1038/ni1061 (2004) and Ofengeim, D. & Yuan, J. Nat. Rev. Mol. Cell Biol. 14, 727-736, doi:10.1038/nrm3683 (2013)). RIP1 is also a putative master upstream regulator of TLR signaling (Ofengeim, D. & Yuan, J.). Hence, RIP1 may have pleiotropic influences on suppressive macrophage polarization in cancer.

A potent, selective, small molecule inhibitor of RIP1 kinase activity would block RIP1-dependent cellular necrosis and might block suppressive macrophage polarization in cancer and thereby provide a therapeutic benefit in diseases or events associated with DAMPs, cell death, and/or inflammation as well as be useful in combination treatment with immuno-modulators. Thus, there is a need for new combination therapies of RIP1 kinase inhibitors with other therapeutically active agents, in particular, immuno-modulators.

### SUMMARY OF THE INVENTION

This invention is directed to a method of treating a RIP1 kinase-mediated disease or disorder which comprises administering a therapeutically effective amount of a compound that inhibits RIP1 kinase, particularly a compound described herein, to a patient (a human or other mammal, particularly, a human) in need thereof. The invention is still further directed a method of treating a RIP1 kinase-mediated disease or disorder which comprises administering a therapeutically effective amount of a compound that inhibits RIP1 kinase and at least one other therapeutically active agent to a human in need thereof.

In particular, this invention is directed to combinations of RIP1 kinase inhibitors with at least one other therapeutically active agent and methods of using said combination in the treatment of cancer. This invention is more specifically directed to a combination of RIP1 kinase inhibitor and an immuno-modulator and methods of using said combination in the treatment of cancer.

This invention is also directed to a compound that inhibits RIP1 kinase for use with at least one other therapeutically active agent in the treatment of a RIP1 kinase-mediated disease or disorder. The invention is further directed to combinations of a compound that inhibits RIP1 kinase, particularly a compound described herein, for use in therapy, in particular, in the treatment of a RIP1 kinase-mediated disease or disorder, more particularly, in the treatment of cancer.

The invention is still further directed to the use of a combination of a compound that inhibits RIP1 kinase, particularly a compound described herein, in the manufacture of a medicament for the treatment of a RIP1 kinase-mediated disease or disorder, more particularly, in the treatment of cancer.

In the combination of this invention, a compound disclosed in WO2014/125444 that inhibits RIP1 kinase is a compound of Formula (I):

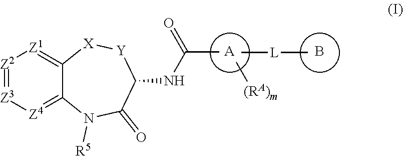

wherein:
X is O, S, SO, SO₂, NH, CO, CH₂, CF₂, CH(CH₃), CH(OH), or N(CH₃);
Y is CH₂ or CH₂CH₂;
Z¹ is N, CH or CR¹;
Z² is CH or CR²;
Z³ is N, CH or CR³;
Z⁴ is CH or CR⁴;
R¹ is fluoro or methyl;
one of R² and R³ is halogen, cyano, (C₁-C₆)alkyl, halo(C₁-C₄)alkyl, (C₁-C₆)alkoxy, halo(C₁-C₄)alkoxy, hydroxyl, B(OH)₂, —COOH, halo(C₁-C₄)alkylC(OH)₂—, (C₁-C₄)alkoxy(C₁-C₄)alkoxy, (C₁-C₄)alkylSO₂-, (C₁-C₄)alkylSO₂NHC(O)—, (C₁-C₄)alkylC(O)NH—, ((C₁-C₄)alkyl)((C₁-C₄)alkyl)NC(O)—, (C₁-C₄)alkylOC(O)—, (C₁-C₄)alkylC(O)N(C₁-C₄)alkyl)-, (C₁-C₄)alkylNHC(O)—, (C₁-C₄)alkoxy(C₂-C₄)alkylNHC(O)—, (C₁-C₄)alkoxy(C₂-C₄)alkylC(O)NH—, (C₁-C₄)alkoxy(C₂-C₄)alkylNHC(O)NH—, (C₁-C₄)alkylSO₂(C₂-C₄)alkylNHC(O)—, (C₁-C₄)alkylNHC(O)NH—, (C₁-C₄)alkylOC(O)NH—, hydroxy(C₁-C₄)alkylOC(O)NH—, 5-6 membered heterocycloalkyl-C(O)—, 5-6 membered heterocycloalkyl-(C₁-C₄)alkyl-NHC(O)—, 5-6 membered heterocycloalkyl-(C₁-C₄)alkoxy-, 3-6 membered cycloalkyl, 5-6 membered heteroaryl, or 5-6 membered heteroaryl-C(O)NH,
wherein said 3-6 membered cycloalkyl, 5-6 membered heterocycloalkyl and 5-6 membered heteroaryl are optionally substituted by 1 or 2 substituents each independently selected from the group consisting of (C₁-C₄)alkyl and —(C₁-C₄)alkyl-CN;
and the other of R² and R³ is halogen, cyano or (C₁-C₆)alkyl;
R⁴ is fluoro, chloro, methyl or trifluoromethyl;
R⁵ is H or methyl;
A is phenyl, 5-6 membered heteroaryl, or 5-6 membered heterocycloalkyl, wherein the carbonyl moiety and L are substituted 1,3 on ring A;
m is 0 or m is 1 and R^{A} is (C₁-C₄)alkyl; and
L is O, S, NH, N(CH₃), CH₂, CH₂CH₂, CH(CH₃), CHF, CF₂, CH₂O, CH₂N(CH₃), CH₂NH, or CH(OH);
B is an optionally substituted (C₃-C₆)cycloalkyl, phenyl, 5-6 membered heteroaryl, or 5-6 membered heterocycloalkyl;
wherein said (C₃-C₆)cycloalkyl, phenyl, 5-6 membered heteroaryl, or 5-6 membered heterocycloalkyl is unsubstituted or is substituted by one or two substituents each independently selected from halogen, (C₁-C₄)alkyl, halo(C₁-C₄)alkyl, (C₁-C₄)alkoxy, halo(C₁-C₄)alkoxy, nitro, and (C₁-C₄)alkylC(O)—;
or the moiety -L-B is (C₃-C₆)alkyl, (C₃-C₆)alkoxy, halo(C₃-C₆)alkoxy, (C₃-C₆)alkenyl, or (C₃-C₆)alkenyloxy;
or a salt, particularly, a pharmaceutically acceptable salt thereof.

The invention is still further directed to a combination of comprising a compound that inhibits RIP1 kinase, particularly a compound according to Formula (II):

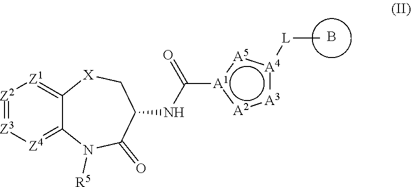

wherein:
X is CH₂ or NH;
Z¹ is CH;
Z² is CH or CR²;
Z³ is CH;
Z⁴ is CH or CR⁴;
R² and R⁴ are each independently selected from chloro or fluoro;
R⁵ is H or methyl;
L is CH₂;
A¹ and A⁴ are C, and A², A³, and A⁵ are each independently selected from N and NH to form a triazolyl ring moiety,
B is a phenyl ring, optionally substituted by fluoro;
or a salt, particularly a pharmaceutically acceptable salt, thereof.

This invention is directed to a method of treating a RIP1 kinase-mediated disease or disorder which comprises administering a therapeutically effective amount of a combination of a compound that inhibits RIP1 kinase, particularly a compound according to Formula (I), or Formula (II), or a salt, particularly a pharmaceutically acceptable salt thereof, to a patient (a human or other mammal, particularly, a human) in need thereof.

This invention is directed to a method of treating a RIP1 kinase-mediated disease or disorder, particularly, a method of treating cancer, which comprises administering a therapeutically effective amount of a combination of a compound that inhibits RIP1 kinase, particularly a compound according to Formula (I), or Formula (II), or a salt, particularly a pharmaceutically acceptable salt thereof, with an immuno-modulator, to a patient (a human or other mammal, particularly, a human) in need thereof.

The invention is further directed to a combination of compound that inhibits RIP1 kinase, particularly a compound according to Formula (I) or Formula (II), or a salt, particularly a pharmaceutically acceptable salt thereof, for use in therapy, in particular, in the treatment of a RIP1 kinase-mediated disease or disorder.

The invention is still further directed to a combination of compound that inhibits RIP1 kinase, particularly a compound according to Formula (I) or Formula (II), or a salt, particularly a pharmaceutically acceptable salt thereof, with an immuno-modulator, for use in therapy, in particular, in the treatment of a RIP1 kinase-mediated disease or disorder, more particularly, in the treatment of cancer.

The invention is still further directed to the use of a combination of a compound that inhibits RIP1 kinase, particularly a compound according to Formula (I), or a salt, particularly a pharmaceutically acceptable salt thereof, in the manufacture of a medicament for the treatment of a RIP1 kinase-mediated disease or disorder.

RIP1 kinase-mediated diseases or disorders are described herein and include inflammatory bowel disease (including Crohn's disease and ulcerative colitis), psoriasis, retinal detachment, retinitis pigmentosa, arthritis (including rheumatoid arthritis, spondyloarthritis, gout, osteoarthritis, and systemic onset juvenile idiopathic arthritis (SoJIA)), transplant rejection, organ transplantation (for donors and recipients), multiple sclerosis, tumor necrosis factor receptor-associated periodic syndrome, multiple organ dysfunction syndrome (MODS), thermal injury/burn, systemic inflammatory response syndrome (SIRS), radiation injury, radiotherapy, chemotherapy, pneumonias, hemorrhagic shock, trauma (including multiple trauma), traumatic brain injury, acute pancreatitis, critical illness (in general), sepsis, septic shock, Stevens-Johnson syndrome, toxic epidermal necrolysis, stroke, heat stroke, stroke-associated pneumonia, Multi-Organ Dysfunction Syndrome (MODS), Acute Respiratory Distress Syndrome (ARDS), intestinal obstruction, liver cirrhosis, surgery, major abdominal operations, abdominal aortic aneurysm repair, large bowel resections, ischemia reperfusion injury (including ischemia reperfusion injury of solid organs, (gut, brain, liver, kidney), and limb ischemia), bowel ischemia (small intestine and large intestine), and cardiac surgery requiring cardio-pulmonary bypass.

The invention is further directed to a method of treating a RIP1 kinase-mediated disease or disorder which comprises administering a therapeutically effective amount of a combination of a compound that inhibits RIP1 kinase to a patient (a human or other mammal, particularly, a human) in need thereof, wherein the RIP1 kinase-mediated disease or disorder is selected from pancreatic cancer, metastatic adenocarcinoma of the pancreas, pancreatic ductal adenocarcinoma, a malignancy of the endocrine cells in the pancreas, hepatocellular carcinoma, mesothelioma, melanoma, colorectal cancer, acute myeloid leukemia, metastasis, glioblastoma, breast cancer, gallbladder cancer, clear cell renal carcinoma, non-small cell lung carcinoma, and radiation induced necrosis. The invention is still further directed to a method of treating a patient (a human or other mammal, particularly, a human) who has undergone solid tumor resection comprising administering a therapeutically effective amount of a combination of a compound that inhibits RIP1 kinase to the patient.

The invention is further directed to a method of treating a RIP1 kinase-mediated disease or disorder which comprises administering a therapeutically effective amount of a compound that inhibits RIP1 kinase in combination with an immuno-modulator to a patient (a human or other mammal, particularly, a human) in need thereof, wherein RIP1 kinase-mediated disease or disorder is selected from pancreatic cancer, metastatic adenocarcinoma of the pancreas, pancreatic ductal adenocarcinoma, a malignancy of the endocrine cells in the pancreas, hepatocellular carcinoma, mesothelioma, melanoma, colorectal cancer, acute myeloid leukemia, metastasis, glioblastoma, breast cancer, gallbladder cancer, clear cell renal carcinoma, non-small cell lung carcinoma, and radiation induced necrosis. The invention is still further directed to a method of treating a patient (a human or other mammal, particularly, a human) who has undergone solid tumor resection comprising administering a therapeutically effective amount of a compound that inhibits RIP1 kinase in combination with an immuno-modulator to the patient.

In addition, this invention is directed to a pharmaceutical composition for the treatment of a RIP1 kinase-mediated disease or disorder, where the composition comprises a compound according to Formula (I) or Formula (II), or a salt, particularly a pharmaceutically acceptable salt, thereof and a pharmaceutically acceptable excipient.

### BRIEF DESCRIPTION OF THE DRAWINGS

FIG. 1A shows the temperature loss over time in mice after oral pre-dosing with the compound of Example 6 or vehicle followed by simultaneous i.v. administration of mouse TNF and zVAD.

FIG. 1B shows the temperature loss in mice 3 hours after oral pre-dosing with the compound of Example 6 or vehicle followed by simultaneous i.v. administration of mouse TNF and zVAD.

FIG. 2A shows subcutaneous pancreatic tumor model with Example 6 alone or in combination with anti-PD1 antibody.

FIG. 2B shows subcutaneous bladder tumor model with Example 6 alone or in combination with anti-PD1 antibody.

FIG. 3A shows the percentage of mice without severe dermatitis over time. After weaning mice received daily in-diet dosing with compound of Example 6 or control diet as indicated and were monitored for development of dermatitis.

FIG. 3B shows the percentage of mice without severe dermatitis over time. Once mice developed clinical signs of dermatitis (about 6 weeks of age), mice received daily in-diet dosing with compound of Example 6 or control diet as indicated and were monitored for development of severe dermatitis.

FIG. 4 shows subcutaneous pancreatic tumor model with Example 6 alone or in combination with ICOS.

### DETAILED DESCRIPTION OF THE INVENTION

It will be appreciated by those skilled in the art that the compounds of this invention, depending on further substitution, may exist in other tautomeric forms. It will be further appreciated by those skilled in the art that any of the RIP1 kinase inhibitor compounds useful in the methods of this invention, may exist in other tautomeric forms. All tautomeric forms of the compounds described herein are intended to be encompassed within the scope of the present invention. It is to be understood that any reference to a named compound or a structurally depicted compound is intended to encompass all tautomers of such compounds and any mixtures of tautomers thereof. It will also be appreciated by those skilled in the art that when A¹ and A⁴ are C, and A², A³, and A⁵ are each independently selected from N and NH, the compounds useful in this invention may exist as triazole tautomers represented by Formulas (I-A), (I-B) and (I-C):

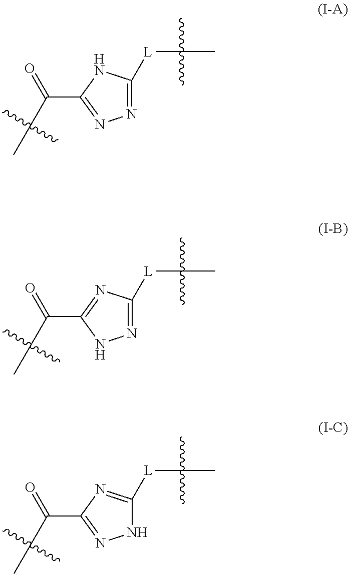

The chemical names provided for the intermediate compounds and/or the compounds useful in this invention described herein may refer to any one of the tautomeric representations of such compounds (in some instances, such alternate names are provided within the experimentals). It is to be understood that any reference to a named compound (an intermediate compound or a compound useful in this invention) or a structurally depicted compound (an intermediate compound or a compound useful in this invention) is intended to encompass all tautomers of such compounds and any mixtures of tautomers thereof.

As used herein, the term “optionally substituted” indicates that the B phenyl group may be unsubstituted, or the phenyl group, may be substituted with one fluoro substituent.

As used herein, the terms “compound(s) of the invention” or “compound(s) of this invention” mean a compound of Formula (I), particularly a compound of any one of Formula (I), as defined herein, in any form, i.e., any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi-hydrates)), and mixtures of various forms.

Accordingly, included within the present invention are the compounds of Formula (I) or Formula (II), particularly, compounds of any one of Formula (I) or Formula (II), as defined herein, in any salt or non-salt form and any physical form thereof, and mixtures of various forms. While such are included within the present invention, it will be understood that the compounds of Formula (I) or Formula (II), particularly, compounds of any one of Formula (I) or Formula (II), as defined herein, in any salt or non-salt form, and in any physical form thereof, may have varying levels of activity, different bioavailabilities and different handling properties for formulation purposes.

In one embodiment of this invention, the compounds of Formula (II) do not include:
(S)-5-benzyl-N-(2-oxo-2,3,4,5-tetrahydro-1H-benzo[b]azepin-3-yl)-4H-1,2,4-triazole-3-carboxamide;
(S)-5-benzyl-N-(1-methyl-2-oxo-2,3,4,5-tetrahydro-1H-benzo[b]azepin-3-yl)-4H-1,2,4-triazole-3-carboxamide;
(S)-5-benzyl-N-(7,9-difluoro-2-oxo-2,3,4,5-tetrahydro-1H-benzo[b]azepin-3-yl)-4H-1,2,4-triazole-3-carboxamide; or
(S)-5-benzyl-N-(7-chloro-2-oxo-2,3,4,5-tetrahydro-1H-benzo[b]azepin-3-yl)-4H-1,2,4-triazole-3-carboxamide;
   or a tautomer thereof; or a salt thereof.

In one embodiment, there is provided a compound according to Formula (III):

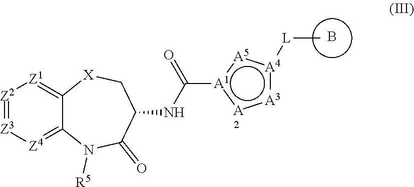

wherein:
X is CH₂ or NH;
Z¹ is CH;
Z² is CH or CR²;
Z³ is CH;
Z⁴ is CH or CR⁴;
R² and R⁴ are each independently selected from chloro or fluoro;
R⁵ is H or methyl;
L is CH₂;
A¹ and A⁴ are C, and A², A³, and A⁵ are each independently selected from N and NH (such that A¹-A²-A³-A⁴-A⁵-A¹ forms a triazolyl ring moiety),
B is a phenyl ring, optionally substituted by fluoro;
   or a salt, particularly a pharmaceutically acceptable salt, thereof;

provided that the compound is not:
(S)-5-benzyl-N-(2-oxo-2,3,4,5-tetrahydro-1H-benzo[b]azepin-3-yl)-4H-1,2,4-triazole-3-carboxamide;
(S)-5-benzyl-N-(1-methyl-2-oxo-2,3,4,5-tetrahydro-1H-benzo[b]azepin-3-yl)-4H-1,2,4-triazole-3-carboxamide;
(S)-5-benzyl-N-(7,9-difluoro-2-oxo-2,3,4,5-tetrahydro-1H-benzo[b]azepin-3-yl)-4H-1,2,4-triazole-3-carboxamide; or
(S)-5-benzyl-N-(7-chloro-2-oxo-2,3,4,5-tetrahydro-1H-benzo[b]azepin-3-yl)-4H-1,2,4-triazole-3-carboxamide;
   or a salt, particularly a pharmaceutically acceptable salt, thereof.

In one embodiment of the compounds of Formula (I), Formula (II), or Formula (III), X is CH₂. In another embodiment, X is NH.

In one embodiment of the compounds of Formula (I), (II) or (III), Z¹, Z², Z³, and Z⁴ are each CH. In another embodiment, Z¹, Z³, and Z⁴ are each CH and Z² is CR². In another embodiment, Z¹, Z², and Z³ are each CH and Z⁴ is CR⁴. In another embodiment, Z′ and Z³ are each CH, Z² is CR² and Z⁴ is CR⁴. In one embodiment of the compounds useful in this invention, R² is fluoro. In another embodiment, R² is chloro.

In one embodiment of the compounds useful in this invention, R⁴ is fluoro.

In one embodiment of the compounds useful in this invention, R⁵ is H.

In another embodiment, R⁵ is methyl.

In one embodiment of the compounds useful in this invention, B is unsubstituted phenyl.

In another embodiment, B is phenyl, substituted by a fluoro substituent.

In a specific embodiment, B is 2-fluorophenyl.

In one embodiment, X is NH, Z¹, Z², Z³, and Z⁴ are each CH, R⁵ is methyl, A¹ and A⁴ are C, A² and A⁵ are each independently selected from N and NH, L is CH₂ and B is a phenyl ring substituted by fluoro.

It will be appreciated that the present invention covers compounds of Formula (I), Formula (II), or Formula (III) as the free base, and as salts thereof, for example as a pharmaceutically acceptable salt thereof. In one embodiment, the invention relates to compounds of Formula (I), Formula (II), or Formula (III) in the form of a free base. In another embodiment, the invention relates to compounds of Formula (I), Formula (II), or Formula (III) or a pharmaceutically acceptable salt thereof. It will further be appreciated that compounds of Formula (I), Formula (II), or Formula (III) and salts thereof may exist in hydrated from, such as the monohydrate, dihydrate, or trihydrate.

Representative compounds useful in this invention include:
(S)—N-(9-fluoro-2-oxo-2,3,4,5-tetrahydro-1H-benzo[b][1,4]diazepin-3-yl)-5-(2-fluorobenzyl)-4H-1,2,4-triazole-3-carboxamide; or
(S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide;
   or a tautomer thereof;
   or a salt thereof, particularly a pharmaceutically acceptable salt thereof.

Representative compounds useful in this invention include a compound having the formula:

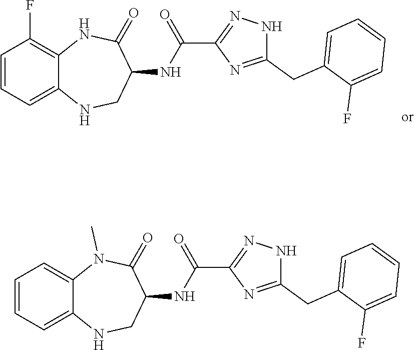 [SMILES: O=C(N[C@H]1CNc2cccc(F)c2NC1=O)c1n[nH]c(Cc2ccccc2F)n1 ; CN1C(=O)[C@@H](NC(=O)c2n[nH]c(Cc3ccccc3F)n2)CNc2ccccc21]

or a tautomer thereof;
or a salt thereof, particularly a pharmaceutically acceptable salt thereof.

In one embodiment, this invention is directed to (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide or a salt, particularly a pharmaceutically acceptable salt thereof. In one embodiment, the compound useful in this invention is (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide. In another embodiment, the compound useful in this invention is a salt of (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide. In another embodiment, the compound useful in this invention is a pharmaceutically acceptable salt of ((S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide. In another embodiment, the compound useful in this invention is ((S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide as the free base.

The compounds useful in this invention contain one asymmetric center (also referred to as a chiral center), a chiral carbon. The stereochemistry of the chiral carbon center present in compounds useful in this invention is generally represented in the compound names and/or in the chemical structures illustrated herein. Compounds useful in this invention containing a chiral center may be present as a racemic mixture, enantiomerically enriched mixture, or as an enantiomerically pure individual stereoisomer.

An individual stereoisomer of a compound useful in this invention may be resolved (or mixtures of stereoisomers may be enriched) using methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, a specific stereoisomer may be synthesized by asymmetric synthesis using optically active reagents, substrates, catalysts or solvents, or by converting one enantiomer to the other by asymmetric transformation.

The invention also includes various deuterated forms of the compounds of Formula (I), Formula (II), and Formula (III) Each available hydrogen atom attached to a carbon atom may be independently replaced with a deuterium atom. A person of ordinary skill in the art will know how to synthesize deuterated forms of the compounds of Formula (I), Formula (II), or Formula (III). For example, α-deuterated α-amino acids are commercially available or may be prepared by conventional techniques (see for example: Elemes, Y. and Ragnarsson, U. J. Chem. Soc., Perkin Trans. 1, 1996, 6, 537-40). Employing such compounds may allow for the preparation of compounds in which the hydrogen atom at a chiral center is replaced with a deuterium atom. Other commercially available deuterated starting materials may be employed in the preparation of deuterated analogs of the compounds useful in this invention (see for example: methyl-d₃-amine available from Aldrich Chemical Co., Milwaukee, Wis.), or they may be synthesized using conventional techniques employing deuterated reagents (e.g. by reduction using lithium aluminum deuteride or sodium borodeuteride or by metal-halogen exchange followed by quenching with D₂O or methanol-d₃).

The skilled artisan will appreciate that solvates (particularly, hydrates) of a compound of Formulas (I), (II), or (III), including solvates of salts of a compound of Formulas (I), (II), or (III), particularly a compound of any one of Formulas (I), (II), or (III), may be formed when solvent molecules are incorporated into the crystalline lattice during crystallization. The present invention includes within its scope all possible stoichiometric and non-stoichiometric salt and/or hydrate forms.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

It is to be understood that the references herein to a compound of Formulas (I), (II), or (III), or a salt thereof, includes a compound of Formulas (I), (II), or (III) as a free base or as a salt thereof, for example as a pharmaceutically acceptable salt thereof. Thus, in one embodiment, the invention is directed to a compound of Formulas (I), (II), or (III). In a further embodiment, the invention is directed to a pharmaceutically acceptable salt of a compound of Formulas (I), (II), or (III). In a further embodiment, the invention is directed to a compound of Formulas (I), (II), or (III), or a pharmaceutically acceptable salt thereof.

Because of their potential use in medicine, it will be appreciated that a salt of a compound of Formulas (I), (II), or (III) is preferably pharmaceutically acceptable. As used herein, the term “pharmaceutically acceptable” means a compound which is suitable for pharmaceutical use. Salts and solvates (e.g. hydrates and hydrates of salts) of the compounds of Formulas (I), (II), or (III) which are suitable for use in medicine are those wherein the counterion or associated solvent is pharmaceutically acceptable. Salts and solvates (e.g. hydrates and hydrates of salts) of the compounds useful in this invention which are suitable for use in medicine are those wherein the counterion or associated solvent is pharmaceutically acceptable. Salts and solvates having non-pharmaceutically acceptable counterions or associated solvents are within the scope of the present invention, for example, for use as intermediates in the preparation of other compounds useful in this invention and their salts and solvates.

Pharmaceutically acceptable salts include, amongst others, those described in Berge, J. Pharm. Sci., 66, 1-19, (1977) or those listed in P. H. Stahl and C. G. Wermuth, editors, Handbook of Pharmaceutical Salts; Properties, Selection and Use, Second Edition Stahl/Wermuth: Wiley-VCH/VHCA (2011) (see http://www.wiley.com/WileyCDA/WileyTitle/productCd-3906390519.html).

Suitable pharmaceutically acceptable salts can include acid addition salts.

Such acid addition salts can be formed by reaction of a compound of Formula (I), (II), or (III) (which, for example contains a basic amine or other basic functional group) with the appropriate acid, optionally in a suitable solvent such as an organic solvent, to give the salt which can be isolated by a variety of methods, including crystallization and filtration.

Salts may be prepared in situ during the final isolation and purification of a compound of Formula (I), (II), or (III). If a basic compound of Formula (I), (II), or (III) is isolated as a salt, the corresponding free base form of that compound may be prepared by any suitable method known to the art, including treatment of the salt with an inorganic or organic base.

This invention also provides for the conversion of one salt of a compound useful in this invention, e.g., a hydrochloride salt, into another salt of a compound useful in this invention, e.g., a sulfate salt. This invention also provides for the conversion of one pharmaceutically acceptable salt of a compound useful in this invention into another pharmaceutically acceptable salt of a compound useful in this invention.

It will be understood that if a compound of Formula (I), (II), or (III)) contains one or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of Formula (I), (II), or (III) are included within the scope of the invention, including sub-stoichiometric salts, for example where a counterion contains more than one acidic proton.

Certain compounds useful in this invention may form salts with one or more equivalents of an acid. The present invention includes within its scope all possible stoichiometric and non-stoichiometric salt forms.

It is to be further understood that the present invention includes within its scope all tautomeric forms of any free base form of the compounds useful in this invention as well as all possible stoichiometric and non-stoichiometric salt forms of all tautomeric forms of the compounds useful in this invention.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

For solvates of the compounds of Formulas (I), (II), or (III), including solvates of salts of the compounds of Formulas (I), (II), or (III), that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and EtOAc, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates, particularly hydrates. Accordingly, a compound useful in this invention includes a compound of Formula (I), (II), or (III), or a salt thereof, particularly a pharmaceutically acceptable salt thereof, or a hydrate thereof, a hydrate of a pharmaceutically acceptable salt of a compound of Formula (I), (II), or (III), and particularly includes each compound described in the Examples. Thus, the invention provides a compound of Formula (I), (II), or (III), or a salt thereof, especially a pharmaceutically acceptable salt thereof, as a solvate, particularly as a hydrate, such as a monohydrate, dihydrate, or trihydrate.

Because the compounds useful in this invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

A compound that inhibits RIP1 kinase, particularly a compound disclosed in WO2005/077344 (U.S. Pat. No. 7,491,743), WO2007/075772, WO2010/07556 (U.S. Pat. No. 9,586,880), WO2012/125544, WO2014/125444, WO2016/094846 (now U.S. Pat. No. 9,499,521), WO2016/101887, WO2016/185423, WO2017/004500 (now US 2017/0008877), U.S. Pat. No. 9,643,977, WO2017/096301, WO2017/069279, and/or U.S. Provisional Patent Application No. 62/424,047, filed Nov. 18, 2016, U.S. patent application Ser. No. 15/424,216, filed Feb. 3, 2017 (U.S. Pat. No. 9,815,850), U.S. patent application Ser. No. 15/200,058, filed Jul. 1, 2016, (the disclosures of each of which are incorporated by reference herein) or a compound of Formula (I), (II), or (III), or a pharmaceutically acceptable salt thereof, may be particularly useful for the treatment of RIP1 kinase-mediated diseases or disorders. These RIP1 kinase-mediated diseases or disorders are diseases or disorders that are mediated by activation of RIP1 kinase, and as such, are diseases or disorders where inhibition of RIP1 kinase would provide benefit. Such RIP1 kinase-mediated diseases or disorders are diseases/disorders which are likely to be regulated at least in part by programmed necrosis, apoptosis or the production of inflammatory cytokines, particularly: inflammatory bowel disease (including Crohn's disease and ulcerative colitis), psoriasis, retinal detachment, retinal degeneration, retinitis pigmentosa, macular degeneration, pancreatitis, atopic dermatitis, arthritis (including rheumatoid arthritis, spondyloarthritis, gout, juvenile idiopathic arthritis (systemic onset juvenile idiopathic arthritis (SoJIA)), psoriatic arthritis), systemic lupus erythematosus (SLE), Sjogren's syndrome, systemic scleroderma, anti-phospholipid syndrome (APS), vasculitis, osteoarthritis, liver damage/diseases (non-alcohol steatohepatitis, alcohol steatohepatitis, autoimmune hepatitis, autoimmune hepatobiliary diseases, primary sclerosing cholangitis (PSC), acetaminophen toxicity, hepatotoxicity), kidney damage/injury (nephritis, renal transplant, surgery, adjuvant therapy following solid tumor resection, administration of nephrotoxic drugs e.g. cisplatin, acute kidney injury (AKI)) Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), intracerebral hemorrhage, myocardial infarction (MI), atherosclerosis, Huntington's disease, Alzheimer's disease, Parkinson's disease, amyotrophic lateral sclerosis (ALS), neonatal brain injury, neonatal hypoxic brain injury, ischemic brain injury, traumatic brain injury, allergic diseases (including asthma and atopic dermatitis), peripheral nerve injury, burns, multiple sclerosis, type I diabetes, Wegener's granulomatosis, pulmonary sarcoidosis, Behcet's disease, interleukin-1 converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-kappa-B essential modulator gene (also known as IKK gamma or IKKG)), particularly, NEMO-deficiency syndrome, HOIL-1 deficiency (also known as RBCK1) heme-oxidized IRP2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, glaucoma, spinal cord injury, fibrosis, complement-mediated cytotoxicity, pancreatic cancer (particularly metastatic adenocarcinoma of the pancreas, pancreatic ductal adenocarcinoma and/or malignancies of the endocrine cells in the pancreas), hepatocellular carcinoma, mesothelioma, melanoma, colorectal cancer, acute myeloid leukemia, metastasis, glioblastoma, breast cancer, gallbladder cancer, clear cell renal carcinoma (cc-RCC), non-small cell lung carcinoma (NSCLC), acute liver failure, radiation protection/mitigation (radiation induced necrosis), auditory disorders such as noise-induced hearing loss and drugs associated with ototoxicity such as cisplatin, or for the treatment of cells ex vivo to preserve vitality and function.

In this invention, RIP1 kinase-mediated diseases or disorders are diseases or disorders that are mediated by activation of RIP1 kinase, and as such, are diseases or disorders where inhibition of RIP1 kinase would provide benefit. Such RIP1 kinase-mediated diseases or disorders are diseases/disorders which are likely to be regulated at least in part by programmed necrosis, apoptosis or the production of inflammatory cytokines, particularly inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, juvenile idiopathic arthritis (systemic onset juvenile idiopathic arthritis (SoJIA)), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH), autoimmune hepatitis, autoimmune hepatobiliary diseases, primary sclerosing cholangitis (PSC), acetaminophen toxicity, hepatotoxicity), non-alcohol steatohepatitis (NASH), alcohol steatohepatitis (ASH), autoimmune hepatitis, non-alcoholic fatty liver disease (NAFLD), kidney damage/injury (nephritis, renal transplant, surgery, administration of nephrotoxic drugs e.g. cisplatin, acute kidney injury (AKI)) Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, ischemic brain injury, traumatic brain injury allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-1 converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-kappa-B essential modulator gene (also known as IKK gamma or IKKG)), particularly, NEMO-deficiency syndrome, HOIL-1 deficiency (also known as RBCK1) heme-oxidized IRP2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, glaucoma, spinal cord injury, fibrosis, complement-mediated cytotoxicity, pancreatic ductal adenocarcinoma, hepatocellular carcinoma, mesothelioma, melanoma, metastasis, breast cancer, non-small cell lung carcinoma (NSCLC), radiation induced necrosis (acute radiation syndrome, radiation induced mucositis), ischemic kidney damage, ophthalmologic ischemia, intracerebral hemorrhage, subarachnoid hemorrhage, acute liver failure and radiation protection/mitigation, auditory disorders such as noise-induced hearing loss and drugs associated with ototoxicity such as cisplatin, or for the treatment of cells ex vivo to preserve vitality and function.

The treatment of the above-noted diseases/disorders may concern, more specifically, the amelioration of organ injury or damage sustained as a result of the noted diseases/disorders. For example, the compounds useful in this invention may be particularly useful for amelioration of brain tissue injury or damage following ischemic brain injury or traumatic brain injury, or for amelioration of heart tissue injury or damage following myocardial infarction, or for amelioration of brain tissue injury or damage associated with Huntington's disease, Alzheimer's disease or Parkinson's disease, or for amelioration of liver tissue injury or damage associated with non-alcohol steatohepatitis, alcohol steatohepatitis, autoimmune hepatitis autoimmune hepatobiliary diseases, or primary sclerosing cholangitis, or overdose of acetaminophen.

The compounds useful in this invention may be particularly useful for the amelioration of organ injury or damage sustained as a result of radiation therapy, or amelioration of spinal tissue injury or damage following spinal cord injury or amelioration of liver tissue injury or damage associated acute liver failure. The compounds useful in this invention may be particularly useful for amelioration of auditory disorders, such as noise-induced hearing loss or auditory disorders following the administration of ototoxic drugs or substances, e.g. cisplatin.

The compounds useful in this invention may be particularly useful for amelioration of solid organ tissue (particularly kidney, liver, and heart and/or lung) injury or damage following transplant or the administration of nephrotoxic drugs or substances e.g. cisplatin. It will be understood that amelioration of such tissue damage may be achieved where possible, by pre-treatment with a compound of Formula (I), (II), or (III), or a pharmaceutically acceptable salt thereof; for example, by pre-treatment of a patient prior to administration of cisplatin or pre-treatment of an organ or the organ recipient prior to transplant surgery. Amelioration of such tissue damage may be achieved by treatment with a compound of Formula (I), (II), or (III), or a pharmaceutically acceptable salt thereof, during transplant surgery. Amelioration of such tissue damage may also be achieved by short-term treatment of a patient with a compound of Formula (I), (II), or (III), or a pharmaceutically acceptable salt thereof, after transplant surgery.

Other RIP1 kinase-mediated diseases or disorders suitable for treatment using the compounds useful in this invention include: hemorrhagic shock, trauma (including multiple trauma), traumatic brain injury, burns (thermal injury), Stevens-Johnson Syndrome/toxic epidermal necrolysis, heat stroke, acute pancreatitis, critical illness (in general), chemotherapy, radiation injury, radiotherapy, sepsis, stroke, stroke-associated pneumonia, Systemic Inflammatory Response Syndrome (SIRS), Multi-Organ Dysfunction Syndrome (MODS), Acute Respiratory Distress Syndrome (ARDS), intestinal obstruction, liver cirrhosis, organ transplantation (for donors and recipients), major abdominal operations, abdominal aortic aneurysm repair, large bowel resections, ischemia-reperfusion injury (including organ (gut, brain, liver, kidney) ischemia, and limb ischemia), bowel ischemia (small intestine and large intestine), and cardiac surgery requiring cardio-pulmonary bypass. The compounds of Formulas (I), (II), or (III), or a pharmaceutically acceptable salt thereof, may be particularly useful for the prevention, delay of onset, amelioration, and/or treatment of diseases or disorders which result in RIP1-dependent inflammation of the gut epithelium, leading to bacterial translocation via blood or lymph to the systemic circulation. These diseases or disorders include hemorrhagic shock, trauma (including multiple trauma), traumatic brain injury, burns (thermal injury), heat stroke, acute pancreatitis, critical illness (in general), pneumonias, chemotherapy, radiation injury, radiotherapy, sepsis, septic shock, Stevens-Johnson syndrome, toxic epidermal necrolysis, stroke, stroke-associated pneumonia, Systemic Inflammatory Response Syndrome (SIRS), Multi-Organ Dysfunction Syndrome (MODS), Acute Respiratory Distress Syndrome (ARDS), intestinal obstruction, liver cirrhosis, organ transplantation (for donors and recipients), surgery, major abdominal operations, abdominal aortic aneurysm repair, large bowel resections, ischemia-reperfusion injury (including organ (gut, brain, liver, kidney) ischemia, and limb ischemia), bowel ischemia (small intestine and large intestine), and cardiac surgery requiring cardio-pulmonary bypass. It is anticipated that treatment of a patient suffering from one of such diseases or disorders (e.g., a burn injury) with a compound of Formulas (I), (II), or (III), or a pharmaceutically acceptable salt thereof, may prevent, delay the onset of, ameliorate or treat the resulting RIP1-dependent inflammation of the gut epithelium thereby preventing, delaying the onset of, or ameliorating the bacterial translocation via blood or lymph to the systemic circulation of the patient.

The compounds useful in this invention may be particularly useful for the treatment of inflammatory bowel disease (including Crohn's disease and ulcerative colitis), psoriasis, retinal detachment, retinitis pigmentosa, arthritis (including rheumatoid arthritis, spondyloarthritis, gout, osteoarthritis, and systemic onset juvenile idiopathic arthritis (SoJIA)), transplant rejection/organ transplantation, ischemia reperfusion injury of solid organs, sepsis, systemic inflammatory response syndrome, multiple sclerosis, and/or tumor necrosis factor receptor-associated periodic syndrome.

The compounds useful in this invention, particularly the compounds of Formulas (I) or Formulas (II) or (II), or a pharmaceutically acceptable salt thereof, may be particularly useful for the treatment of the following RIP1 kinase-mediated diseases or disorders.

In another embodiment, a compound that inhibits RIP1 kinase, particularly a compound disclosed in WO2005/077344 (U.S. Pat. No. 7,491,743), WO2007/075772, WO2010/07556 (U.S. Pat. No. 9,586,880), WO2012/125544, WO2014/125444, WO2016/094846 (now U.S. Pat. No. 9,499,521), WO2016/101887, WO2016/185423, WO2017/004500 (now US 2017/0008877), U.S. Pat. No. 9,643,977, WO2017/096301, WO2017/069279, and/or U.S. Provisional Patent Application No. 62/424,047, filed Nov. 18, 2016, U.S. Provisional Patent Application No. 62/585,267, filed Nov. 13, 2017, U.S. patent application Ser. No. 15/424,216, filed Feb. 3, 2017 (U.S. Pat. No. 9,815,850), U.S. patent application Ser. No. 15/200,058, filed Jul. 1, 2016, (the disclosures of each of which are incorporated by reference herein) may be particularly useful for the treatment of the following RIP1 kinase-mediated diseases or disorders.

In one embodiment of this invention, the RIP1 kinase-mediated disease or disorder is a solid tumor.

In another embodiment, this invention is directed to a method of treating a RIP1 kinase-mediated disease or disorder comprising administering a therapeutically effective amount of a compound that inhibits RIP1 kinase to a human in need thereof.

In yet another embodiment, this invention is directed to a method of treating a RIP1 kinase-mediated disease or disorder comprising administering a therapeutically effective amount of a compound that inhibits RIP1 kinase in combination with an immuno-modulator a human in need thereof.

In one embodiment, the human has a solid tumor.

Accordingly, in one embodiment, this invention is directed to a method of treating a RIP1 kinase-mediated disease or disorder comprising administering a therapeutically effective amount of a compound that inhibits RIP1 kinase to a human in need thereof, wherein the compound that inhibits RIP1 kinase is a compound of Formulas (I) (a compound of WO2014/125444) and Formula (II), or a pharmaceutically acceptable salt thereof, or is a compound disclosed in WO2005/077344 (U.S. Pat. No. 7,491,743), WO2007/075772, WO2010/07556 (U.S. Pat. No. 9,586,880), WO2012/125544, WO2014/125444, WO2016/094846 (now U.S. Pat. No. 9,499,521), WO2016/101887, WO2016/185423, WO2017/004500 (now US 2017/0008877), U.S. Pat. No. 9,643,977, WO2017/096301, WO2017/069279, and/or U.S. Provisional Patent Application No. 62/424,047, filed Nov. 18, 2016, U.S. Provisional Patent Application No. 62/585,267, filed Nov. 13, 2017, U.S. patent application Ser. No. 15/424,216, filed Feb. 3, 2017 (U.S. Pat. No. 9,815,850), U.S. patent application Ser. No. 15/200,058, filed Jul. 1, 2016, (the disclosures of each of which are incorporated by reference herein), and wherein the human has a solid tumor.

In another embodiment, this invention is directed to a method of treating a RIP1 kinase-mediated cancer comprising administering a therapeutically effective amount of a compound that inhibits RIP1 kinase in combination with at least one other therapeutically active agent, specifically, an immuno-modulator, to a human in need thereof, wherein the compound that inhibits RIP1 kinase is a compound of Formulas (I) and (II), or a pharmaceutically acceptable salt thereof, or is a compound disclosed in WO2005/077344 (U.S. Pat. No. 7,491,743), WO2007/075772, WO2010/07556 (U.S. Pat. No. 9,586,880), WO2012/125544, WO2014/125444, WO2016/094846 (now U.S. Pat. No. 9,499,521), WO2016/101887, WO2016/185423, WO2017/004500 (now US 2017/0008877), U.S. Pat. No. 9,643,977, WO2017/096301, WO2017/069279, and/or U.S. Provisional Patent Application No. 62/424,047, filed Nov. 18, 2016, U.S. Provisional Patent Application No. 62/585,267, filed Nov. 13, 2017, U.S. patent application Ser. No. 15/424,216, filed Feb. 3, 2017 (U.S. Pat. No. 9,815,850), U.S. patent application Ser. No. 15/200,058, filed Jul. 1, 2016, (the disclosures of each of which are incorporated by reference herein), and wherein the human has a solid tumor.

In one aspect, the tumor is selected from head and neck cancer, gastric cancer, melanoma, renal cell carcinoma (RCC), esophageal cancer, non-small cell lung carcinoma (NSCLC), prostate cancer, colorectal cancer, ovarian cancer, pancreatic cancer, and pancreatic ductal adenocarcinoma. In one aspect, the human has one or more of the following: colorectal cancer (CRC), esophageal cancer, cervical, bladder, breast cancer, head and neck cancer, ovarian cancer, melanoma, renal cell carcinoma (RCC), EC squamous cell carcinoma, non-small cell lung carcinoma, mesothelioma, prostate cancer, and pancreatic ductal adenocarcinoma. In another aspect, the human has a liquid tumor such as diffuse large B cell lymphoma (DLBCL), multiple myeloma, chronic lyl) homblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, astrocytomas, Bannayan-Zonana syndrome, Cowden disease, Lhermitte-Duclos disease, breast cancer, triple negative breast cancer, inflammatory breast cancer, Wilm's tumor, Ewing's sarcoma, Rhabdomyosarcoma, ependymoma, medulloblastoma, colon cancer, head and neck cancer (including squamous cell carcinoma of head and neck), kidney cancer, lung cancer (including lung squamous cell carcinoma, lung adenocarcinoma, lung small cell carcinoma, and non-small cell lung carcinoma), liver cancer (including hepatocellular carcinoma), melanoma, ovarian cancer, pancreatic cancer (including squamous pancreatic cancer), prostate cancer, sarcoma, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkin's lymphoma, Non-Hodgkin's lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, cancer of the uterus, renal cancer (including kidney clear cell cancer, kidney papillary cancer, renal cell carcinoma), mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

Accordingly, one embodiment of this invention is directed to a method of inhibiting RIP1 kinase comprising contacting said kinase with a compound useful in this invention. In another embodiment, this invention is directed to a method of inhibiting RIP1 kinase comprising contacting a cell with a compound useful in this invention.

Another embodiment of this invention is directed to a method of treating a RIP1 kinase-mediated disease or disorder (specifically, a disease or disorder recited herein) comprising administering a therapeutically effective amount of a compound that inhibits RIP1 kinase to a human in need thereof.

Another embodiment of this invention is directed to a method of treating a RIP1 kinase-mediated disease or disorder (specifically, a disease or disorder recited herein) comprising administering a therapeutically effective amount of a compound that inhibits RIP1 kinase with at least one other therapeutically active agent to a human in need thereof.

In another embodiment, the invention is directed to a method of treating a RIP1 kinase-mediated disease or disorder comprising administering a therapeutically effective amount of a compound useful in this invention, particularly a compound of Formula (I), (II), or (III), or a salt, particularly a pharmaceutically acceptable salt thereof, to a human in need thereof. In another embodiment, the invention is directed to a method of treating a RIP1 kinase-mediated disease or disorder comprising administering a therapeutically effective amount of a compound useful in this invention, particularly a compound of Formula (I), (II), or (III), or a salt, particularly a pharmaceutically acceptable salt thereof, with at least one other therapeutically active agent to a human in need thereof.

Specifically, this invention provides a method of treating a RIP1 kinase-mediated disease or disorder (specifically, a disease or disorder recited herein) comprising administering a therapeutically effective amount of a compound described herein, or a pharmaceutically acceptable salt thereof, to a human in need thereof. More specifically, this invention provides a method of treating a RIP1 kinase-mediated disease or disorder (specifically, a disease or disorder recited herein) comprising administering a therapeutically effective amount of a compound described herein, or a pharmaceutically acceptable salt thereof, with at least one other therapeutically active agent, to a human in need thereof.

In one specific embodiment, the invention is directed to a method of treating a RIP1 kinase-mediated disease or disorder (specifically, a disease or disorder recited herein) comprising administering a therapeutically effective amount of (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, or a pharmaceutically acceptable salt thereof, to a human in need thereof.

In another embodiment, this invention provides a compound that inhibits RIP1 kinase for use in therapy. This invention also provides a compound useful in this invention, particularly a compound of Formula (I), (II), or (III), or a pharmaceutically acceptable salt thereof, for use in therapy. Specifically, this invention provides a compound described herein, or a pharmaceutically acceptable salt thereof, for use in therapy. More specifically, this invention provides (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, or a pharmaceutically acceptable salt thereof, for use in therapy. More specifically, this invention provides (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, for use in therapy.

In another embodiment, this invention provides a compound that inhibits RIP1 kinase for use in the treatment of a RIP1 kinase-mediated disease or disorder (for example, a disease or disorder recited herein). In another embodiment, this invention provides a compound that inhibits RIP1 kinase with at least one other therapeutically active agent for use in the treatment of a RIP1 kinase-mediated disease or disorder (for example, a disease or disorder recited herein).

This invention particularly provides a compound that inhibits RIP1 kinase, particularly a compound of Formula (I), (II), or (III), or a pharmaceutically acceptable salt thereof, for use in the treatment of a RIP1 kinase-mediated disease or disorder.

This invention particularly provides a compound that inhibits RIP1 kinase, particularly a compound of Formula (I), (II), or (III), or a pharmaceutically acceptable salt thereof, with at least one other therapeutically active agent, for use in the treatment of a RIP1 kinase-mediated disease or disorder.

Specifically, this invention provides a compound described herein, or a pharmaceutically acceptable salt thereof, for use in the treatment of a RIP1 kinase-mediated disease or disorder. More specifically, this invention provides (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, or a pharmaceutically acceptable salt thereof, for use in the treatment of a RIP1 kinase-mediated disease or disorder (for example, a disease or disorder recited herein). This invention further provides (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, for use in the treatment of a RIP1 kinase-mediated disease or disorder (for example, a disease or disorder recited herein).

This invention specifically provides for the use of a compound that inhibits RIP1 kinase as an active therapeutic substance. This invention specifically provides for the use of a compound of Formula (I), (II), or (III), or a pharmaceutically acceptable salt thereof, as an active therapeutic substance. More specifically, this invention provides for the use of a compound described herein for the treatment of a RIP1 kinase-mediated disease or disorder. Accordingly, the invention provides for the use of a compound of Formula (I), (II), or (III), or a pharmaceutically acceptable salt thereof, as an active therapeutic substance in the treatment of a human in need thereof with a RIP1 kinase-mediated disease or disorder. Specifically, this invention provides the invention provides for the use of (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, or a pharmaceutically acceptable salt thereof, as an active therapeutic substance in the treatment of a human in need thereof with a RIP1 kinase-mediated disease or disorder. More specifically, this invention provides the invention provides for the use of (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, as an active therapeutic substance in the treatment of a human in need thereof with a RIP1 kinase-mediated disease or disorder.

The invention further provides for the use of a compound that inhibits RIP1 kinase in the manufacture of a medicament for the treatment of a RIP1 kinase-mediated disease or disorder, for example the diseases and disorders recited herein. The invention further provides for the use of a compound of Formula (I), (II), or (III), or a pharmaceutically acceptable salt thereof, in the manufacture of a medicament for the treatment of a RIP1 kinase-mediated disease or disorder. Specifically, the invention provides for the use of a compound described herein, or a pharmaceutically acceptable salt thereof, in the manufacture of a medicament for the treatment of a RIP1 kinase-mediated disease or disorder. More specifically, the invention provides for the use of (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, or a pharmaceutically acceptable salt thereof, in the manufacture of a medicament for the treatment of a RIP1 kinase-mediated disease or disorder, for example the diseases and disorders recited herein. Specifically, the invention provides for the use of (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, in the manufacture of a medicament for the treatment of a RIP1 kinase-mediated disease or disorder, for example the diseases and disorders recited herein.

RIP1 kinase-mediated disease or disorders specifically suitable for treatment using a compound that inhibits RIP1 kinase are diseases and disorders selected from inflammatory bowel disease (including Crohn's disease and ulcerative colitis), psoriasis, retinal detachment, retinitis pigmentosa, arthritis (including rheumatoid arthritis, spondyloarthritis, gout, osteoarthritis, and systemic onset juvenile idiopathic arthritis (SoJIA)), transplant rejection, organ transplantation (for donors and recipients), multiple sclerosis, tumor necrosis factor receptor-associated periodic syndrome, multiple organ dysfunction syndrome (MODS), thermal injury/burn, systemic inflammatory response syndrome (SIRS), radiation injury, radiotherapy, chemotherapy, pneumonias, hemorrhagic shock, trauma (including multiple trauma), traumatic brain injury, acute pancreatitis, critical illness (in general), sepsis, septic shock, Stevens-Johnson syndrome, toxic epidermal necrolysis, stroke, heat stroke, stroke-associated pneumonia, Multi-Organ Dysfunction Syndrome (MODS), Acute Respiratory Distress Syndrome (ARDS), intestinal obstruction, liver cirrhosis, surgery, major abdominal operations, abdominal aortic aneurysm repair, large bowel resections, ischemia reperfusion injury (including ischemia reperfusion injury of solid organs, (gut, brain, liver, kidney), and limb ischemia), bowel ischemia (small intestine and large intestine), and cardiac surgery requiring cardio-pulmonary bypass.

Other RIP1 kinase-mediated disease or disorders specifically suitable for treatment using a compound that inhibits RIP1 kinase, wherein the compound that inhibits RIP1 kinase is a compound disclosed in WO2005/077344 (U.S. Pat. No. 7,491,743), WO2007/075772, WO2010/07556 (U.S. Pat. No. 9,586,880), WO2012/125544, WO2014/125444, WO2016/094846 (now U.S. Pat. No. 9,499,521), WO2016/101887, WO2016/185423, WO2017/004500 (now US 2017/0008877), U.S. Pat. No. 9,643,977, WO2017/096301, WO2017/069279, and/or U.S. Provisional Patent Application No. 62/424,047, filed Nov. 18, 2016, U.S. patent application Ser. No. 15/424,216, filed Feb. 3, 2017 (U.S. Pat. No. 9,815,850), U.S. patent application Ser. No. 15/200,058, filed Jul. 1, 2016, (the disclosures of each of which are incorporated by reference herein, or is a compound of Formula (I), (II), or (III), or a pharmaceutically acceptable salt thereof, are diseases and disorders selected from pancreatic cancer, metastatic adenocarcinoma of the pancreas, pancreatic ductal adenocarcinoma, a malignancy of the endocrine cells in the pancreas, hepatocellular carcinoma, mesothelioma, melanoma, colorectal cancer, acute myeloid leukemia, metastasis, glioblastoma, breast cancer, gallbladder cancer, clear cell renal carcinoma, non-small cell lung carcinoma, and radiation induced necrosis.

Accordingly, in one embodiment, a compound that inhibits RIP1 kinase is (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide or (S)-5-benzyl-N-(7,9-difluoro-2-oxo-2,3,4,5-tetrahydro-1H-benzo[b]azepin-3-yl)-4H-1,2,4-triazole-3-carboxamide; or a tautomer thereof; or a pharmaceutically acceptable salt thereof.

In one embodiment, a compound that inhibits RIP1 kinase is (S)-5-benzyl-N-(5-methyl-4-oxo-2,3,4,5-tetrahydrobenzo[b][1,4]oxazepin-3-yl)-4H-1,2,4-triazole-3-carboxamide; or a tautomer thereof; or a pharmaceutically acceptable salt thereof. In another embodiment, a compound that inhibits RIP1 kinase is (S)-5-benzyl-N-(5-methyl-4-oxo-2,3,4,5-tetrahydrobenzo[b][1,4]oxazepin-3-yl)-4H-1,2,4-triazole-3-carboxamide; or a tautomer thereof.

In another embodiment, a compound that inhibits RIP1 kinase is:

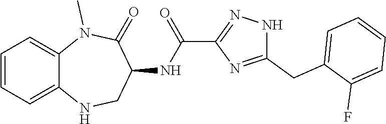 [SMILES: CN1C(=O)[C@@H](NC(=O)c2n[nH]c(Cc3ccccc3F)n2)CNc2ccccc21]

or a pharmaceutically acceptable salt thereof, or a tautomer thereof.

In another embodiment, a compound that inhibits RIP1 kinase is:

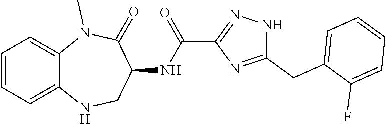 [SMILES: CN1C(=O)[C@@H](NC(=O)c2n[nH]c(Cc3ccccc3F)n2)CNc2ccccc21]

or a tautomer thereof.

In another embodiment, a compound that inhibits RIP1 kinase is:

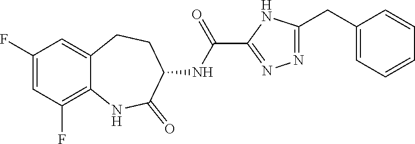 [SMILES: O=C(N[C@H]1CCc2cc(F)cc(F)c2NC1=O)c1nnc(Cc2ccccc2)[nH]1]

or a pharmaceutically acceptable salt thereof, or a tautomer thereof.

In another embodiment, a compound that inhibits RIP1 kinase is:

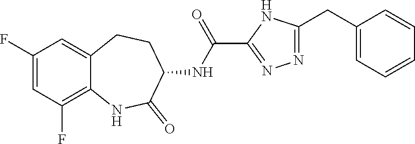 [SMILES: O=C(N[C@H]1CCc2cc(F)cc(F)c2NC1=O)c1nnc(Cc2ccccc2)[nH]1]

or a tautomer thereof.

In another embodiment, a compound that inhibits RIP1 kinase is:

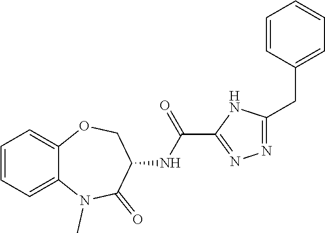 [SMILES: CN1C(=O)[C@@H](NC(=O)c2nnc(Cc3ccccc3)[nH]2)COc2ccccc21]

or a pharmaceutically acceptable salt thereof, or a tautomer thereof.

In another embodiment, a compound that inhibits RIP1 kinase is:

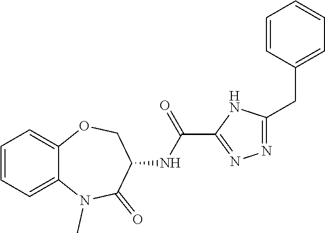 [SMILES: CN1C(=O)[C@@H](NC(=O)c2nnc(Cc3ccccc3)[nH]2)COc2ccccc21]

or a tautomer thereof.

In one embodiment, a compound disclosed in U.S. Pat. No. 9,815,850 (U.S. patent application Ser. No. 15/424,216, the disclosure of which is incorporated by reference herein) that inhibits RIP1 kinase is a compound having the formula:

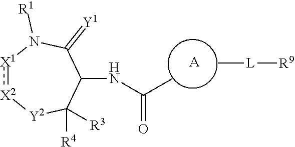

or a pharmaceutically acceptable salt, tautomer, stereoisomer or mixture of stereoisomers thereof, wherein:
R′ is H or optionally substituted C₁-C₆ alkyl;
X¹ and X² together form an optionally substituted pyridyl:

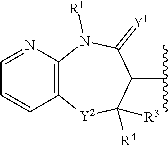

Y¹ is O;
Y² is —O—;
R³ and R⁴ are independently H, halo, or optionally substituted C₁-C₆ alkyl, or R³ and R⁴ together with the carbon atom to which they are attached, form an optionally substituted cycloalkyl or optionally substituted heterocyclyl ring;
A is an optionally substituted cycloalkyl, optionally substituted heterocyclyl ring or optionally substituted heteroaryl ring;
L is absent, —O—, —S—, —S(O)—, —S(O)₂—; —NR⁷— or C(R⁸)₂—;
R is H or optionally substituted C₁-C₆ alkyl;
each R⁸ is independently H, halo, or optionally substituted C₁-C₆ alkyl, or two R⁸ together with the carbon atom to which they are attached, form an optionally substituted cycloalkyl or optionally substituted heterocyclyl ring; and
R⁹ is optionally substituted cycloalkyl, optionally substituted heterocyclyl, optionally substituted aryl or optionally substituted heteroaryl;
wherein each optionally substituted pyridyl, optionally substituted C₁-C₆ alkyl, optionally substituted cycloalkyl, optionally substituted heterocyclyl, optionally substituted aryl, and optionally substituted heteroaryl ring is independently optionally substituted by one or more substituents, provided that the designated atom's normal valence is not exceeded, selected from alkyl, alkenyl, alkynyl, alkoxy, alkylthio, acyl, amido, amino, amidino, aryl, aralkyl, azido, carbamoyl, cyano, cycloalkyl, cycloalkylalkyl, guanadino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)₂OH, —S(O)₂OH, sulfonamido, —SH, thioxo, N-oxide, Si(R¹⁰⁰)₃ wherein each R¹⁰⁰ is independently hydrogen, alkyl, alkenyl, alkynyl, heteroalkyl, cycloalkyl, aryl, heteroaryl, or heterocyclyl, —OC(O)R, and —C(O)OR, wherein R is hydrogen, alkyl, alkenyl, alkynyl, cycloalkyl, heterocyclyl, aryl, heteroalkyl, or heteroaryl; and
further wherein:
each cycloalkyl is independently a saturated or partially unsaturated cyclic alkyl group of from 3 to 20 ring carbon atoms having a single ring or multiple rings,
wherein the cycloalkyl may be fused, bridged, or spiro;
each heterocyclyl is independently a saturated or unsaturated cyclic alkyl group of from 2 to 20 ring carbon atoms with one to five ring heteroatoms independently selected from nitrogen, oxygen and sulfur, and may comprise one or more oxo (C═O) or N-oxide (N—O—) moieties and/or a single ring or multiple rings wherein the multiple rings may be fused, bridged, or spiro; and
each heteroaryl is independently an aromatic group having 1 to 20 ring carbon atoms, a single ring, multiple rings, or multiple fused rings, with one to five ring heteroatoms independently selected from nitrogen, oxygen, and sulfur.

In one embodiment, a compound that inhibits RIP1 kinase is a compound having the formula:

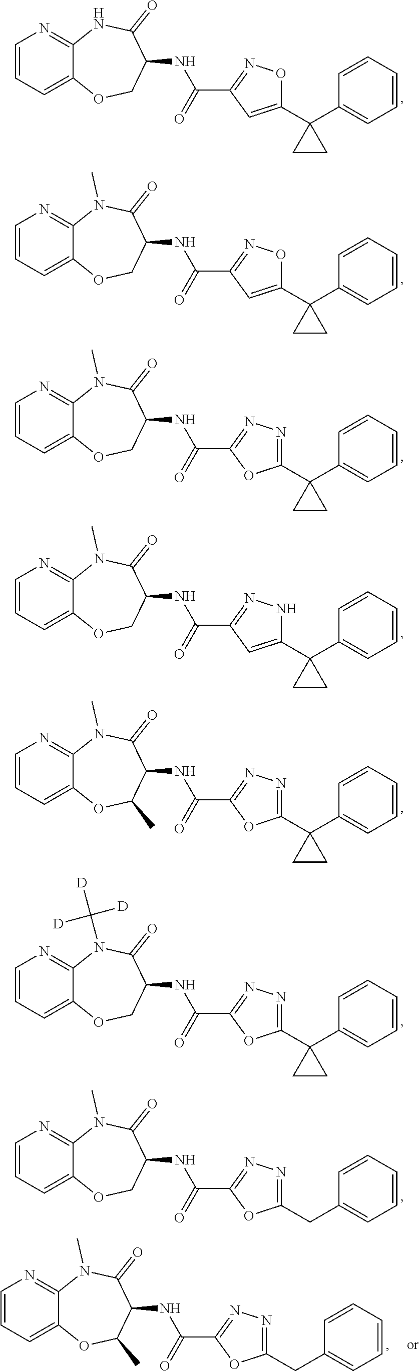

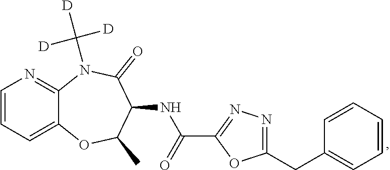 [SMILES: [2H]C([2H])([2H])N1C(=O)[C@@H](NC(=O)c2nnc(Cc3ccccc3)o2)[C@@H](C)Oc2cccnc21]

or a pharmaceutically acceptable salt thereof.

In one embodiment, a compound disclosed in U.S. Pat. No. 9,499,521 (the disclosure of which is incorporated by reference herein, corresponding to WO2016/094846) that inhibits RIP1 kinase is a compound having the formula:

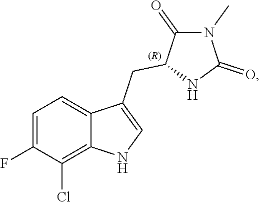

or a pharmaceutically acceptable salt thereof.

In one embodiment, a compound disclosed in WO2017/004500 (now US 2017/0008877, the disclosure of which is incorporated by reference herein) that inhibits RIP1 kinase is a compound having the formula:

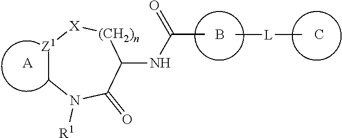

or a pharmaceutically acceptable salt thereof, wherein
R¹ is selected from the group consisting of H and unsubstituted C₁-C₄ alkyl;
the A ring is selected from the group consisting of cyclopropyl, 6 membered aryl, and 5 to 6 membered heteroaryl having 1 to 3 heteroatoms selected from the group consisting of nitrogen, oxygen and sulfur; wherein the A ring is optionally substituted with:
   (a) 1 to 3 substituents selected from the group consisting of halogen, C₁-C₆ alkyl, C₁-C₆ haloalkyl, C₃-C₆ cycloalkyl, C₁-C₆ alkoxy, C₁-C₆ haloalkoxy, C₁-C₆ thioalkyl, cyano, phenyl, benzyl, CH₂—(C₃-C₆ cycloalkyl), and CH₂CH₂—(C₃-C₆ cycloalkyl); wherein if a nitrogen atom in the A ring is substituted, the substituent is not halogen, C₁-C₆ alkoxy, C₁-C₆ haloalkoxy, C₁-C₆ thioalkyl, or cyano;
   (b) 1 substituent selected from the group consisting of C₄-C₆ heterocyclyl, C₅-C₆ heteroaryl, CH₂—(C₄-C₆ heterocyclyl), CH₂CH₂—(C₄-C₆ heterocyclyl), CH₂—(C₅-C₆ heteroaryl), CH₂CH₂—(C₅-C₆ heteroaryl); and optionally a second substituent selected from the group consisting of C₁-C₆ alkyl, C₁-C₆ haloalkyl, C₁-C₆ alkoxy, and C₁-C₆ haloalkoxy; or
   (c) two adjacent substituents which together form phenyl, C₅-C₆ heteroaryl, C₄-C₆ heterocyclyl or C₄-C₆ cycloalkyl;
the B ring is tetrazolyl or a 5 to 6 membered heteroaryl having 1 to 3 heteroatoms selected from the group consisting of nitrogen, oxygen and sulfur; wherein the B ring is optionally substituted with 1 to 2 substituents selected from the group consisting of halogen, C₁-C₄ alkyl, C₃-C₄ cycloalkyl, C₁-C₄ haloalkyl, C₁-C₄ alkoxy, C₁-C₄ haloalkoxy and cyano; and wherein if a nitrogen atom in the B ring is substituted, the substituent is not halogen, C₁-C₄ alkoxy, C₁-C₄ haloalkoxy, C₁-C₄ thioalkyl, or cyano;
the C ring is selected from the group consisting of phenyl, 5 to 6 membered heteroaryl, 5 to 7 membered cycloalkyl, and 5 to 7 membered heterocyclyl; wherein the C ring is optionally substituted with:
   (a) 1 to 4 substituents selected from the group consisting of halogen, C₁-C₆ alkyl, C₁-C₆ haloalkyl, C₃-C₆ cycloalkyl, C₁-C₆ alkoxy, C₁-C₆ haloalkoxy, C₁-C₆thioalkyl, cyano, phenyl, benzyl, CH₂—(C₃-C₆ cycloalkyl), and CH₂CH₂—(C₃-C₆ cycloalkyl); wherein if a nitrogen atom in the C ring is substituted, the substituent is not halogen, C₁-C₆ alkoxy, C₁-C₆ haloalkoxy, C₁-C₆ thioalkyl, or cyano;
   (b) 1 to 2 substituents selected from the group consisting of C₁-C₆ alkyl, C₁-C₆ haloalkyl, C₁-C₆ alkoxy, C₁-C₆ haloalkoxy, CH₂(C₄-C₆ heterocyclyl), CH₂CH₂—(C₄-C₆ heterocyclyl), and unsubstituted C₅-C₆ heteroaryl; or
   (c) two adjacent substituents which together form phenyl, C₅-C₆ heteroaryl, C₄-C₆ heterocyclyl or C₄-C₆ cycloalkyl;
L is selected from the group consisting of a bond, O, S, NH, NCH₃, (CH₂)ₘ, CH(CH₃), C(CH₃)₂, CF₂, CH₂O, CH₂S, CH(OH), CH₂NH, and CH₂N(CH₃), or L is absent such that the B ring and the C ring are fused;
X is selected from the group consisting of CH₂, C(CH₃)₂, CF₂ and CHCF3;
Z¹ is N.
m is 1 or 4; and
n is 1;
provided that if the A ring is 6 membered aryl or 6 membered heteroaryl, L is absent such that the B ring and the C ring are fused;
further provided that if the A ring is a 5 to 6 membered heteroaryl having 3 heteroatoms, two of said heteroatoms must be nitrogen;
further provided that if the A ring is unsubstituted 6 membered aryl and Lis absent, the fused B, and C rings are not unsubstituted indolyl or indolyl substituted by one or two halogen atoms; and
further provided that if the B ring is tetrazolyl, L is selected from the group consisting of CH₂, CH(CH₃) CH(CH₃)₂, C(CH₃)₂, CF₂; and the C ring is phenyl.

In another embodiment, a compound disclosed in WO2017/004500 (now US 2017/0008877, the disclosure of which is incorporated by reference herein) that inhibits RIP1 kinase is:
(S)-1-benzyl-N-(4-methyl-5-oxo-5,6,7,8-tetrahydro-4H-pyrazolo[1,5-a][1,3]diazepin-6-yl)-1H-1,2,4-triazole-3-carboxamide;
(S)-1-benzyl-N-(4-methyl-5-oxo-2-(trifluoromethyl)-5,6,7,8-tetrahydro-4H-pyrazolo[1,5-a][1,3]diazepin-6-yl)-1H-1,2,4-triazole-3-carboxamide
(S)—N—((S)-4-methyl-5-oxo-5,6,7,8-tetrahydro-4H-pyrazolo[1,5-a][1,3]diazepin-6-yl)-5-phenyl-6,7-dihydro-5H-pyrrolo[1,2-b][1,2,4]triazole-2-carboxamide;
(S)-1-benzyl-N-(2-cyclopropyl-4-methyl-5-oxo-5,6,7,8-tetrahydro-4H-pyrazolo[1,5-a][1,3]diazepin-6-yl)-1H-1,2,4-triazole-3-carboxamide;
(S)-1-benzyl-N-(2,4-dimethyl-5-oxo-5,6,7,8-tetrahydro-4H-pyrazolo[1,5-a] [1,3]diazepin-6-yl)-1H-1,2,4-triazole-3-carboxamide;
(S)-1-(2,6-difluorobenzyl)-N-(2,4-dimethyl-5-oxo-5,6,7,8-tetrahydro-4H-pyrazolo[1,5-a][1,3]diazepin-6-yl)-1H-1,2,4-triazole-3-carboxamide;
(S)—N-(2-cyclopropyl-4-methyl-5-oxo-5,6,7,8-tetrahydro-4H-pyrazolo[1,5-a][1,3]diazepin-6-yl)-1-(2,6-difluorobenzyl)-1H-1,2,4-triazole-3-carboxamide;
(S)—N-(2-cyclopropyl-4-methyl-5-oxo-5,6,7,8-tetrahydro-4H-pyrazolo[1,5-a][1,3]diazepin-6-yl)-1-(3,5-difluorobenzyl)-1H-1,2,4-triazole-3-carboxamide;
(S)—N-(2-cyclopropyl-4-methyl-5-oxo-5,6,7,8-tetrahydro-4H-pyrazolo[1,5-a][1,3]diazepin-6-yl)-1-(2,5-difluorobenzyl)-1H-1,2,4-triazole-3-carboxamide;
(S)-1-(2,5-difluorobenzyl)-N-(2,4-dimethyl-5-oxo-5,6,7,8-tetrahydro-4H-pyrazolo[1,5-a][1,3]diazepin-6-yl)-1H-1,2,4-triazole-3-carboxamide;
(S)—N-(2-cyclopropyl-4-methyl-5-oxo-5,6,7,8-tetrahydro-4H-pyrazolo[1,5-a][1,3]diazepin-6-yl)-1-(2,3-dichlorobenzyl)-1H-1,2,4-triazole-3-carboxamide;
(S)—N-(2-cyclopropyl-4-methyl-5-oxo-5,6,7,8-tetrahydro-4H-pyrazolo[1,5-a][1,3]diazepin-6-yl)-1-(2,4-dichlorobenzyl)-1H-1,2,4-triazole-3-carboxamide;
(S)-1-benzyl-N-(2-isopropyl-4-methyl-5-oxo-5,6,7,8-tetrahydro-4H-pyrazolo[1,5-a][1,3]diazepin-6-yl)-1H-1,2,4-triazole-3-carboxamide;
(S)—N-(2-ethyl-4-methyl-5-oxo-5,6,7,8-tetrahydro-4H-pyrazolo[1,5-a][1,3]diazepin-6-yl)-1-(2-fluorobenzyl)-1H-1,2,4-triazole-3-carboxamide;
(R)-5-(2-fluorophenyl)-N—((S)-4-methyl-5-oxo-5,6,7,8-tetrahydro-4H-pyrazolo[1,5-a][1,3]diazepin-6-yl)-6,7-dihydro-5H-pyrrolo[1,2-b] [1,2,4]triazole-2-carboxamide;
(5R)-5-phenyl-N-[(6S)-2,4-dimethyl-5-oxo-7,8-dihydro-6H-pyrazolo[1,5-a][1,3]diazepin-6-yl]-6,7-dihydro-5H-pyrrolo[1,2-b][1,2,4]triazole-2-carboxamide; or
(5R)-5-(2-fluorophenyl)-N-[(6S)-4-methyl-5-oxo-7,8-dihydro-6H-pyrazolo[1,5-a][1,3]diazepin-6-yl]-6,7-dihydro-5H-pyrrolo[1,2-b][1,2,4]triazole-2-carboxamide;
   or a pharmaceutically acceptable salt thereof.

In one embodiment, a compound disclosed in WO2016/185423 (the disclosure of which is incorporated by reference herein) that inhibits RIP1 kinase is a compound having the following formula:

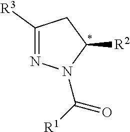

wherein:
R¹ is (C₁-C₄)alkoxy-CH₂—, phenyl(C₁-C₄)alkoxy-CH₂—, or a substituted or unsubstituted (C₂-C₆)alkyl, (C₂-C₄)alkynyl, (C₃-C₆)cycloalkyl, (C₃-C₆)cycloalkyl-(C₁-C₄)alkyl- group, or a substituted or unsubstituted 5-6 membered heterocycloalkyl group further optionally substituted by halogen or (C₁-C₄)alkyl,
   wherein said substituted (C₂-C₆)alkyl, (C₃-C₆)cycloalkyl, (C₃-C₆)cycloalkyl-alkyl-, or 5-6 membered heterocycloalkyl group is substituted by 1, 2 or 3 substituents independently selected from hydroxyl, (benzyloxy)carbonyl)amino, cyano, halogen, (C₁-C₄)alkyl, halo(C₁-C₄)alkyl, (C₁-C₄)alkoxy, (C₁-C₄)alkyl-CO—, cyano(C₁-C₄)alkyl-CO—, (C₁-C₄)alkoxy-(C₁-C₄)alkyl-CO—, (C₁-C₄)alkoxy-CO—, (C₁-C₄)alkylNHCO—, ((C₁-C₄)alkyl)((C₁-C₄)alkyl)NCO—, halo(C₁-C₄)alkyl-CO—, optionally substituted (C₃-C₆)cycloalkyl-CO—, optionally substituted (C₃-C₆)cycloalkyl-(C₁-C₄)alkyl-CO—, optionally substituted phenyl-CO—, optionally substituted phenyl-SO₂—, optionally substituted phenyl(C₁-C₄)alkyl-CO—, optionally substituted 5-6 membered heteroaryl-CO—, and optionally substituted 9-10 membered heteroaryl-CO—,
      wherein said optionally substituted (C₃-C₆)cycloalkyl-CO—, optionally substituted (C₃-C₆)cycloalkyl-(C₁-C₄)alkyl-CO—, optionally substituted phenyl-CO—, optionally substituted phenyl-SO₂—, optionally substituted phenyl(C₁-C₄)alkyl-CO—, optionally substituted 5-6 membered heteroaryl-CO—, or optionally substituted 9-10 membered heteroaryl-CO— is optionally substituted by 1 or 2 substituents independently selected from halogen, cyano, (C₁-C₄)alkyl, (C₁-C₄)alkoxy, (C₁-C₄)alkyl-CO—, halo(C₁-C₄)alkyl, halo(C₁-C₄)alkyl-CO—, (C₃-C₆)cycloalkyl and 5-6 membered heterocycloalkyl; or
   said substituted (C₂-C₄)alkynyl, (C₃-C₆)cycloalkyl or 5-6 membered heterocycloalkyl group is substituted by an optionally substituted phenyl, 5-6 membered heteroaryl or 9-membered heteroaryl group,
      wherein said phenyl, 5-6 membered heteroaryl or 9-membered heteroaryl group is optionally substituted by 1 or 2 substituents independently selected from halogen, (C₁-C₄)alkyl, (C₁-C₄)alkyl-CO—, halo(C₁-C₄)alkyl, and halo(C₁-C₄)alkyl-CO—;
         R² is a substituted or unsubstituted phenyl, (C₃-C₆)cycloalkyl, 5-6 membered oxygen-containing heterocycloalkyl, 5-6 membered heteroaryl, 9-membered heteroaryl, 9-10 membered carbocyclic-aryl, or 9-10 membered heterocyclic-aryl group,
wherein said substituted phenyl, (C₃-C₆)cycloalkyl, 5-6 membered heterocycloalkyl, 5-6 membered heteroaryl, 9-membered heteroaryl, 9-10 membered carbocyclic-aryl, or 9-10 membered heterocyclic-aryl group is substituted by 1, 2 or 3 substituents independently selected from halogen, (C₁-C₄)alkyl, halo(C₁-C₄)alkyl, (C₁-C₄)alkoxy, halo(C₁-C₄)alkoxy, and cyano; and
   R³ is H or halogen;
or a salt, particularly a pharmaceutically acceptable salt, thereof.

In one embodiment, a compound disclosed in U.S. Provisional Patent Application No. 62/424,047, filed Nov. 18, 2016 (and U.S. Provisional Patent Application No. 62/585,267, filed Nov. 13, 2017, the disclosure of each of which is incorporated by reference herein), that inhibits RIP1 kinase is a compound having the following Formula:

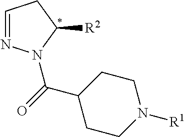

wherein:
R¹ is a substituted or unsubstituted 5-6 membered heteroaryl or 9-10 membered heteroaryl group,
wherein said substituted 5-6 membered heteroaryl or 9-10 membered heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl, H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)N—CO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
   wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and
      R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano;
   or a pharmaceutically acceptable salt thereof.

These compounds may be prepared according to Scheme 1, Scheme 2, Scheme 3, Scheme 4, or analogous methods. Wittig reaction of an aryl aldehyde of Formula A with (triphenylphosphoranylidene)-acetaldehyde affords an unsaturated aldehyde of Formula B. Reaction of an aldehyde of Formula B with hydrazine provides a dihydropyrazole of Formula C. The coupling of 1-(tert-butoxycarbonyl)piperidine-4-carboxylic acid with the dihydropyrazole of Formula C under amide bond forming conditions affords a compound of Formula D. Removal of the t-butoxycarbonyl group of a compound of Formula D affords a racemic piperdine of Formula E. Treatment of the racemic piperidine of Formula E with a chiral acid (e.g. (1R)-(−)-10-camphorsulfonic acid) provides a chiral amine salt of Formula F. Reaction of a compound of Formula F with and aryl halide or aryl sulfone under nucleophilic aromatic substitution conditions provides a compound having the above formula.

Alternatively, these compounds can be prepared through further transformation of a preexisting functional group. For example, as in Scheme 2, a compound possessing a carboxylate ester (Formula G) may be hydrolyzed to provide a new compound possessing a carboxylic acid (Formula H). Additionally, a compound of Formula H may be further transformed through an amide bond forming reaction to afford an alternate compound of possessing an amide (Formula J).

Alternatively, a compound can be prepared from a compound of Formula J according to Scheme 3. Reaction of the primary amide of a compound of Formula J with phosphorous oxychloride provides a compound possessing a nitrile (Formula K).

Alternatively, a compound may be prepared from another compound possessing a preexisting halogen (Formula L) according to Scheme 4. Reaction of a compound of Formula L with a primary or secondary amine under nucleophilic aromatic substitution conditions provides a compound of Formula M.

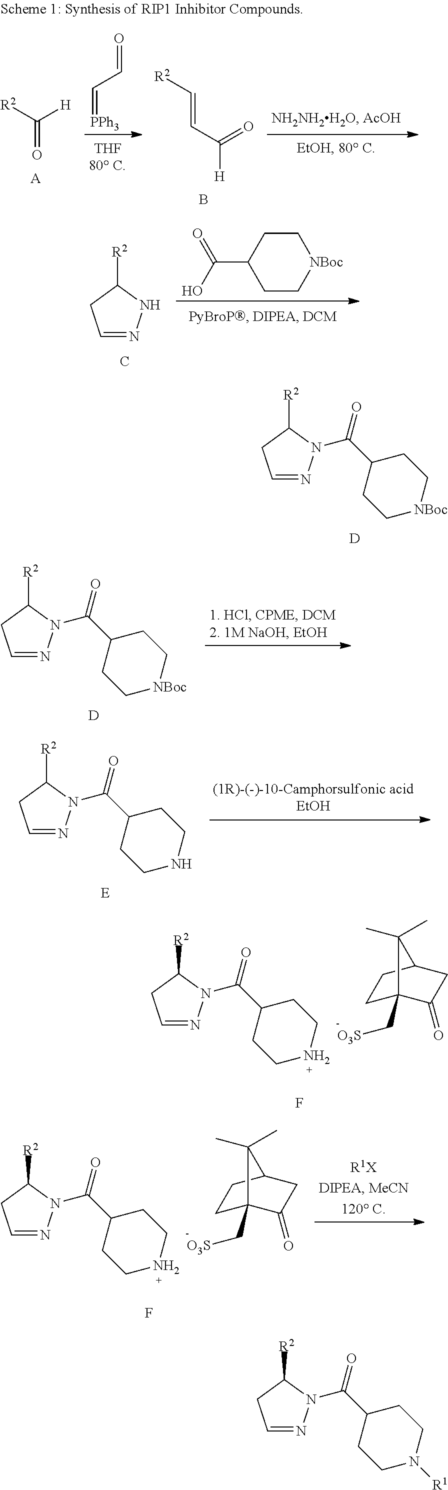

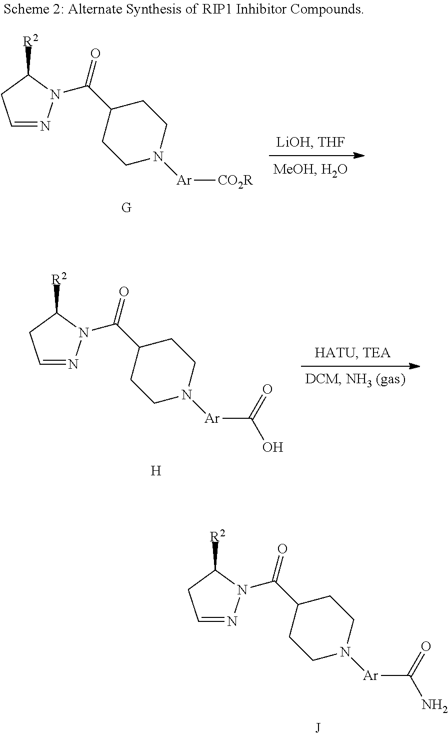

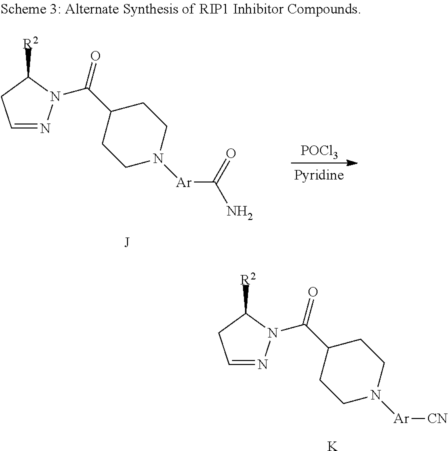

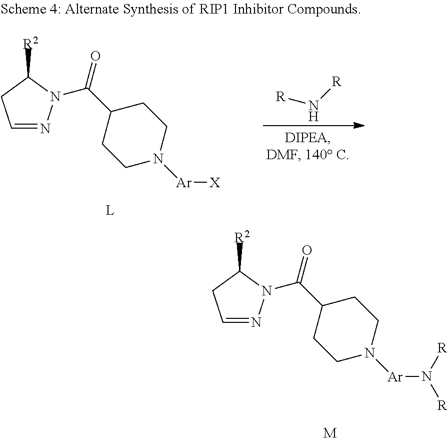

In one embodiment, a compound that inhibits RIP1 kinase is:
(S)-(1-(4-(benzylthio)pyrimidin-2-yl)piperidin-4-yl)(5-phenyl-4,5-dihydro-1H-pyrazol-1-yl)methanone;
2-(4-(5-phenyl-4,5-dihydro-1H-pyrazole-1-carbonyl)piperidin-1-yl)pyrimidine-5-carbonitrile;
(1-(4-methoxyl)pyrimidin-2-yl)piperidin-4-yl)(5-phenyl-4,5-dihydro-1H-pyrazol-1-yl)methanone;
(5-phenyl-4,5-dihydro-1H-pyrazol-1-yl)(1-(4-phenylpyrimidin-2-yl)piperidin-4-yl)methanone;
2-(4-(5-phenyl-4,5-dihydro-1H-pyrazole-1-carbonyl)piperidin-1-yl)pyrimidine-4-carbonitrile;
(1-(4-aminopyrimidin-2-yl)piperidin-4-yl)(5-phenyl-4,5-dihydro-1H-pyrazol-1-yl)methanone;
(1-(5-methoxyl)pyrimidin-2-yl)piperidin-4-yl)(5-phenyl-4,5-dihydro-1H-pyrazol-1-yl)methanone;
(S)-(1-(5-(methylsulfonyl)pyrimidin-2-yl)piperidin-4-yl)(5-phenyl-4,5-dihydro-1H-pyrazol-1-yl)methanone;
(S)-(1-(7H-purin-2-yl)piperidin-4-yl)(5-phenyl-4,5-dihydro-1H-pyrazol-1-yl)methanone;
(S)-methyl 2-(4-(5-phenyl-4,5-dihydro-1H-pyrazole-1-carbonyl)piperidin-1-yl)pyrimidine-5-carboxylate;
(S)-(1-(2-aminopyrimidin-4-yl)piperidin-4-yl)(5-phenyl-4,5-dihydro-1H-pyrazol-1-yl)methanone;
(S)-(1-(6-methoxyl)pyrimidin-4-yl)piperidin-4-yl)(5-phenyl-4,5-dihydro-1H-pyrazol-1-yl)methanone;
(S)-(1-(5-methoxyl)pyrimidin-2-yl)piperidin-4-yl)(5-phenyl-4,5-dihydro-1H-pyrazol-1-yl)methanone;
(S)-6-(4-(5-phenyl-4,5-dihydro-1H-pyrazole-1-carbonyl)piperidin-1-yl)pyrimidine-4-carbonitrile;
(S)-(1-(2-(methylamino)pyrimidin-4-yl)piperidin-4-yl)(5-phenyl-4,5-dihydro-1H-pyrazol-1-yl)methanone;
(S)-(1-(4-(methylamino)pyrimidin-2-yl)piperidin-4-yl)(5-phenyl-4,5-dihydro-1H-pyrazol-1-yl)methanone;
(S)-(1-(2-methoxyl)pyrimidin-4-yl)piperidin-4-yl)(5-phenyl-4,5-dihydro-1H-pyrazol-1-yl)methanone;
(S)-4-(4-(5-phenyl-4,5-dihydro-1H-pyrazole-1-carbonyl)piperidin-1-yl)pyrimidine-2-carbonitrile;
(S)-2-(4-(5-phenyl-4,5-dihydro-1H-pyrazole-1-carbonyl)piperidin-1-yl)pyrimidin-4(3H)-one;
(S)-(1-(6-aminopyrimidin-4-yl)piperidin-4-yl)(5-phenyl-4,5-dihydro-1H-pyrazol-1-yl)methanone;
(S)-(5-phenyl-4,5-dihydro-1H-pyrazol-1-yl)(1-(pyrazolo[1,5-a]pyrimidin-5-yl)piperidin-4-yl)methanone;
(S)-(1-(imidazo[1,2-b]pyridazin-6-yl)piperidin-4-yl)(5-phenyl-4,5-dihydro-1H-pyrazol-1-yl)methanone;
(S)-(1-(9-methyl-9H-purin-2-yl)piperidin-4-yl)(5-phenyl-4,5-dihydro-1H-pyrazol-1-yl)methanone;
(S)-2-(4-(5-phenyl-4,5-dihydro-1H-pyrazole-1-carbonyl)piperidin-1-yl)-5H-pyrrolo[2,3-d]pyrimidin-6(7H)-one;
(S)-6-(4-(5-phenyl-4,5-dihydro-1H-pyrazole-1-carbonyl)piperidin-1-yl)pyridazine-3-carboxamide;
(S)-(5-phenyl-4,5-dihydro-1H-pyrazol-1-yl)(1-(6-(trifluoromethyl)pyridazin-3-yl)piperidin-4-yl)methanone;
(S)-(1-(4-amino-5-fluoropyrimidin-2-yl)piperidin-4-yl)(5-phenyl-4,5-dihydro-1H-pyrazol-1-yl)methanone;
(S)-2-(4-(5-phenyl-4,5-dihydro-1H-pyrazole-1-carbonyl)piperidin-1-yl)pyrimidine-4-carboxamide;
(S)-2-(4-(5-phenyl-4,5-dihydro-1H-pyrazole-1-carbonyl)piperidin-1-yl)pyrimidine-4-carboxylic acid;
(S)-6-(4-(5-phenyl-4,5-dihydro-1H-pyrazole-1-carbonyl)piperidin-1-yl)nicotinamide;
(S)-6-(4-(5-phenyl-4,5-dihydro-1H-pyrazole-1-carbonyl)piperidin-1-yl)pyrazine-2-carboxamide;
(S)-6-(4-(5-phenyl-4,5-dihydro-1H-pyrazole-1-carbonyl)piperidin-1-yl)pyrimidine-4-carboxamide;
(S)-(1-(6-amino-2-methylpyrimidin-4-yl)piperidin-4-yl)(5-phenyl-4,5-dihydro-1H-pyrazol-1-yl)methanone;
(S)-(1-(2-amino-6-methoxyl)pyrimidin-4-yl)piperidin-4-yl)(5-phenyl-4,5-dihydro-1H-pyrazol-1-yl)methanone;
(S)-(1-(6-amino-2-methoxyl)pyrimidin-4-yl)piperidin-4-yl)(5-phenyl-4,5-dihydro-1H-pyrazol-1-yl)methanone;
(S)—N-(2-(4-(5-phenyl-4,5-dihydro-1H-pyrazole-1-carbonyl)piperidin-1-yl)pyrimidin-4-yl)acetamide;
(S)-6-(4-(5-phenyl-4,5-dihydro-1H-pyrazole-1-carbonyl)piperidin-1-yl)-1H-pyrazolo[3,4-d]pyrimidin-4(7H)-one;
(S)-(5-phenyl-4,5-dihydro-1H-pyrazol-1-yl)(1-(6-phenylpyrazin-2-yl)piperidin-4-yl)methanone;
(S)-(5-phenyl-4,5-dihydro-1H-pyrazol-1-yl)(1-(quinoxalin-2-yl)piperidin-4-yl)methanone;
(S)-5-(4-(5-phenyl-4,5-dihydro-1H-pyrazole-1-carbonyl)piperidin-1-yl)pyrazine-2-carbonitrile;
(S)-(1-(6-aminopyrazin-2-yl)piperidin-4-yl)(5-phenyl-4,5-dihydro-1H-pyrazol-1-yl)methanone;
(S)-6-(4-(5-phenyl-4,5-dihydro-1H-pyrazole-1-carbonyl)piperidin-1-yl)pyridazine-3-carbonitrile;
(S)-(1-(6-hydroxyl)pyrimidin-4-yl)piperidin-4-yl)(5-phenyl-4,5-dihydro-1H-pyrazol-1-yl)methanone;
(S)-3-(4-(5-phenyl-4,5-dihydro-1H-pyrazole-1-carbonyl)piperidin-1-yl)pyrazine-2-carbonitrile;
(S)-(1-(2-(methylthio)pyrimidin-4-yl)piperidin-4-yl)(5-phenyl-4,5-dihydro-1H-pyrazol-1-yl)methanone;
(S)-(5-phenyl-4,5-dihydro-1H-pyrazol-1-yl)(1-(2-(trifluoromethyl)pyrimidin-4-yl)piperidin-4-yl)methanone;
(S)-6-(4-(5-phenyl-4,5-dihydro-1H-pyrazole-1-carbonyl)piperidin-1-yl)pyrazine-2-carbonitrile;
(S)-(1-(6-methoxyl)pyrazin-2-yl)piperidin-4-yl)(5-phenyl-4,5-dihydro-1H-pyrazol-1-yl)methanone;
(S)-(1-(6-methoxyl)pyridazin-3-yl)piperidin-4-yl)(5-phenyl-4,5-dihydro-1H-pyrazol-1-yl)methanone;
(S)-4-(4-(5-phenyl-4,5-dihydro-1H-pyrazole-1-carbonyl)piperidin-1-yl)pyrimidine-5-carbonitrile;
(S)-(5-phenyl-4,5-dihydro-1H-pyrazol-1-yl)(1-(6-(trifluoromethyl)pyrimidin-4-yl)piperidin-4-yl)methanone;
(S)-(1-(1H-pyrazolo[3,4-d]pyrimidin-6-yl)piperidin-4-yl)(5-phenyl-4,5-dihydro-1H-pyrazol-1-yl)methanone;
(S)-2-(4-(5-phenyl-4,5-dihydro-1H-pyrazole-1-carbonyl)piperidin-1-yl)thiazole-4-carbonitrile;
(S)—N-methyl-2-(4-(5-phenyl-4,5-dihydro-1H-pyrazole-1-carbonyl)piperidin-1-yl)thiazole-4-carboxamide;
(S)-2-(4-(5-phenyl-4,5-dihydro-1H-pyrazole-1-carbonyl)piperidin-1-yl)thiazole-5-carboxamide;
(S)-2-(4-(5-phenyl-4,5-dihydro-1H-pyrazole-1-carbonyl)piperidin-1-yl)thiazole-4-carboxamide;
(S)-(1-(5-phenyl-1,3,4-oxadiazol-2-yl)piperidin-4-yl)(5-phenyl-4,5-dihydro-1H-pyrazol-1-yl)methanone;
(S)-(1-(4-ethoxyl)pyrimidin-2-yl)piperidin-4-yl)(5-phenyl-4,5-dihydro-1H-pyrazol-1-yl)methanone;
(S)-(1-(6-(methylthio)pyrimidin-4-yl)piperidin-4-yl)(5-phenyl-4,5-dihydro-1H-pyrazol-1-yl)methanone;
(S)-(1-(6-amino-2-(methyl thio)pyrimidin-4-yl)piperidin-4-yl)(5-phenyl-4,5-dihydro-1H-pyrazol-1-yl)methanone;
(S)-(1-(6-amino-5-fluoropyrimidin-4-yl)piperidin-4-yl)(5-phenyl-4,5-dihydro-1H-pyrazol-1-yl)methanone;
(S)-3-(1-(1-(pyrazolo[1,5-a]pyrimidin-5-yl)piperidine-4-carbonyl)-4,5-dihydro-1H-pyrazol-5-yl)benzonitrile;
(S)-5-(4-(5-phenyl-4,5-dihydro-1H-pyrazole-1-carbonyl)piperidin-1-yl)pyrazine-2-carboxamide;
(S)—N-(6-(4-(5-phenyl-4,5-dihydro-1H-pyrazole-1-carbonyl)piperidin-1-yl)pyrazin-2-yl)acetamide;
(S)-ethyl 2-(4-(5-phenyl-4,5-dihydro-1H-pyrazole-1-carbonyl)piperidin-1-yl)oxazole-4-carboxylate;
(S)-ethyl 2-(4-(5-phenyl-4,5-dihydro-1H-pyrazole-1-carbonyl)piperidin-1-yl)oxazole-5-carboxylate;
(S)-(5-phenyl-4,5-dihydro-1H-pyrazol-1-yl)(1-(5-phenyloxazol-2-yl)piperidin-4-yl)methanone;
(S)-6-(4-(5-(3-cyanophenyl)-4,5-dihydro-1H-pyrazole-1-carbonyl)piperidin-1-yl)pyrimidine-4-carbonitrile;
(S)-3-(1-(1-(4-methoxyl)pyrimidin-2-yl)piperidine-4-carbonyl)-4,5-dihydro-1H-pyrazol-5-yl)benzonitrile;
(S)-6-(4-(5-(3-cyanophenyl)-4,5-dihydro-1H-pyrazole-1-carbonyl)piperidin-1-yl)pyrimidine-4-carboxamide;
(S)-2-(4-(5-(3-cyanophenyl)-4,5-dihydro-1H-pyrazole-1-carbonyl)piperidin-1-yl)pyrimidine-4-carboxamide;
(S)-3-(1-(1-(4-amino-5-fluoropyrimidin-2-yl)piperidine-4-carbonyl)-4,5-dihydro-1H-pyrazol-5-yl)benzonitrile;
(S)-3-(1-(1-(imidazo[1,2-b]pyridazin-6-yl)piperidine-4-carbonyl)-4,5-dihydro-1H-pyrazol-5-yl)benzonitrile;
(S)-4-(4-(5-(3-cyanophenyl)-4,5-dihydro-1H-pyrazole-1-carbonyl)piperidin-1-yl)pyrimidine-2-carbonitrile;
(S)-3-(1-(1-(2-methoxyl)pyrimidin-4-yl)piperidine-4-carbonyl)-4,5-dihydro-1H-pyrazol-5-yl)benzonitrile;
(S)-3-(1-(1-(1H-pyrazolo[3,4-d]pyrimidin-6-yl)piperidine-4-carbonyl)-4,5-dihydro-1H-pyrazol-5-yl)benzonitrile;
(S)-(5-(3,5-difluorophenyl)-4,5-dihydro-1H-pyrazol-1-yl)(1-(5-methyl-1,3,4-oxadiazol-2-yl)piperidin-4-yl)methanone;
(S)-6-(4-(5-(3,5-difluorophenyl)-4,5-dihydro-1H-pyrazole-1-carbonyl)piperidin-1-yl)pyrimidine-4-carbonitrile;
(S)-(5-(3,5-difluorophenyl)-4,5-dihydro-1H-pyrazol-1-yl)(1-(4-methoxyl)pyrimidin-2-yl)piperidin-4-yl)methanone;
(S)-2-(4-(5-(3,5-difluorophenyl)-4,5-dihydro-1H-pyrazole-1-carbonyl)piperidin-1-yl)-5H-pyrrolo[2,3-d]pyrimidin-6(7H)-one;
(S)-(1-(4-amino-5-fluoropyrimidin-2-yl)piperidin-4-yl)(5-(3,5-difluorophenyl)-4,5-dihydro-1H-pyrazol-1-yl)methanone;
(S)-(5-(3,5-difluorophenyl)-4,5-dihydro-1H-pyrazol-1-yl)(1-(2-(methylthio)pyrimidin-4-yl)piperidin-4-yl)methanone;
(S)-2-(4-(5-(3,5-difluorophenyl)-4,5-dihydro-1H-pyrazole-1-carbonyl)piperidin-1-yl)pyrimidine-4-carboxamide;
(S)-(1-(2-aminopyrimidin-4-yl)piperidin-4-yl)(5-(3,5-difluorophenyl)-4,5-dihydro-1H-pyrazol-1-yl)methanone;
(S)-6-(4-(5-(3,5-difluorophenyl)-4,5-dihydro-1H-pyrazole-1-carbonyl)piperidin-1-yl)pyrimidine-4-carboxamide;
(S)-(1-(1H-pyrazolo[3,4-d]pyrimidin-6-yl)piperidin-4-yl)(5-(3,5-difluorophenyl)-4,5-dihydro-1H-pyrazol-1-yl)methanone;
(S)-(5-(3,5-difluorophenyl)-4,5-dihydro-1H-pyrazol-1-yl)(1-(imidazo[1,2-b]pyridazin-6-yl)piperidin-4-yl)methanone;
(S)-4-(4-(5-(3,5-difluorophenyl)-4,5-dihydro-1H-pyrazole-1-carbonyl)piperidin-1-yl)pyrimidine-5-carbonitrile;
(S)-4-(4-(5-(3,5-difluorophenyl)-4,5-dihydro-1H-pyrazole-1-carbonyl)piperidin-1-yl)pyrimidine-2-carbonitrile;
(S)-(5-(3,5-difluorophenyl)-4,5-dihydro-1H-pyrazol-1-yl)(1-(pyrazolo[1,5-a]pyrimidin-5-yl)piperidin-4-yl)methanone;
(S)-(5-(3,5-difluorophenyl)-4,5-dihydro-1H-pyrazol-1-yl)(1-(6-methoxyl)pyrimidin-4-yl)piperidin-4-yl)methanone;
(S)-2-(4-(5-(3,5-difluorophenyl)-4,5-dihydro-1H-pyrazole-1-carbonyl)piperidin-1-yl)pyrimidine-5-carbonitrile;
(S)-(1-(4-aminopyrimidin-2-yl)piperidin-4-yl)(5-(3,5-difluorophenyl)-4,5-dihydro-1H-pyrazol-1-yl)methanone;
(S)-6-(4-(5-(3,5-difluorophenyl)-4,5-dihydro-1H-pyrazole-1-carbonyl)piperidin-1-yl)pyridazine-3-carbonitrile;
(S)-5-(4-(5-(3,5-difluorophenyl)-4,5-dihydro-1H-pyrazole-1-carbonyl)piperidin-1-yl)pyrazine-2-carbonitrile;
(S)-2-(4-(5-(3,5-difluorophenyl)-4,5-dihydro-1H-pyrazole-1-carbonyl)piperidin-1-yl)pyrimidine-5-carboxamide;
(S)-2-(4-(5-(3,5-difluorophenyl)-4,5-dihydro-1H-pyrazole-1-carbonyl)piperidin-1-yl)pyrimidin-4(3H)-one;
(S)-(1-(6-aminopyrimidin-4-yl)piperidin-4-yl)(5-(3,5-difluorophenyl)-4,5-dihydro-1H-pyrazol-1-yl)methanone;
(S)-(5-(3,5-difluorophenyl)-4,5-dihydro-1H-pyrazol-1-yl)(1-(2-methoxyl)pyrimidin-4-yl)piperidin-4-yl)methanone;
(S)-(5-(3,5-difluorophenyl)-4,5-dihydro-1H-pyrazol-1-yl)(1-(2-(methylamino)pyrimidin-4-yl)piperidin-4-yl)methanone;
(S)-(5-(2,5-difluorophenyl)-4,5-dihydro-1H-pyrazol-1-yl)(1-(5-methoxyl)pyrimidin-2-yl)piperidin-4-yl)methanone,
(S)-2-(4-(5-(2,5-difluorophenyl)-4,5-dihydro-1H-pyrazole-1-carbonyl)piperidin-1-yl)pyrimidine-4-carboxamide;
(S)-5-(4-(5-(2,5-difluorophenyl)-4,5-dihydro-1H-pyrazole-1-carbonyl)piperidin-1-yl)pyrazine-2-carbonitrile;
(S)-6-(4-(5-(2,5-difluorophenyl)-4,5-dihydro-1H-pyrazole-1-carbonyl)piperidin-1-yl)pyrimidine-4-carbonitrile;
(S)-(5-(2,5-difluorophenyl)-4,5-dihydro-1H-pyrazol-1-yl)(1-(6-methoxyl)pyrimidin-4-yl)piperidin-4-yl)methanone;
(S)-ethyl 2-(4-(5-(3,5-difluorophenyl)-4,5-dihydro-1H-pyrazole-1-carbonyl)piperidin-1-yl)oxazole-4-carboxylate;
(S)-ethyl 2-(4-(5-(3,5-difluorophenyl)-4,5-dihydro-1H-pyrazole-1-carbonyl)piperidin-1-yl)oxazole-5-carboxylate;
(S)-6-(4-(5-(5-fluoropyridin-3-yl)-4,5-dihydro-1H-pyrazole-1-carbonyl)piperidin-1-yl)pyrimidine-4-carboxamide;
(S)-2-(4-(5-(5-fluoropyridin-3-yl)-4,5-dihydro-1H-pyrazole-1-carbonyl)piperidin-1-yl)pyrimidine-4-carboxamide;
(S)-(5-(5-fluoropyridin-3-yl)-4,5-dihydro-1H-pyrazol-1-yl)(1-(2-methoxyl)pyrimidin-4-yl)piperidin-4-yl)methanone;
(S)-6-(4-(5-(5-fluoropyridin-3-yl)-4,5-dihydro-1H-pyrazole-1-carbonyl)piperidin-1-yl)pyrimidine-4-carbonitrile;
(S)-(1-(4-amino-5-fluoropyrimidin-2-yl)piperidin-4-yl)(5-(5-fluoropyridin-3-yl)-4,5-dihydro-1H-pyrazol-1-yl)methanone;
(S)-(5-(5-fluoropyridin-3-yl)-4,5-dihydro-1H-pyrazol-1-yl)(1-(6-methoxyl)pyrimidin-4-yl)piperidin-4-yl)methanone;
(S)-(5-(5-fluoropyridin-3-yl)-4,5-dihydro-1H-pyrazol-1-yl)(1-(2-(methylthio)pyrimidin-4-yl)piperidin-4-yl)methanone;
(S)-4-(4-(5-(5-fluoropyridin-3-yl)-4,5-dihydro-1H-pyrazole-1-carbonyl)piperidin-1-yl)pyrimidine-2-carbonitrile;
(S)-(5-(5-fluoropyridin-3-yl)-4,5-dihydro-1H-pyrazol-1-yl)(1-(pyrazolo[1,5-a]pyrimidin-5-yl)piperidin-4-yl)methanone;
(S)-(5-(5-fluoropyridin-3-yl)-4,5-dihydro-1H-pyrazol-1-yl)(1-(imidazo[1,2-b]pyridazin-6-yl)piperidin-4-yl)methanone;
(S)—N-(2-(4-(5-(2,5-difluorophenyl)-4,5-dihydro-1H-pyrazole-1-carbonyl)piperidin-1-yl)pyrimidin-4-yl)acetamide;
(S)—N-(6-(4-(5-phenyl-4,5-dihydro-1H-pyrazole-1-carbonyl)piperidin-1-yl)pyrimidin-4-yl)acetamide;
(S)—N-(6-(4-(5-(3,5-difluorophenyl)-4,5-dihydro-1H-pyrazole-1-carbonyl)piperidin-1-yl)pyrimidin-4-yl)acetamide;
(S)-(1-(5-fluoro-4-(4-methylpiperazin-1-yl)pyrimidin-2-yl)piperidin-4-yl)(5-phenyl-4,5-dihydro-1H-pyrazol-1-yl)methanone;
(S)-(1-(2-(dimethylamino)pyrimidin-4-yl)piperidin-4-yl)(5-phenyl-4,5-dihydro-1H-pyrazol-1-yl)methanone;
(S)-2-(4-(5-(2,5-difluorophenyl)-4,5-dihydro-1H-pyrazole-1-carbonyl)piperidin-1-yl)pyrimidine-5-carboxamide;
(S)-5-chloro-2-(4-(5-(5-fluoropyridin-3-yl)-4,5-dihydro-1H-pyrazole-1-carbonyl)piperidin-1-yl)pyrimidine-4-carboxamide;
(S)—N-cyclopropyl-2-(4-(5-phenyl-4,5-dihydro-1H-pyrazole-1-carbonyl)piperidin-1-yl)pyrimidine-4-carboxamide;
(S)—N-(2-hydroxyethyl)-2-(4-(5-phenyl-4,5-dihydro-1H-pyrazole-1-carbonyl)piperidin-1-yl)pyrimidine-4-carboxamide;
(S)-(1-(5-fluoro-4-(2-morpholinoethoxy)pyrimidin-2-yl)piperidin-4-yl)(5-phenyl-4,5-dihydro-1H-pyrazol-1-yl)methanone;
(S)-(1-(5-hydroxyl)pyrimidin-2-yl)piperidin-4-yl)(5-phenyl-4,5-dihydro-1H-pyrazol-1-yl)methanone;
(S)-(1-(6-(5-methyl-1,3,4-oxadiazol-2-yl)pyrimidin-4-yl)piperidin-4-yl)(5-phenyl-4,5-dihydro-1H-pyrazol-1-yl)methanone;
(S)-(1-(4-(hydroxymethyl)pyrimidin-2-yl)piperidin-4-yl)(5-phenyl-4,5-dihydro-1H-pyrazol-1-yl)methanone;
(S)-2-(4-(5-phenyl-4,5-dihydro-1H-pyrazole-1-carbonyl)piperidin-1-yl)pyrimidine-5-carboxamide;
(S)-2-(4-(5-(3,5-difluorophenyl)-4,5-dihydro-1H-pyrazole-1-carbonyl)piperidin-1-yl)oxazole-5-carboxamide;
(S)-2-(4-(5-(3,5-difluorophenyl)-4,5-dihydro-1H-pyrazole-1-carbonyl)piperidin-1-yl)oxazole-5-carbonitrile;
(S)-2-(4-(5-(3,5-difluorophenyl)-4,5-dihydro-1H-pyrazole-1-carbonyl)piperidin-1-yl)oxazole-4-carboxamide;
(S)-2-(4-(5-(3,5-difluorophenyl)-4,5-dihydro-1H-pyrazole-1-carbonyl)piperidin-1-yl)oxazole-4-carbonitrile;
(S)-2-(4-(5-phenyl-4,5-dihydro-1H-pyrazole-1-carbonyl)piperidin-1-yl)oxazole-4-carboxamide;
(S)-2-(4-(5-phenyl-4,5-dihydro-1H-pyrazole-1-carbonyl)piperidin-1-yl)oxazole-4-carbonitrile;
(S)-2-(4-(5-phenyl-4,5-dihydro-1H-pyrazole-1-carbonyl)piperidin-1-yl)oxazole-5-carbonitrile;
(S)-(1-(7H-purin-2-yl)piperidin-4-yl)(5-(3,5-difluorophenyl)-4,5-dihydro-1H-pyrazol-1-yl)methanone;
(S)-(1-(4-(4,5-dihydro-1H-imidazol-2-yl)pyrimidin-2-yl)piperidin-4-yl)(5-phenyl-4,5-dihydro-1H-pyrazol-1-yl)methanone, (S)-(4-methylpiperazin-1-yl)(2-(4-(5-phenyl-4,5-dihydro-1H-pyrazole-1-carbonyl)piperidin-1-yl)pyrimidin-4-yl)methanone;
(S)—N,N-diethyl-2-(4-(5-phenyl-4,5-dihydro-1H-pyrazole-1-carbonyl)piperidin-1-yl)pyrimidine-4-carboxamide;
(S)—N,N-dimethyl-2-(4-(5-phenyl-4,5-dihydro-1H-pyrazole-1-carbonyl)piperidin-1-yl)pyrimidine-4-carbohydrazide;
(S)—N-(1-acetylpiperidin-4-yl)-2-(4-(5-phenyl-4,5-dihydro-1H-pyrazole-1-carbonyl)piperidin-1-yl)pyrimidine-4-carboxamide;
(S)-(1-(4-(morpholine-4-carbonyl)pyrimidin-2-yl)piperidin-4-yl)(5-phenyl-4,5-dihydro-1H-pyrazol-1-yl)methanone;
(S)-(5-phenyl-4,5-dihydro-1H-pyrazol-1-yl)(1-(4-(piperazine-1-carbonyl)pyrimidin-2-yl)piperidin-4-yl)methanone;
(S)-2-(4-(5-phenyl-4,5-dihydro-1H-pyrazole-1-carbonyl)piperidin-1-yl)-N-(piperidin-4-yl)pyrimidine-4-carboxamide;
(S)-(1-(4-(2H-tetrazol-5-yl)pyrimidin-2-yl)piperidin-4-yl)(5-phenyl-4,5-dihydro-1H-pyrazol-1-yl)methanone;
(S)-(1-(4-(2H-tetrazol-5-yl)pyrimidin-2-yl)piperidin-4-yl)(5-(5-fluoropyridin-3-yl)-4,5-dihydro-1H-pyrazol-1-yl)methanone;
3-(5-fluoro-2-(4-((S)-5-phenyl-4,5-dihydro-1H-pyrazole-1-carbonyl)piperidin-1-yl)pyrimidin-4-yl)pyrrolidin-2-one;
3-(5-fluoro-2-(4-((S)-5-(5-fluoropyridin-3-yl)-4,5-dihydro-1H-pyrazole-1-carbonyl)piperidin-1-yl)pyrimidin-4-yl)pyrrolidin-2-one;
(S)-2-((2-(4-(5-phenyl-4,5-dihydro-1H-pyrazole-1-carbonyl)piperidin-1-yl)pyrimidin-4-yl)oxy)acetic acid;
(S)-(1-(4-((2H-tetrazol-5-yl)methoxy)-5-fluoropyrimidin-2-yl)piperidin-4-yl)(5-phenyl-4,5-dihydro-1H-pyrazol-1-yl)methanone;
(S)-(1-(5-fluoro-4-(2-hydroxyethoxy)pyrimidin-2-yl)piperidin-4-yl)(5-phenyl-4,5-dihydro-1H-pyrazol-1-yl)methanone;
(S)-(1-(6-ethynylpyrimidin-4-yl)piperidin-4-yl)(5-phenyl-4,5-dihydro-1H-pyrazol-1-yl)methanone;
(S)-(5-(3,5-difluorophenyl)-4,5-dihydro-1H-pyrazol-1-yl)(1-(6-ethynylpyrimidin-4-yl)piperidin-4-yl)methanone;
(S)-3-(1-(1-(6-ethynylpyrimidin-4-yl)piperidine-4-carbonyl)-4,5-dihydro-1H-pyrazol-5-yl)benzonitrile;
(S)-5-fluoro-6-(4-(5-phenyl-4,5-dihydro-1H-pyrazole-1-carbonyl)piperidin-1-yl)pyrimidine-4-carboxylic acid;
(S)-5-fluoro-6-(4-(5-(5-fluoropyridin-3-yl)-4,5-dihydro-1H-pyrazole-1-carbonyl)piperidin-1-yl)pyrimidine-4-carboxamide;
(S)-5-fluoro-2-(4-(5-(5-fluoropyridin-3-yl)-4,5-dihydro-1H-pyrazole-1-carbonyl)piperidin-1-yl)pyrimidine-4-carboxamide;
(S)-5-fluoro-2-(4-(5-phenyl-4,5-dihydro-1H-pyrazole-1-carbonyl)piperidin-1-yl)pyrimidine-4-carboxamide;
(S)—N,N-diethyl-5-fluoro-6-(4-(5-phenyl-4,5-dihydro-1H-pyrazole-1-carbonyl)piperidin-1-yl)pyrimidine-4-carboxamide;
(S)-6-(4-(5-(3,5-difluorophenyl)-4,5-dihydro-1H-pyrazole-1-carbonyl)piperidin-1-yl)-5-fluoropyrimidine-4-carboxylic acid;
(S)-6-(4-(5-(3,5-difluorophenyl)-4,5-dihydro-1H-pyrazole-1-carbonyl)piperidin-1-yl)-5-fluoropyrimidine-4-carboxamide;
(R)-3-(5-fluoro-2-(4-((S)-5-(5-fluoropyridin-3-yl)-4,5-dihydro-1H-pyrazole-1-carbonyl)piperidin-1-yl)pyrimidin-4-yl)pyrrolidin-2-one;
(S)-2-((5-fluoro-2-(4-(5-phenyl-4,5-dihydro-1H-pyrazole-1-carbonyl)piperidin-1-yl)pyrimidin-4-yl)oxy)acetamide;
(1-(4-(morpholin-3-yl)pyrimidin-2-yl)piperidin-4-yl)((S)-5-phenyl-4,5-dihydro-1H-pyrazol-1-yl)methanone;
(S)-2-(5-fluoro-2-(4-(5-phenyl-4,5-dihydro-1H-pyrazole-1-carbonyl)piperidin-1-yl)pyrimidin-4-yl)acetamide;
   or a pharmaceutically acceptable salt thereof.

In one embodiment, this invention is directed to a method of treating a RIP1 kinase-mediated disease or disorder which comprises administering a therapeutically effective amount of a compound that inhibits RIP1 kinase to a human in need thereof,
or a compound that inhibits RIP1 kinase for use in the treatment of a RIP1 kinase-mediated disease or disorder;
or the use of a compound that inhibits RIP1 kinase as an active therapeutic substance for the treatment of a RIP1 kinase-mediated disease or disorder;
or the use of a compound that inhibits RIP1 kinase in the manufacture of a medicament for the treatment of a RIP1 kinase-mediated disease or disorder;
wherein the RIP1 kinase-mediated disease or disorder is selected from pancreatic cancer, metastatic adenocarcinoma of the pancreas, pancreatic ductal adenocarcinoma, a malignancy of the endocrine cells in the pancreas, hepatocellular carcinoma, mesothelioma, melanoma, colorectal cancer, acute myeloid leukemia, metastasis, glioblastoma, breast cancer, gallbladder cancer, clear cell renal carcinoma, non-small cell lung carcinoma, and radiation induced necrosis.

In a specific embodiment, this invention is directed to a method comprising administering the compound that inhibits RIP1 kinase and at least one other therapeutically active agent.

As used herein the term “agent,” including a “therapeutically active agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

In another embodiment, the RIP1 kinase-mediated disease or disorder is selected from pancreatic cancer, metastatic adenocarcinoma of the pancreas, pancreatic ductal adenocarcinoma, a malignancy of the endocrine cells in the pancreas, hepatocellular carcinoma, mesothelioma, melanoma, colorectal cancer, acute myeloid leukemia, metastasis, glioblastoma, breast cancer, gallbladder cancer, clear cell renal carcinoma, non-small cell lung carcinoma, and radiation induced necrosis; and
the other therapeutically active agent is selected from sorafenib, gemcitabine, folinic acid, fluorouracil, irinotecan, oxaliplatin, capecitabine, doxorubicin, temozolomide, procarbazine, nitrosourea, a PARP inhibitor, an anti-her2 therapy, TDM-1, SERD, a VEGF inhibitor, a tyrosine kinase inhibitor, nab-paclitaxel, and antibodies to PD-1, PD-L1, OX40, ICOS, or CTLA4.

In another embodiment, the other therapeutically active agent is an immuno-modulator.

In another embodiment, the other therapeutically active agent is an antibody to PD-1, PD-L1, OX40, ICOS, or CTLA4.

In another embodiment, the RIP1 kinase-mediated disease or disorder is selected from pancreatic cancer, metastatic adenocarcinoma of the pancreas, pancreatic ductal adenocarcinoma, and a malignancy of the endocrine cells in the pancreas, and
the other therapeutically active agent is selected from gemcitabine, folinic acid, fluorouracil, irinotecan, oxaliplatin, nab-paclitaxel, and antibodies to PD-1, PD-L1, OX40, ICOS, or CTLA4.

“Treating” or “treatment” is intended to mean at least the mitigation of a disease or disorder in a patient. The methods of treatment for mitigation of a disease or disorder include the use of the compounds in this invention in any conventionally acceptable manner, for example for prevention, retardation, prophylaxis, therapy or cure of a RIP1 kinase-mediated disease or disorder, as described hereinabove. In reference to a particular condition, “treating” means: (1) to ameliorate the condition or one or more of the biological manifestations of the condition, (2) to interfere with (a) one or more points in the biological cascade that leads to or is responsible for the condition or (b) one or more of the biological manifestations of the condition (3) to alleviate one or more of the symptoms, effects or side effects associated with the condition or one or more of the symptoms, effects or side effects associated with the condition or treatment thereof, or (4) to slow the progression of the condition or one or more of the biological manifestations of the condition.

As used herein, “prevention” is understood to refer to the prophylactic administration of a drug to substantially diminish the likelihood or severity of a condition or biological manifestation thereof, or to delay the onset of such condition or biological manifestation thereof. The skilled artisan will appreciate that “prevention” is not an absolute term. Prophylactic therapy is appropriate, for example, when a subject is considered at high risk for developing cancer, such as when a subject has a strong family history of cancer or when a subject has been exposed to a carcinogen.

The compounds useful in this invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

A therapeutically “effective amount” is intended to mean that amount of a compound that, when administered to a patient in need of such treatment, is sufficient to effect treatment (e.g., an amount that will elicit the biological or medical response of a tissue, system, animal or human that is being sought). Thus, e.g., a therapeutically effective amount of a compound of Formula (I), (II), or (III), or a pharmaceutically acceptable salt thereof, is a quantity of an agent that, when administered to a human in need thereof, is sufficient to modulate and/or inhibit the activity of RIP1 kinase such that a disease condition which is mediated by that activity is reduced, alleviated or prevented. The term also includes within its scope amounts effective to enhance normal physiological function. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmacokinetic properties), disease or disorder and its severity and the specific composition and method being used, but can nevertheless be determined by one of skill in the art.

The administration of a therapeutically effective amount of the combinations of the invention (or therapeutically effective amounts of each of the components of the combination) are advantageous over the individual component compounds in that the combinations provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The compounds useful in this invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound useful in this invention depend on the pharmacokinetic properties of that compound, such as absorption, distribution, and half-life, which can be determined by the skilled artisan. In addition, suitable dosing regimens, including the duration such regimens are administered, for a compound useful in this invention depend on the disease or disorder being treated, the severity of the disease or disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Total daily dosages range from 1 mg to 2000 mg.

For use in therapy, the compounds useful in this invention will be normally, but not necessarily, formulated into a pharmaceutical composition prior to administration to a patient.

Accordingly, the invention also is directed to pharmaceutical compositions comprising a compound of Formula (I), (II), or (III), or a pharmaceutically acceptable salt thereof, and at least one pharmaceutically acceptable excipient. In one embodiment, there is provided a pharmaceutical composition comprising (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, and at least one pharmaceutically acceptable excipient. In another embodiment, there is provided a pharmaceutical composition comprising (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, or a pharmaceutically acceptable salt thereof, and at least one pharmaceutically acceptable excipient.

The compounds useful in this invention, particularly a compound that inhibits RIP1 kinase, particularly, the compounds of Formula (I), (II), or (III), or a pharmaceutically acceptable salt thereof, may be employed alone or in combination with one or more other therapeutic agents, e.g., pharmaceutically active compounds or biologic products (e.g., monoclonal antibodies). Combination therapies according to the present invention thus comprise the administration of at least one compound that inhibits RIP1 kinase, particularly a compound of Formula (I), (II), or (III), or a pharmaceutically acceptable salt thereof, and at least one other theraputically active agent. Preferably, combination therapies according to the present invention comprise the administration of at least one compound that inhibits RIP1 kinase, particularly a compound of Formula (I), (II), or (III), or a pharmaceutically acceptable salt thereof, and at least one other therapeutically active agent, specifically one or two other therapeutically active agents, more specifically one other therapeutically active agent. In the treatment of the above noted diseases and disorders, it will be understood that the other therapeutically active agent administered in combination with a compound of Formula (I), (II), or (III), or a pharmaceutically acceptable salt thereof, includes any agent that is considered as a “standard of care” therapy for that disease or disorder. Many of such standard of care therapies are described hereinbelow.

As used herein, “antigen binding protein” is any protein, including but not limited to antibodies, domains and other constructs described herein, that binds to an antigen, such as PD-1, PDL-1, OX-40, CTLA4 and/or ICOS. As used herein “antigen binding portion” of an antigen binding protein would include any portion of the antigen binding protein capable of binding to its target, including but not limited to, an antigen binding antibody fragment.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., V_{H}, V_{HH}, V_{L}, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing.

As used herein the term “agonist” refers to an antigen binding protein, for example an ICOS binding protein, which upon contact with its ligand or receptor causes one or more of the following (1) stimulates or activates the receptor, (2) enhances, increases or promotes, induces or prolongs an activity, function or presence of the ligand or receptor and/or (3) enhances, increases, promotes or induces the expression of the ligand or receptor. An “agonist” or activating antibody is one that enhances or initiates signaling by the antigen to which it binds. In some embodiments, agonist antibodies cause or activate signaling without the presence of the natural ligand. Agonist activity can be measured in vitro by various assays know in the art such as, but not limited to, measurement of cell signaling, cell proliferation, immune cell activation markers, cytokine production. Agonist activity can also be measured in vivo by various assays that measure surrogate end points such as, but not limited to the measurement of T cell proliferation or cytokine production. Thus, as used herein an “agonist antibody” is an antibody that upon contacting its target elicits at least one of the activities of an agonist. Agonist antibodies or antigen binding proteins of the present invention include, but are not limited to, agonist ICOS antibodies and agonist OX-40 antibodies.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen. The anti-PD-1, anti-PD-L1 antibodies of the invention block the signaling through PD-1 and restores a functional response by T-cells from a dysfunctional state to antigen stimulation. Anti-CTLA4 antibodies of the present invention, block inhibits TCR- and CD-28 mediated signal transduction. CTLA-4 engagement results in the inhibition of IL-2 synthesis and progression through the cell cycle and termination of T-cell responses. As a result, the antagonism of CTLA-4 (e.g., antagonist anti-CTLA antibodies) and or agonizing B7.1/B7.2/CD28 may be useful to enhance immune response in the treatment of infection (e.g., acute and chronic) and tumor immunity.

As used herein the term “cross competes for binding” refers to any binding protein that will compete for binding to it binding target with any of the binding proteins of the present invention. Competition for binding between two molecules for one target can be tested by various methods known in the art including Flow cytometry, Meso Scale Discovery and ELISA. Binding can be measured directly, meaning two or more binding proteins can be put in contact with the target or interest and binding may be measured for one or each. Alternatively, binding of molecules or interest can be tested against the binding or natural ligand and quantitatively compared with each other.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

The protein scaffold may be a derivative of a scaffold selected from the group consisting of CTLA-4, lipocalin, Protein A derived molecules such as Z-domain of Protein A (Affibody, SpA), A-domain (Avimer/Maxibody); heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin/adnectin; which has been subjected to protein engineering in order to obtain binding to an antigen, such as ICOS, other than the natural ligand.

Antigen binding site refers to a site on an antigen binding protein which is capable of specifically binding to an antigen, this may be a single variable domain, or it may be paired V_{H}/V_{L} domains as can be found on a standard antibody. Single-chain Fv (ScFv) domains can also provide antigen-binding sites. The term “epitope-binding domain” refers to a domain that specifically binds to a region of an antigen known as the epitope independently of a different domain.

The term multi-specific antigen binding protein refers to antigen binding proteins which comprise at least two different antigen binding sites. Each of these antigen-binding sites will be capable of binding to a different epitope, which may be present on the same antigen or different antigens. The multi-specific antigen binding protein will have specificity for more than one antigen, for example two antigens, or for three antigens, or for four antigens.

The subclass of an antibody in part determines secondary effector functions, such as complement activation or Fc receptor (FcR) binding and antibody dependent cell cytotoxicity (ADCC) (Huber, et al., Nature 229(5284): 419-20 (1971); Brunhouse, et al., Mol Immunol 16(11): 907-17 (1979)). In identifying the optimal type of antibody for a particular application, the effector functions of the antibodies can be taken into account. For example, hIgG1 antibodies have a relatively long half life, are very effective at fixing complement, and they bind to both FcγRI and FcγRII. In contrast, human IgG4 antibodies have a shorter half life, do not fix complement and have a lower affinity for the FcRs. Replacement of serine 228 with a proline (S228P) in the Fc region of IgG4 reduces heterogeneity observed with hIgG4 and extends the serum half life (Kabat, et al., “Sequences of proteins of immunological interest” 5.sup.th Edition (1991); Angal, et al., Mol Immunol 30(1): 105-8 (1993)). A second mutation that replaces leucine 235 with a glutamic acid (L235E) eliminates the residual FcR binding and complement binding activities (Alegre, et al., J Immunol 148(11): 3461-8 (1992)). The resulting antibody with both mutations is referred to as IgG4PE. The numbering of the hIgG4 amino acids was derived from EU numbering reference: Edelman, G. M. et al., Proc. Natl. Acad. USA, 63, 78-85 (1969). PMID: 5257969. In one embodiment of the present invention ICOS antigen binding proteins comprising an IgG4 Fc region comprising the replacement S228P and L235E may have the designation IgG4PE. Thus, an ICOS binding protein having the heavy chain variable region H₂ and the light chain variable region L5 and an IgG4PE Fc region will be designated as H₂L5 IgG4PE or synonymously as H₂L5 hIgG4PE.

As used herein “immuno-modulators” refer to any substance including, but not limited to, antigen binding proteins and monoclonal antibodies that affects the immune system. Immuno-modulators can be used as anti-neoplastic agents for the treatment of cancer. Therefore, “immuno-modulators” are therapeutically active agents. For example, immuno-modulators include, but are not limited to, anti-CTLA-4 antibodies such as ipilimumab (YERVOY); anti-PD-1 antibodies (Opdivo/nivolumab and Keytruda/pembrolizumab); anti-PD-L1 antibodies ((TECENTRIQ (atezolizumab) IMFINZI (durvalumumab) and BAVENCIO (avelumab)). Other immuno-modulators include, but are not limited to, PD-1 antibodies, CTLA4 antibodies; ICOS antibodies, OX-40 antibodies, PD-L1 antibodies, LAG3 antibodies, TIM-3 antibodies, 41BB antibodies and GITR antibodies.

Immuno-modulators may include any agents that block the interaction between PD-1 and PD-L1, including, but not limited to antibodies directed to PD-1 and/or PDL1. In one aspect, the immuno-modulator is an anti-PD-L1 antibody. Anti-PD-L1 antibodies and methods of making the same are known in the art. Such antibodies to PD-L1 may be polyclonal or monoclonal, and/or recombinant, and/or humanized or fully human. Exemplary PD-L1 antibodies are disclosed in U.S. Pat. Nos. 8,217,149, 8,383,796, 8,552,154, 9,212,224, and 8,779,108, and US Patent Appln. Pub. Nos. 20110280877, 2014/0341902 and 20130045201. Additional exemplary antibodies to PD-L1 (also referred to as CD274 or B7-H₁) and methods for use are disclosed in U.S. Pat. Nos. 7,943,743, 8,168,179; and 7,595,048, WO2014055897, WO2016007235 and US Patent Appln. Pub. Nos. 20130034559, 20130034559 and 20150274835. PD-L1 antibodies are in development as immuno-modulatory agents or immuno-modulators for the treatment of cancer. TECENTRIQ (atezolizumab) is a PD-L1 antibody approved for the treatment of people with metastatic non-small cell lung cancer (NSCLC) who have disease progression during or following platinum-containing chemotherapy, and have progressed on an appropriate FDA-approved targeted therapy if their tumor has EGFR or ALK gene abnormalities. IMFINZI (durvalumumab) is an antibody PD-L1 antibody that blocks the interaction of PD-L1 with PD-1 and CD80.

In one embodiment, the antibody to PD-L1 is an antibody disclosed in U.S. Pat. No. 8,217,149. In another embodiment, the anti-PD-L1 antibody comprises the CDRs of an antibody disclosed in U.S. Pat. No. 8,217,149. In another embodiment, the antibody to PD-L1 is an antibody disclosed in U.S. Pat. No. 8,779,108. In another embodiment, the anti-PD-L1 antibody comprises the CDRs of an antibody disclosed in U.S. Pat. No. 8,779,108. In another embodiment, the antibody to PD-L1 is an antibody disclosed in US Patent Appln. Pub. No. 20130045201. In another embodiment, the anti-PD-L1 antibody comprises the CDRs of an antibody disclosed in US Patent Appln. Pub. No. 20130045201. In one embodiment, the anti-PD-L1 antibody is BMS-936559 (MDX-1105), which was described in WO 2007/005874. In another embodiment, the anti-PD-L1 antibody is MPDL3280A (RG7446). In another embodiment, the anti-PD-L1 antibody is MEDI4736, which is an anti-PD-L1 monoclonal antibody described in WO 2011/066389 and US 2013/034559. In another embodiment, the anti-PD-L1 antibody is TECENTRIQ™ (atezolizumab), which is an anti-PDL1 cancer immunotherapy which was approved in the US in May 2016 for specific types of bladder cancer. In another embodiment, anti-PD-L1 antibody is YW243.55.570 which is an anti-PD-L1 described in WO 2010/077634 and U.S. Pat. No. 8,217,149. Examples of anti-PD-L1 antibodies useful for the methods of this invention, and methods for making thereof are described in PCT patent application WO 2010/077634, WO 2007/005874, WO 2011/066389, U.S. Pat. No. 8,217,149, and US 2013/034559.

Other examples of mAbs that bind to human PD-L1, and useful in the treatment method, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MEDI4736, MSB0010718C.

As used herein, a “PD-L1 binding antagonist” is a molecule that decreases, blocks, inhibits, abrogates or interferes with signal transduction resulting from the interaction of PD-L1 with either one or more of its binding partners, such as PD-1 and/or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, PD-L1 binding antagonists include anti-PD-L1 antibodies and antigen-binding fragments thereof, immunoadhesins, fusion proteins, oligopeptides, small molecule antagonist, polynucleotide antagonists, and other molecules that decrease, block, inhibit, abrogate or interfere with signal transduction resulting from the interaction of PD-L1 with one or more of its binding partners, such as PD-1 and/or B7-1. In one embodiment, a PD-L1 binding antagonist reduces the negative signal mediated by or through cell surface proteins expressed on T lymphocytes, and other cells, mediated signaling through PD-L1 or PD-1 so as render a dysfunctional T-cell less dysfunctional. In some embodiments, a PD-L1 binding antagonist is an anti-PD-L1 antibody. In a specific aspect, an anti-PD-L1 antibody is YW243.55.570. In another specific aspect, an anti-PD-L1 antibody is MDX-1 105. In still another specific aspect, an anti-PD-L1 antibody is MPDL3280A (atezolizumab). In still another specific aspect, an anti-PD-L1 antibody is MEDI4736 (durvalumab). In still another specific aspect, an anti-PD-L1 antibody is MSB001 0718C (avelumab). MDX-1 105, also known as BMS-936559, is an anti-PD-L1 antibody described in WO2007/005874. Antibody YW243.55.S70 is an anti-PD-L1 antibody described in WO 2010/077634 and U.S. Pat. No. 8,217,149, the entirety of each of which is incorporated herein by reference.

Additional examples of other therapeutic agents (anti-neoplastic agent or immuno-modulators) for use in combination or co-administered with a RIP1 inhibitor compound are PD-1 antagonist.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H₁, B7-4, CD274 and B7-H for PD-L1; and PDCD1L2, PDL2, B7-DC, Btdc and CD273 for PD-L2. In any embodiments of the aspects or embodiments of the present invention in which a human individual is to be treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in any of the aspects of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the various aspects and embodiments of the present invention, are described in U.S. Pat. Nos. 7,488,802, 7,521,051, 8,008,449, 8,354,509, 8,168,757, WO2004/004771, WO2004/072286, WO2004/056875, US2011/0271358 and US2018/0030137.

Specific anti-human PD-1 mAbs useful as the PD-1 antagonist in any of the aspects and embodiments of the present invention include: MK-3475, a humanized IgG4 mAb with the structure described in WHO Drug Information, Vol. 27, No. 2, pages 161-162 (2013) and which comprises the heavy and light chain amino acid sequences shown in FIG. 6; nivolumab, a human IgG4 mAb with the structure described in WHO Drug Information, Vol. 27, No. 1, pages 68-69 (2013) and which comprises the heavy and light chain amino acid sequences shown in FIG. 7; the humanized antibodies h409A11, h409A16 and h409A17, which are described in WO2008/156712, and AMP-514, which is being developed by Medimmune.

Other PD-1 antagonists useful in the any of the aspects and embodiments of the present invention include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

KEYTRUDA/pembrolizumab is an anti-PD-1 antibody marketed for the treatment of lung cancer by Merck. The amino acid sequence of pembrolizumab and methods of using are disclosed in U.S. Pat. No. 8,168,757.

In one embodiment, any mouse or chimeric sequences of any anti-PD-1 of a combination of the invention, or a method or use thereof, are engineered to make a humanized antibody.

Opdivo/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of PI3K/Akt pathway activation. Other names for nivolumab include: BMS-936558, MDX-1106, and ONO-4538. The amino acid sequence for nivolumab and methods of using and making are disclosed in U.S. Pat. No. 8,008,449.

Additional examples of other therapeutically active agents (anti-neoplastic agent) for use in combination or co-administered with a compound of Formula (I), (II), or (III) are antibodies to ICOS, in particular, agonist antibodies of human ICOS.

ICOS is a co-stimulatory T cell receptor with structural and functional relation to the CD28/CTLA-4-Ig superfamily (Hutloff, et al., “ICOS is an inducible T-cell costimulator structurally and functionally related to CD28”, Nature, 397: 263-266 (1999)). Activation of ICOS occurs through binding by ICOS-L (B7RP-1/B7-H₂). Neither B7-1 nor B7-2 (ligands for CD28 and CTLA4) bind or activate ICOS. However, ICOS-L has been shown to bind weakly to both CD28 and CTLA-4 (Yao S et al., “B7-H₂ is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naïve T_{H}1 and T_{H}2 effector T cell populations (Paulos C M et al., “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu E, et al., “Convergent and divergent effects of costimulatory molecules in conventional and regulatory CD4+ T cells”, Proc Natal Acad Sci USA, 110(3); 1023-8 (2013)).

CDRs for murine antibodies to human ICOS having agonist activity are shown in PCT/EP2012/055735 (WO 2012/131004). Antibodies to ICOS are also disclosed in WO 2008/137915, WO 2010/056804, EP 1374902, EP1374901, and EP1125585.

Agonist antibodies to ICOS or ICOS binding proteins are disclosed in WO2012/13004, WO2014/033327, WO2016/120789, US20160215059, and US20160304610. Exemplary antibodies in US2016/0304610 include 37A10S71. Sequences of 37A10S713 are reproduced below as SEQ ID Nos: 13-20.

(SEQ. ID NO: 13)
EVQLVESGG LVQPGGSLRL SCAASGFTFS DYWMDWVRQA
PGKGLVWVSN IDEDGSITEY SPFVKGRFTI SRDNAKNTLY
LQMNSLRAED TAVYYCTRWG RFGFDSWGQG TLVTVSS
(SEQ. ID NO: 14)
DIVMTQSPDS LAVSLGERAT INCKSSQSLL SGSFNYLTWY
QQKPGQPPKL LIFYASTRHT GVPDRFSGSG SGTDFTLTIS
SLQAEDVAVY YCHHHYNAPP TFGPGTKVDI K
(SEQ. ID NO: 15)
GFTFSDYWMD
(SEQ. ID NO: 16)
NIDEDGSITEYSPFVKG
(SEQ. ID. NO: 17)
WGRFGFDS
(SEQ. ID NO: 18)
KSSQSLLSGSFNYLT
(SEQ. ID NO: 19)
YASTRHT
(SEQ. ID NO: 20)
HHHYNAPPT

In one embodiment, the immuno-modulator is an agonist antibody to human ICOS. In one embodiment, agonist antibodies to ICOS include ICOS binding proteins or antigen binding portions thereof comprising one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR as disclosed in WO2016/120789, which is incorporated by reference in its entirety herein. In one embodiment, the ICOS binding protein or antigen binding portion thereof is an agonist antibody to ICOS comprising a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 as set forth in WO2016/120789 wherein said ICOS binding protein specifically binds to human ICOS. In one embodiment, the ICOS binding protein is an agonist antibody to ICOS comprising a V_{H} domain comprising the amino acid sequence set forth in SEQ ID NO:7 and a V_{L} domain comprising the amino acid sequence set forth in SEQ ID NO:8 as set forth in WO2016/120789. SEQ ID NOs:1-8 as set forth in WO2016/120789 are provided below and in the sequence listing.

Accordingly, ICOS binding proteins are provided, which comprises any one or a combination of the following CDRs:

CDRH1:
(SEQ ID NO: 1)
DYAMH
CDRH2:
(SEQ ID NO: 2)
LISIYSDHTNYNQKFQG
CDRH3:
(SEQ ID NO: 3)
NNYGNYGWYFDV
CDRL1:
(SEQ ID NO: 4)
SASSSVSYMH
CDRL2:
(SEQ ID NO: 5)
DTSKLAS
CDRL3:
(SEQ ID NO: 6)
FQGSGYPYT

In one embodiment of the present invention the ICOS binding protein comprises CDRH1 (SEQ ID NO:1), CDRH2 (SEQ ID NO:2), and CDRH3 (SEQ ID NO:3) in the heavy chain variable region having the amino acid sequence set forth in SEQ ID NO:7. ICOS binding proteins of the present invention comprising the humanized heavy chain variable region set forth in SEQ ID NO:7 are designated as “H₂.” In some embodiments, the ICOS binding proteins of the present invention comprise a heavy chain variable region having at least 90% sequence identity to SEQ ID NO:7. Suitably, the ICOS binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:7.

Humanized Heavy Chain (V_{H}) Variable Region (H₂):

(SEQ ID NO: 7)
QVQLVQSGAE VKKPGSSVKV SCKASGYTFT DYAMHWVRQA
PGQGLEWMGL ISIYSDHTNY NQKFQGRVTI TADKSTSTAY
MELSSLRSED TAVYYCGRNN YGNYGWYFDV WGQGTTVTVS S

In one embodiment of the present invention the ICOS binding protein comprises CDRL1 (SEQ ID NO:4), CDRL2 (SEQ ID NO:5), and CDRL3 (SEQ ID NO:6) in the light chain variable region having the amino acid sequence set forth in SEQ ID NO:8. ICOS binding proteins of the present invention comprising the humanized light chain variable region set forth in SEQ ID NO:8 are designated as “L5.” Thus, an ICOS binding protein of the present invention comprising the heavy chain variable region of SEQ ID NO:7 and the light chain variable region of SEQ ID NO:8 can be designated as H₂L5 herein.

In some embodiments, the ICOS binding proteins of the present invention comprise a light chain variable region having at least 90% sequence identity to the amino acid sequence set forth in SEQ ID NO:8. Suitably, the ICOS binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:8.

### Humanized Light Chain (V_{L}) Variable Region (L5)

(SEQ ID NO: 8)
EIVLTQSPAT LSLSPGERAT LSCSASSSVS YMHWYQQKPG
QAPRLLIYDT SKLASGIPAR FSGSGSGTDY TLTISSLEPE
DFAVYYCFQG SGYPYTFGQG TKLEIK

Human IgG1 constant regions containing specific mutations or altered glycosylation on residue Asn297 have also been described to enhance binding to Fc receptors. In some cases, these mutations have also been shown to enhance ADCC and CDC, see for example, Kellner (2013).

In one embodiment of the present invention, such mutations are in one or more of positions selected from 239, 332 and 330 (IgG1), or the equivalent positions in other IgG isotypes. Examples of suitable mutations are S239D and I332E and A330L. In one embodiment, the antigen binding protein of the invention herein described is mutated at positions 239 and 332, for example S239D and I332E or in a further embodiment it is mutated at three or more positions selected from 239 and 332 and 330, for example S239D and I332E and A330L (EU index numbering).

In one embodiment, the ICOS binding proteins comprise a scaffold selected from human IgG1 isotype or variant thereof and human IgG4 isotype or variant thereof. Suitably, the scaffold comprises a human IgG4 isotype scaffold or variant thereof. In one aspect, the scaffold comprises a hIgG4PE scaffold.

In one embodiment, the ICOS binding protein is a monoclonal antibody. Suitably the ICOS binding protein is a humanized monoclonal antibody. In one aspect, the monoclonal antibodies of the present invention can be fully human.

In another aspect, the ICOS binding protein is a fragment which is a Fab, Fab′, F(ab′)₂, Fv, diabody, triabody, tetrabody, miniantibody, minibody, isolated V_{H} or isolated V_{L}. In one embodiment, the ICOS binding protein is an antigen binding portion thereof.

In some aspects, the ICOS binding protein binds to human ICOS with an affinity of stronger than 0.6 nM. In one aspect, the affinity is 100 nM or stronger. In one embodiment, the ICOS binding protein has a KD of 100 nM for ICOS. Suitably, the KD of the ICOS binding protein for ICOS is 100 nM or less, 50 nM or less, 25 nM or less, 10 nM or less, 2 nM or less or 1 nM or less.

By “an anti-CTLA4 antibody” is meant an antibody that selectively binds a CTLA4 polypeptide. Exemplary anti-CTLA4 antibodies are described for example at U.S. Pat. Nos. 6,682,736; 7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA4 antibody.

YERVOY (ipilimumab) is a fully human CTLA-4 antibody marketed by Bristol Myers Squibb. The protein structure of ipilimumab and methods are using are described in U.S. Pat. Nos. 6,984,720 and 7,605,238.

In one embodiment, any mouse or chimeric sequences of any anti-CTLA-4 antigen binding protein of a combination of the invention, or a method or use thereof, are engineered to make a humanized antibody.

CD134, also known as OX40, is a member of the TNFR-superfamily of receptors which is not constitutively expressed on resting naïve T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX-40 antibodies, OX-40 fusion proteins and methods of using them are disclosed in U.S. Pat. Nos. 7,504,101; 7,758,852; 7,858,765; 7,550,140; 7,960,515; WO2012027328; WO2013028231.

Antigen binding proteins that bind human OX40 (also referred to as OX40 receptor) are provided herein (i.e., an anti-OX40 antigen binding protein and an anti-human OX40 receptor (hOX-40R) antigen binding protein, sometimes referred to herein as an “anti-OX40 ABP”, such as an “anti-OX40 antibody”). These antigen binding proteins, such as antibodies, are useful in the treatment or prevention of acute or chronic diseases or conditions whose pathology involves OX40 signaling. In one aspect, an antigen binding protein, or isolated human antibody or functional fragment of such protein or antibody, that binds to human OX40R and is effective as a cancer treatment or treatment against disease is described, for example in combination with another compound such as an anti-PD-1 antigen binding protein, suitably an antagonist anti-PD-1 antigen binding protein. Any of the antigen binding proteins or antibodies disclosed herein may be used as a medicament. Any one or more of the antigen binding proteins or antibodies may be used in the methods or compositions to treat cancer, e.g., those disclosed herein. The anti-OX40 ABPs are agonist antibodies, e.g., agonists of OX40 (i.e., of OX40 receptor).

In one embodiment, the OX40 antigen binding protein is one disclosed in WO2012/027328 (PCT/US2011/048752), international filing date 23 Aug. 2011. In another embodiment, the antigen binding protein comprises the CDRs of an antibody disclosed in WO2012/027328 (PCT/US2011/048752), international filing date 23 Aug. 2011, or CDRs with 90% identity to the disclosed CDR sequences. In a further embodiment, the OX-40 antigen binding protein comprises a VH, a VL, or both of an antibody disclosed in WO2012/027328 (PCT/US2011/048752), international filing date 23 Aug. 2011, or a VH or a VL with 90% identity to the disclosed VH or VL sequences.

In another embodiment, the OX40 antigen binding protein is disclosed in WO2013/028231 (PCT/US2012/024570), international filing date 9 Feb. 2012, which is incorporated by reference in its entirety herein. In another embodiment, the antigen binding protein comprises the CDRs of an antibody disclosed in WO2013/028231 (PCT/US2012/024570), international filing date 9 Feb. 2012, or CDRs with 90% identity to the disclosed CDR sequences. In a further embodiment, the antigen binding protein comprises a VH, a VL, or both of an antibody disclosed in WO2013/028231 (PCT/US2012/024570), international filing date 9 Feb. 2012, or a VH or a VL with 90% identity to the disclosed VH or VL sequences. In one embodiment, the OX40 antigen binding protein is an isolated agonist antibody to OX40 comprising a light chain variable region having a sequence at least 90% identical to the amino acid sequence of SEQ ID NO:11 (set forth as SEQ ID NO:10 in WO2013/028231) and a heavy chain variable region having a sequence at least 90% identical to the amino acid sequence of SEQ ID NO:9 (set forth as SEQ ID NO:4 in WO2013/028231). In one embodiment, the OX40 antigen binding protein is an isolated antibody comprising a light chain variable comprising the amino acid sequence of SEQ ID NO:12 (set forth as SEQ ID NO:11 in WO2013/028231) and a heavy chain variable region comprising the amino acid sequence of SEQ ID NO:10 (set forth as SEQ ID NO:5 in WO2013/028231).

In one embodiment, the OX-40 antibody is an agonist antibody. In one embodiment the OX-40 or antigen binding portion thereof comprises a VH region having an amino acid sequence chosen from: an amino acid sequence at least 90% identical to the amino acid sequence as set forth in SEQ ID NO:9; an amino acid sequence at least 90% identical to the amino acid sequence as set forth in SEQ ID NO:10, and VL having an amino acid sequence chosen from: an amino acid sequence at least 90% identical to the amino acid sequence as set forth in SEQ ID NO:11; and an amino acid sequence at least 90% identical to the amino acid sequence as set forth in SEQ ID NO:12. Suitably, the OX-40 antibody of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:9. Suitably, the OX-40 antibody of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:10. Suitably, the OX-40 antibody of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:11. Suitably, the OX-40 antibody of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:12.

SEQ ID Nos: 4, 5, 10, and 11 as set forth in WO2013/028231 are presented below as SEQ ID Nos: 9-12.

(SEQ ID NO: 9)
Gln Ile Gln Leu Val Gln Ser Gly Pro Glu Leu Lys
Lys Pro Gly Glu Thr Val Lys Ile Ser Cys Lys Ala
Ser Gly Tyr Thr Phe Thr Asp Tyr Ser Met His Trp
Val Lys Gln Ala Pro Gly Lys Gly Leu Lys Trp Met
Gly Trp Ile Asn Thr Glu Thr Gly Glu Pro Thr Tyr
Ala Asp Asp Phe Lys Gly Arg Phe Ala Phe Ser Leu
Glu Thr Ser Ala Ser Thr Ala Tyr Leu Gln Ile Asn
Asn Leu Lys Asn Glu Asp Thr Ala Thr Tyr Phe Cys
Ala Asn Pro Tyr Tyr Asp Tyr Val Ser Tyr Tyr Ala
Met Asp Tyr Trp Gly His Gly Thr Ser Val Thr Val
Ser Ser
(SEQ ID NO: 10)
Gln Val Gln Leu Val Gln Ser Gly Ser Glu Leu Lys
Lys Pro Gly Ala Ser Val Lys Val Ser Cys Lys Ala
Ser Gly Tyr Thr Phe Thr Asp Tyr Ser Met His Trp
Val Arg Gln Ala Pro Gly Gln Gly Leu Lys Trp Met
Gly Trp Ile Asn Thr Glu Thr Gly Glu Pro Thr Tyr
Ala Asp Asp Phe Lys Gly Arg Phe Val Phe Ser Leu
Asp Thr Ser Val Ser Thr Ala Tyr Leu Gln Ile Ser
Ser Leu Lys Ala Glu Asp Thr Ala Val Tyr Tyr Cys
Ala Asn Pro Tyr Tyr Asp Tyr Val Ser Tyr Tyr Ala
Met Asp Tyr Trp Gly Gln Gly Thr Thr Val Thr Val
Ser Ser
(SEQ ID NO: 11)
Asp Ile Val Met Thr Gln Ser His Lys Phe Met Ser
Thr Ser Val Arg Asp Arg Val Ser Ile Thr Cys Lys
Ala Ser Gln Asp Val Ser Thr Ala Val Ala Trp Tyr
Gln Gln Lys Pro Gly Gln Ser Pro Lys Leu Leu Ile
Tyr Ser Ala Ser Tyr Leu Tyr Thr Gly Val Pro Asp
Arg Phe Thr Gly Ser Gly Ser Gly Thr Asp Phe Thr
Phe Thr Ile Ser Ser Val Gln Ala Glu Asp Leu Ala
Val Tyr Tyr Cys Gln Gln His Tyr Ser Thr Pro Arg
Thr Phe Gly Gly Gly Thr Lys Leu Glu Ile Lys
(SEQ ID NO: 12)
Asp Ile Gln Met Thr Gln Ser Pro Ser Ser Leu Ser
Ala Ser Val Gly Asp Arg Val Thr Ile Thr Cys Lys
Ala Ser Gln Asp Val Ser Thr Ala Val Ala Trp Tyr
Gln Gln Lys Pro Gly Lys Ala Pro Lys Leu Leu Ile
Tyr Ser Ala Ser Tyr Leu Tyr Thr Gly Val Pro Ser
Arg Phe Ser Gly Ser Gly Ser Gly Thr Asp Phe Thr
Phe Thr Ile Ser Ser Leu Gln Pro Glu Asp Ile Ala
Thr Tyr Tyr Cys Gln Gln His Tyr Ser Thr Pro Arg
Thr Phe Gly Gln Gly Thr Lys Leu Glu Ile Lys

Thus, in one embodiment methods of treating a human in need thereof are provided comprising administering a compound of Formula (I), (II), or (III) or a salt thereof and at least one immuno-modulator. In one embodiment, the immuno-modulator is selected from an ICOS antibody, an OX-40 antibody, a PD-L1 antibody, a CTLA4 antibody or a PD-1 antibody. In one embodiment, the human has cancer. Also provided herein is the use of a compound of Formula (I), (II), or (III), or a salt thereof in combination with at least one immuno-modulator for the treatment of a human in need thereof.

Described herein are combinations of a RIP1 inhibitor including compounds of Formulas (I), (II), or (III) and at least one immuno-modulator. Thus, as used herein the term “combination of the invention” or “combinations” refers to a combination comprising a compound of Formula I and at least one immuno-modulator each of which may be administered separately or simultaneously as described herein.

In one embodiment, a combination is provided comprising a RIP1 inhibitor compound and at least one other therapeutically active agent, wherein the at least one other therapeutically active agent is an immuno-modulator. In one embodiment, the RIP1 inhibitor compound is a compound of Formula I. In one embodiment, the RIP1 inhibitor compound is (S)-5-benzyl-N-(7,9-difluoro-2-oxo-2,3,4,5-tetrahydro-1H-benzo[b]azepin-3-yl)-4H-1,2,4-triazole-3-carboxamide. In one embodiment, the least one immuno-modulator comprises at least one anti-CTLA4, anti-PD-1, anti-PD-L1, anti-OX-40 antibody and/or anti-ICOS antibody.

In one embodiment, the immuno-modulator is selected from ipilimumab; tremelumumab; nivolumab; pembrolizumab; atezolizumab; durvalumumab; avelumab; at least one agonist antibody to human ICOS and/or at least one agonist antibody to human OX-40. In one embodiment, the combination comprises a compound of Formula I and an anti-PD-1 antibody selected from nivolumab and pembrolizumab.

In one embodiment, the combination comprises a RIP1 kinase inhibitor and an anti-ICOS antibody wherein the anti-ICOS antibody is an agonist antibody and wherein the anti-ICOS antibody comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the combination comprises a RIP1 kinase inhibitor and an anti-ICOS antibody wherein the anti-ICOS antibody is an agonist antibody and wherein the anti-ICOS antibody comprises a V_{H} domain comprising an amino acid sequence at least 90% identical to the amino acid sequence set forth in SEQ ID NO:13 and/or a V_{L} domain comprising an amino acid sequence at least 90% identical to the amino acid sequence as set forth in SEQ ID NO:14 wherein said ICOS binding protein specifically binds to human ICOS. In one embodiment, the ICOS antibody comprising the CDRs set forth in SEQ ID NOs:15-20.

In one embodiment, the combination comprises an ICOS antibody that binds to human ICOS with
(i) an association rate constant (km) of at least 1×10⁻¹ s⁻¹; and a dissociation rate constant (k_{off}) of less than 6×10⁻⁵ s⁻¹; or
(ii) a dissociation constant (K_{D}) of less than about 100 nM,
wherein the affinity is measured by BIAcore.

A combination kit comprising a combination according to any of the preceding claims together with one or more pharmaceutically acceptable carriers.

Also provided are pharmaceutical compositions comprising any of the combinations described herein together with a pharmaceutically acceptable diluent or carrier. In one embodiment, pharmaceutical compositions are provided comprising a therapeutically effective amount of a compound of Formula I and a second pharmaceutical composition comprising a therapeutically effective amount of an immuno-modulator.

In one embodiment, use of any combination or pharmaceutical composition of the present invention are provided for the treatment of cancer. In one embodiment, use of any combination or pharmaceutical composition of the present invention are provided in the manufacture of a medicament for the treatment of cancer.

In one embodiment, method of treating cancer in a human in need thereof are provided comprising administering a therapeutically effective amount of any combination or pharmaceutical composition of the invention. In one embodiment, the RIP1 inhibitor compound and the immuno-modulator are administered at the same time. In one embodiment, the RIP1 inhibitor and the immuno-modulator are administered sequentially, in any order. In one embodiment, the RIP1 inhibitor is administered orally. In one embodiment, at least one immuno-modulator is administered systemically, e.g. intravenously.

In one embodiment, the cancer is a solid tumor. In one embodiment, the cancer is selected from the group consisting of: pancreatic cancer, metastatic adenocarcinoma of the pancreas, pancreatic ductal adenocarcinoma, a malignancy of the endocrine cells in the pancreas, hepatocellular carcinoma, mesothelioma, melanoma, colorectal cancer, acute myeloid leukemia, metastasis, glioblastoma, breast cancer, gallbladder cancer, clear cell renal carcinoma, non-small cell lung carcinoma, and radiation induced necrosis. In one embodiment, the cancer is Pancreatic ductal adenocarcinoma (PDA).

In one embodiment, the administration of said combination or pharmaceutical compositions of the present invention statistically significantly reduces the tumor size of at least one solid tumor in said human compared to said RIP1 kinase inhibitor and said immuno-modulator administered as monotherapy.

A compound having the formula:

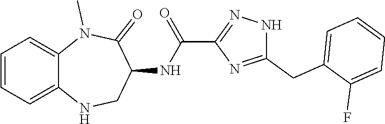 [SMILES: CN1C(=O)[C@@H](NC(=O)c2n[nH]c(Cc3ccccc3F)n2)CNc2ccccc21]

or a salt thereof, or a tautomer thereof.

In one embodiment, methods of treating cancer are provided wherein the combination comprises:

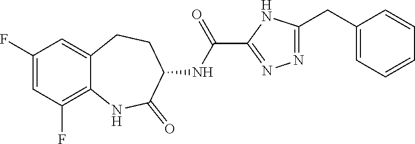 [SMILES: O=C(N[C@H]1CCc2cc(F)cc(F)c2NC1=O)c1nnc(Cc2ccccc2)[nH]1]

or a tautomer thereof; or a pharmaceutically acceptable salt thereof
wherein the cancer is selected from pancreatic cancer, metastatic adenocarcinoma of the pancreas, pancreatic ductal adenocarcinoma, and a malignancy of the endocrine cells in the pancreas, and
wherein the at least one immuno-modulator comprises at least one anti-CTLA4, anti-PD-1, anti-PD-L1, anti-OX-40 antibody and/or anti-ICOS antibody.

As used herein, the terms “cancer,” “neoplasm,” and “tumor,” are used interchangeably and in either the singular or plural form, refer to cells that have undergone a malignant transformation or undergone cellular changes that result in aberrant or unregulated growth or hyperproliferation Such changes or malignant transformations usually make such cells pathological to the host organism, thus precancers or precancerous cells that are or could become pathological and require or could benefit from intervention are also intended to be included. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumors of any stage, including what a clinician refers to as precancer, tumors, in situ growths, as well as late stage metastatic growths. Tumors may be hematopoietic tumor, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

The invention further provides pharmaceutical compositions, which include one or more of the components herein, and one or more pharmaceutically acceptable carriers, diluents, or excipients. The combination of the invention may comprise two pharmaceutical compositions, one comprising a compound of Formula I, and the other comprising an immuno-modulator, each of which may have the same or different carriers, diluents or excipients. The carrier(s), diluent(s) or excipient(s) must be acceptable in the sense of being compatible with the other ingredients of the formulation, capable of pharmaceutical formulation, and not deleterious to the recipient thereof.

The components of the combination of the invention, and pharmaceutical compositions comprising such components may be administered in any order, and in different routes; the components and pharmaceutical compositions comprising the same may be administered simultaneously.

In accordance with another aspect of the invention there is also provided a process for the preparation of a pharmaceutical composition including admixing a component of the combination of the invention and one or more pharmaceutically acceptable carriers, diluents or excipients.

A compound that inhibits RIP1 kinase, particularly a compound of Formula (I), (II), or (III), or a pharmaceutically acceptable salt thereof, may be administered in combination with other anti-inflammatory agents for any of the indications above, including oral or topical corticosteroids (such as prednisone (Deltasone®) and bundesonide), anti-TNF agents (including anti-TNF biologic agents), 5-aminosalicyclic acid and mesalamine preparations, hydroxycloroquine, thiopurines (azathioprin, mercaptopurin), methotrexate, cyclophosphamide, cyclosporine, calcineurin inhibitors (cyclosporine, pimecrolimus, tacrolimus), mycophenolic acid (CellCept®), mTOR inhibitors (temsirolimus, everolimus), JAK inhibitors (tofacitinib), (Xeljan®)), Syk inhibitors (fostamatinib), anti-IL6 biologics, anti-IL1 (anakinra (Kineret®), canakinumab (Ilaris®), rilonacept (Arcalyst®)), anti-IL12 and IL23 biologics (ustekinumab (Stelara®)), anti-IL17 biologics (secukinumab), anti-CD22 (epratuzumab), anti-integrin agents (natalizumab (Tysabri®)), vedolizumab (Entyvio®)), anti-IFNa (sifalimumab), anti-CD20 or CD4 biologics and other cytokine inhibitors or biologics to T-cell or B-cell receptors or interleukins.

Examples of other suitable anti-inflammatory biologic agents include Actemra® (anti-IL6R mAb), anti-CD20 mAbs (rituximab (Rituxan®) and ofatumumab (Arzerra®)), abatacept (Orencia®), anakinra (Kineret®), ustekinumab (Stelara®), and belimumab (Benlysta®). Examples of other suitable anti-inflammatory biologic agents include Actemra® (tocilizumab, anti-IL6R mAb), anti-CD20 mAbs (rituximab (Rituxan®) and ofatumumab (Arzerra®)), abatacept (Orencia®), anakinra (Kineret®), Canakinumab (Ilaris®), rilonacept (Arcalyst®), secukinumab, epratuzumab, sifalimumab, ustekinumab (Stelara®), and belimumab (Benlysta®). Examples of suitable anti-TNF biologic agents include etanecerpt (Enbrel®), adalimumab (Humira®), infliximab (Remicade®), certolizumab (Cimzia®), and golimumab (Simponi®).

In the treatment of pancreatic cancer (particularly metastatic adenocarcinoma of the pancreas, pancreatic ductal adenocarcinoma and/or malignancies of the endocrine cells in the pancreas), a compound that inhibits RIP1 kinase, particularly a compound of Formula (I), (II), or (III), or a pharmaceutically acceptable salt thereof, may be administered in combination with gemcitabine, FOLFIRINOX regimen (folinic acid (leucovorin), fluorouracil, irinotecan (Camptosar®), oxaliplatin (Eloxatin®), nab-paclitaxel (protein-bound paclitaxel, or nanoparticle albumin-bound paclitaxel), and immunotherapeutic agents (particularly an immuno-modulator or immunomodulatory agent including a checkpoint inhibitor antibody, for example antibody to PD-1, PD-L1, OX40, ICOS, CTLA4). In one embodiment, methods are provided for treating pancreatic cancer comprising administering to a human in need thereof a therapeutically effective amount of a compound of Formula (I), (II), or (III) or a pharmaceutically acceptable salt thereof and a PD-1 antibody. In one aspect, the PD-1 antibody is pembrolizumab or nivolumumab. In one embodiment, methods are provided for treating pancreatic cancer comprising administering to a human in need thereof a therapeutically effective amount of a compound of Formula (I), (II), or (III) or a pharmaceutically acceptable salt thereof and an ICOS binding protein or antigen binding portion thereof. In one embodiment, the ICOS binding protein or antigen binding portion thereof is an agonist antibody to ICOS comprising a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 as set forth in WO2016/120789 wherein said ICOS binding protein specifically binds to human ICOS.

In the treatment of hepatocellular carcinoma, a compound that inhibits RIP1 kinase, particularly a compound of Formula (I), (II), or (III), or a pharmaceutically acceptable salt thereof, may be administered in combination with sorafenib, gemcitabine, oxaliplatin, capecitabine, doxorubicin, and immunotherapeutic agents (antibodies to PD-1, PD-L1, OX40, ICOS, CTLA4) and as an adjuvant to liver transplant.

In the treatment of melanoma, a compound that inhibits RIP1 kinase, particularly a compound of Formula (I), (II), or (III), or a pharmaceutically acceptable salt thereof, may be administered in combination with immunotherapeutic agents (antibodies to PD-1, PD-L1, OX40, ICOS, CTLA4).

In the treatment of colorectal cancer, a compound that inhibits RIP1 kinase, particularly a compound of Formula (I), (II), or (III), or a pharmaceutically acceptable salt thereof, may be administered in combination with immunotherapeutic agents (antibodies to PD-1, PD-L1, OX40, ICOS, CTLA4).

In the treatment of acute myeloid leukemia, a compound that inhibits RIP1 kinase, particularly a compound of Formula (I), (II), or (III), or a pharmaceutically acceptable salt thereof, may be administered as an adjuvant to ALLO transplants.

In the treatment of glioblastoma, a compound that inhibits RIP1 kinase, particularly a compound of Formula (I), (II), or (III), or a pharmaceutically acceptable salt thereof, may be administered in combination with temozolomide, procarbazine, nitrosourea, and as an adjuvant to radiation.

In the treatment of breast cancer, a compound that inhibits RIP1 kinase, particularly a compound of Formula (I), (II), or (III), or a pharmaceutically acceptable salt thereof, may be administered in combination with PARP inhibitors, anti-her2 therapies, TDM-1, SERD, nab-paclitaxel (protein-bound paclitaxel, or nanoparticle albumin-bound paclitaxel), and immunotherapeutic agents (antibodies to PD-1, PD-L1, OX40, ICOS, CTLA4).

In the treatment of gallbladder cancer, a compound that inhibits RIP1 kinase, particularly a compound of Formula (I), (II), or (III), or a pharmaceutically acceptable salt thereof, may be administered in combination with chemotherapy and radiation therapy.

In the treatment of clear cell renal carcinoma (cc-RCC), a compound that inhibits RIP1 kinase, particularly a compound of Formula (I), (II), or (III), or a pharmaceutically acceptable salt thereof, may be administered in combination with VEGF inhibitors, Tyrosine kinase inhibitors, and/or immunotherapeutic agents (antibodies to PD-1, PD-L1, OX40, ICOS, CTLA4).

In the treatment of non-small cell lung carcinoma (NSCLC), a compound that inhibits RIP1 kinase, particularly a compound of Formula (I), (II), or (III), or a pharmaceutically acceptable salt thereof, may be administered in combination with immunotherapeutic agents (antibodies to PD-1, PD-L1, OX40, ICOS, CTLA4).

The pharmaceutical compositions of the invention typically contain one compound useful in this invention. However, in certain embodiments, the pharmaceutical compositions of the invention contain more than one compound useful in this invention. In other embodiments, the pharmaceutical compositions of the invention may comprise one or more additional therapeutic agents, specifically one or two other therapeutically active agents, more specifically one other therapeutically active agent.

The RIP1 inhibitor compound, specifically, the compound(s) useful in the invention, particularly the compounds of Formula (I), (II), or (III), or pharmaceutically acceptable salts thereof, and the other therapeutic agent(s) may be administered together in a single pharmaceutical composition or separately and, when administered separately this may occur simultaneously or sequentially in any order. The amounts of the compound(s) of the invention, particularly a compound of Formula (I), (II), or (III), or pharmaceutically acceptable salts thereof, and the other therapeutic agent(s) and the relative timings of administration will be selected in order to achieve the desired combined therapeutic effect.

Thus, in a further aspect, there is provided a combination comprising a RIP1 inhibitor, particularly a compound of Formula (I), (II), or (III), or a pharmaceutically acceptable salt thereof, together with one or more other therapeutic agents, specifically one or two other therapeutically active agents, more specifically one other therapeutically active agent. In one aspect, there is provided a combination comprising (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, or a pharmaceutically acceptable salt thereof, together with one or more other therapeutic agents, specifically one or two other therapeutically active agents, more specifically one other therapeutically active agent.

Thus, in one aspect of this invention, a RIP1 inhibitor compound, particularly a compound of Formula (I), (II), or (III), or a pharmaceutically acceptable salt thereof, or a pharmaceutical composition comprising a RIP1 inhibitor compound, particularly a compound of Formula (I), (II), or (III), or a pharmaceutically acceptable salt thereof, may be used in combination with or include one or more other therapeutic agents.

For example, amelioration of tissue damage may be achieved by treatment with a compound of Formula (I), (II), or (III), or a pharmaceutically acceptable salt thereof, and at least one other therapeutically active agent during transplant surgery. Amelioration of tissue damage may also be achieved by short-term treatment of a patient with a compound of Formula (I), (II), or (III), or a pharmaceutically acceptable salt thereof, and at least one other therapeutic ally active agent after transplant surgery. Amelioration of tissue damage ex vivo, that is ex vivo preservation of tissues, organs and cells may also be achieved by short-term treatment of tissues, organs and cells with a compound of Formula (I), (II), or (III), or a pharmaceutically acceptable salt thereof, and at least one other therapeutic ally active agent, prior to or during transplant surgery.

Treatment of RIP1-mediated disease conditions, or more broadly, treatment of diseases where increased intestinal permeability is implicated in the pathogenesis, may be achieved using a RIP1 inhibitor compound as a monotherapy, or in dual or multiple combination therapy, such as in combination with other agents or treatments which may enhance gut recovery or attenuate bacterial translocation of the systemic circulation. In one embodiment of this invention, compounds useful in this invention, particularly a compound of Formula (I), (II), or (III), or a pharmaceutically acceptable salt thereof, may be administered in combination with at least one other therapeutically active agent selected from selective gut decontamination (may include a combination of oral non-absorbable antibiotics (Rifaximin, Paromycin, Vancomycin, Neomycin, Metronidazole) and a brief course of systemic antibiotics predominantly effective against gram negative organisms), broad-spectrum antibiotics, proton pump inhibitors (e.g. omeprazole, lansoprazole, pantoprazole, esomeprazole), steroids (e.g. prednisolone, methylprednisolone, hydrocortisone, oxandrolone, dexamethasone), GI motility agents (metoclopramide, erythromycin, azithromycin, domperidone, cisapride, nortryptilline, amitryptilline, camicinal, relamorelin), laxatives (e.g. senna, lactulose, polyethylene glycol), vasopressors (including vasopressors administered during the treatment of hypovolaemic shock e.g. Dopamine, Dobutamine, Norepinephrine, Dopexamine), total parenteral nutrition, enteral nutrition, probiotics (preparations of, for example, lactobacilli, bifidobacteria), supplemental Glutamine or Arginine administration, fish oils, propranolol, anti-coagulant therapy with unfractionated or low molecular weight heparins, IVIg, Cyclosporine, and anti-TNF therapies (Infliximab, etanercept).

In another embodiment, compounds useful in this invention, particularly a compound of Formula (I), (II), or (III), or a pharmaceutically acceptable salt thereof, may be administered in combination with other anti-inflammatory agents for any of the indications herein, including oral corticosteroids (such as prednisone, methylprednisolone, Deltasone®, and bundesonide), anti-TNF agents (including anti-TNF biologic agents), 5-aminosalicyclic acid and mesalamine preparations, hydroxycloroquine, thiopurines (azathioprin, mercaptopurin), methotrexate, cyclophosphamide, cyclosporine, JAK inhibitors (tofacitinib), anti-IL6 biologics, anti-IL1 or IL12 or IL23 biologics (ustekinumab (Stelara®)), anti-integrin agents (natalizumab (Tysabri®)), anti-CD20 or CD4 biologics and other cytokine inhibitors or biologics to T-cell or B-cell receptors or interleukins, calcineurin inhibitors (cyclosporine, pimecrolimus, tacrolimus), mycophenolic acid (CellCept®), and mTOR inhibitors (temsirolimus, everolimus).

The invention is further directed to a pharmaceutical composition comprising a compound of Formula (I), (II), or (III), or a pharmaceutically acceptable salt thereof, and at least one pharmaceutically acceptable excipient and at least one other therapeutically active agent, specifically one or two other therapeutically active agents, more specifically one other therapeutically active agent. In one embodiment, there is provided a pharmaceutical composition comprising ((S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, or a pharmaceutically salt thereof, at least one pharmaceutically acceptable excipient, and at least one other therapeutically active agent, specifically one or two other therapeutically active agents, more specifically one other therapeutically active agent. In another embodiment, there is provided a pharmaceutical composition comprising (S)-5-(2-fluorobenzyl)-N-(1-methyl-2-oxo-2,3,4,5-tetrahydro-1H-benzo[b][1,4]diazepin-3-yl)-1H-1,2,4-triazole-3-carboxamide, or a tautomer thereof, at least one pharmaceutically acceptable excipient, and at least one other therapeutically active agent, specifically one or two other therapeutically active agents, more specifically one other therapeutically active agent.

Accordingly, in one embodiment, this invention provides a method of treating cancer in a human in need thereof comprising administering to the human a combination or pharmaceutical composition comprising a RIP1 inhibitor compound and at least one immuno-modulator.

In another embodiment, this invention provides a method of treating cancer in a human in need thereof comprising administering to the human a combination or pharmaceutical composition comprising a RIP1 inhibitor compound and at least one immuno-modulator,
wherein the cancer is selected from pancreatic cancer, metastatic adenocarcinoma of the pancreas, pancreatic ductal adenocarcinoma, and a malignancy of the endocrine cells in the pancreas, and
wherein the at least one immuno-modulator comprises at least one anti-CTLA4, anti-PD-1, anti-PD-L1, anti-OX-40 antibody and/or anti-ICOS antibody.

In one embodiment, a combination or pharmaceutical composition of this invention comprises:

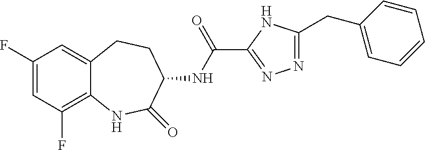 [SMILES: O=C(N[C@H]1CCc2cc(F)cc(F)c2NC1=O)c1nnc(Cc2ccccc2)[nH]1]

or a tautomer thereof; or a pharmaceutically acceptable salt thereof
at least one anti-CTLA4, anti-PD-1, anti-PD-L1, anti-OX-40 antibody and/or anti-ICOS antibody.

The pharmaceutical compositions of the invention may be prepared and packaged in bulk form wherein an effective amount of a compound useful in this invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound useful in this invention (i.e., a compound of Formula (I), (II), or (III), or a salt, particularly a pharmaceutically acceptable salt, thereof). When prepared in unit dosage form, the pharmaceutical compositions may contain from 1 mg to 1000 mg of a compound useful in this invention.

As provided herein, unit dosage forms (pharmaceutical compositions) containing from 1 mg to 1000 mg of a compound useful in this invention may be administered one, two, three, or four times per day, preferably one, two, or three times per day, and more preferably, one or two times per day, to effect treatment of a RIP1 kinase-mediated disease or disorder.

As used herein, “pharmaceutically acceptable excipient” means a material, composition or vehicle involved in giving form or consistency to the composition. Each excipient must be compatible with the other ingredients of the pharmaceutical composition when commingled such that interactions which would substantially reduce the efficacy of the compound useful in this invention when administered to a patient and interactions which would result in pharmaceutical compositions that are not pharmaceutically acceptable are avoided. In addition, each excipient must of course be of sufficiently high purity to render it pharmaceutically acceptable.

The compounds useful in this invention and the pharmaceutically acceptable excipient or excipients will typically be formulated into a dosage form adapted for administration to the patient by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound or compounds useful in this invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company). Accordingly, another embodiment of this invention is a method of preparing a pharmaceutical composition comprising the step of admixing a compound of Formula (I), (II), or (III), or a pharmaceutically acceptable salt, thereof, with at least one pharmaceutically acceptable excipient.

In one aspect, the invention is directed to a solid oral dosage form such as a tablet or capsule comprising an effective amount of a compound useful in this invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In another aspect, the invention is directed to an injection or continuous infusion form (examples include, but are not limited to, intravenous, intraperitoneal, intradermal, subcutaneous, intramuscular and intraportal). In one embodiment, the composition is suitable for intravenous administration.

In another aspect, the invention is directed to a topical dosage form such as a cream, ointment, lotion, paste, or gel comprising an effective amount of a compound useful in this invention and at least one pharmaceutically acceptable excipient. Lipophilic formulations, such as anhydrous creams and ointments, generally will have a base derived from fatty alcohols, and polyethylene glycols. Additional additives include alcohols, non-ionic surfactants, and antioxidants. For ointments, the base normally will be an oil or mixture of oil and wax, e.g., petrolatum. Also, an antioxidant normally will be included in minor amounts. Because the compositions are applied topically and the effective dosage can be controlled by the total composition applied, the percentage of active ingredient in the composition can vary widely. Convenient concentrations range from 0.5% to 20%.

Topically applied gels can also be a foamable suspension gel comprising a compound useful in this invention, as an active agent, one or more thickening agents, and optionally, a dispersing/wetting agent, a pH-adjusting agent, a surfactant, a propellent, an antioxidant, an additional foaming agent, a chelating/sequestering agent, a solvent, a fragrance, a coloring agent, a preservative, wherein the gel is aqueous and forms a homogenous foam.

In one aspect, the invention is directed to a topical dosage form that can be administered by inhalation, that is, by intranasal and oral inhalation administration. Appropriate dosage forms for such administration, such as an aerosol formulation or a metered dose inhaler, may be prepared by conventional techniques. Intranasal sprays may be formulated with aqueous or non-aqueous vehicles with the addition of agents such as thickening agents, buffer salts or acid or alkali to adjust the pH, isotonicity adjusting agents or anti-oxidants. Solutions for inhalation by nebulization may be formulated with an aqueous vehicle with the addition of agents such as acid or alkali, buffer salts, isotonicity adjusting agents or antimicrobials.

Formulations for administration by inhalation or foamable gel often require the use of a suitable propellant. Capsules and cartridges of e.g. gelatin for use in an inhaler or insufflator may be formulated using a suitable powder base such as lactose or starch.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

The reactions described herein are applicable for producing compounds useful in this invention having a variety of different substituent groups (e.g., R¹, R², etc.), as defined herein. The skilled artisan will appreciate that if a particular substituent is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999).

Names for the intermediate and final compounds described herein were generated using the software naming program ACD/Name Pro V6.02 available from Advanced Chemistry Development, Inc., 110 Yonge Street, 14^{th} Floor, Toronto, Ontario, Canada, M5C 1T4 (http://www.acdlabs.com/) or the naming program in ChemDraw, Struct=Name Pro 12.0, as part of ChemBioDraw Ultra, available from CambridgeSoft. 100 CambridgePark Drive, Cambridge, Mass. 02140 USA (www.cambridgesoft.com).

It will be appreciated by those skilled in the art that in certain instances these programs may name a structurally depicted compound as a tautomer of that compound. It is to be understood that any reference to a named compound or a structurally depicted compound is intended to encompass all tautomers of such compounds and any mixtures of tautomers thereof.

In the following experimental descriptions, the following abbreviations may be used:

| Abbreviation | Meaning |
|---|---|
| 2-MeTHF | 2-methyltetrahydrofuran |
| Aliquat ® 336 | Trioctylmethylammonium chloride |
| ACN | acetonitrile |
| Ac₂O | acetic anhydride |
| AcOH | acetic acid |
| aq. | aqueous |
| BnOH | benzyl alcohol |
| BOC, tBOC, Boc | tert-butoxycarbonyl |
| Brine | saturated aqueous solution of sodium chloride |
| Bu | butyl |
| CDI | 1,1′-carbonyldiimidazole |
| CH₂Cl₂ or DCM | methylene chloride or 1,2-dichloromethane |
| CH₃CN or MeCN | acetonitrile |
| conc. | concentrated |
| CPME | cyclopentyl methyl ether |
| cPrNH₂ | cyclopropylamine |
| Cs₂CO₃ | cesium carbonate |
| Cu(OTf)₂ | copper(II) trifluoromethansulfonate |
| Cy or CyH | cyclohexane |
| DBU | 1,8-diazabicyclo[5.4.0]undec-7-ene |
| DCE | 1,2-dichloroethane |
| DCM | dichloromethane |
| DIBAL or DIBAL-H | diisobutylaluminium hydride |
| DIEA or DIPEA | diisopropyl ethylamine |
| Dioxane | 1,4-dioxane |
| DMA | N,N-dimethylacetamide |
| DMAP | 4-dimethylaminopyidine |
| DMF | N,N-dimethylformamide |
| DMSO | dimethylsulfoxide |
| Dowtherm ® A | eutectic mixture of 26.5% diphenyl + |
| | 73.5% diphenyl oxide |
| Et | ethyl |
| Et₃N or TEA | triethylamine |
| Et₂O | diethyl ether |
| EtOH | ethanol |
| EtOAc | ethyl acetate |
| h, hr | hour(s) |
| HATU | O-(7-Azabenzotriazol-1yl)-N,N,N′N′- |
| | tetramethylyronium hexafluorophosphate |
| HCl | hydrochloric acid |
| HFIP | 1,1,1,3,3,3-hexafluoro-2-propanol |
| i-Pr₂Net | N′,N′-diisopropylethylamine |
| iPr₂O | diisopropyl ether |
| KI | potassium iodide |
| KOH | potassium hydroxide |
| KOt-Bu or KOtBu | potassium tert-butoxide |
| L | liter(s) |
| LCMS | liquid chromatography-mass spectroscopy |
| LiHDMS | lithium hexamethyldisilazide |
| LiOH | lithium hydroxide |
| Me | methyl |
| MeI | iodomethane |
| MeOH or CH₃OH | methanol |
| Min | minute(s) |
| mL | milliliter(s) |
| MnO₂ | manganese dioxide |
| MS | mass spectrum |
| NaBH₄ | sodium borohydride |
| Na₂CO₃ | sodium carbonate |
| NaH | sodium hydride |
| NaHCO₃ | sodium bicarbonate |
| NaOH | sodium hydroxide |
| Na₂SO₄ | sodium sulfate |
| NCS | N-chlorosuccinimide |
| NH₄Cl | ammonium chloride |
| NH₄OH | ammonium hydroxide |
| NMP | N-methyl-2-pyrrolidone |
| Oxone ® | potassium peroxymonosulfate |
| PdOAc₂ | lead acetate |
| Ph | phenyl |
| PPh₃ | triphenyl phospine |
| POCl₃ | phosphoryl chloride |
| Pr | propyl |
| PyBroP ® | Bromotripyrrolidinophosphonium |
| | hexafluorophosphate |
| Rt | room temperature |
| SOCl₂ | thionyl chloride |
| t-BuOH | tert-butanol |
| TBAF | tetrabutylammonium fluoride |
| TBAI | tetrabutylammonium iodide |
| TBS | tert-butyl(methoxy)dimethylsilyl |
| TEA | triethylamine |
| TFA | trifluoroacetic acid |
| THF | tetrahydrofuran |
| T3P ® | 2,4,6-tripropyl-1,3,5,2,4,6-trioxatriphosphinane |
| | 2,4,6-trioxide |

The compounds useful in this invention can be prepared using intermediate compounds and analogous methods to those disclosed in International Patent Application Publication No. WO2014/125444 and as hereinafter described.

### Preparation 1

### Ethyl 2-ethoxy-2-iminoacetate

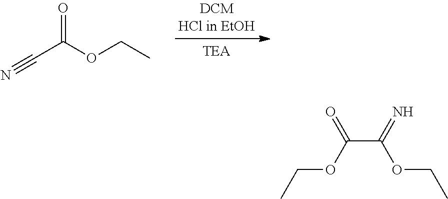

To a solution of ethyl carbonocyanidate (40 g, 404 mmol) in DCM (200 mL) stirred under nitrogen at 0° C. was added a solution of HCl (45 wt. %, 27.3 mL, 404 mmol) in EtOH dropwise over 15 min. The reaction mixture was stirred at 0° C. for 3 hr and allowed to stand overnight at −5° C. to −3° C. To the resulting mixture was added DCM (250 mL) at 0° C. TEA (113 mL, 807 mmol) in DCM (50 mL) was added dropwise over 30 min at 0° C. The mixture was stirred for 30 min at 0° C., and water (100 mL) was added at 0° C. The resulting mixture was stirred for 5 min. The organic layer was separated, dried over sodium sulfate, and evaporated. Diethyl ether (50 mL) was added to the residue and the solid was filtered. The filtrate was dried to afford ethyl 2-ethoxy-2-iminoacetate as a pale yellow liquid (31.0 g, 214 mmol, 52.9% yield). ¹H NMR (400 MHz, CDCl₃) δ 8.78 (s, 1H), 4.36-4.28 (m, 4H), 1.40-1.35 (m, 6H).

### Preparation 2

### Ethyl 2-amino-2-(2-(2-phenylacetyl)hydrazono)acetate

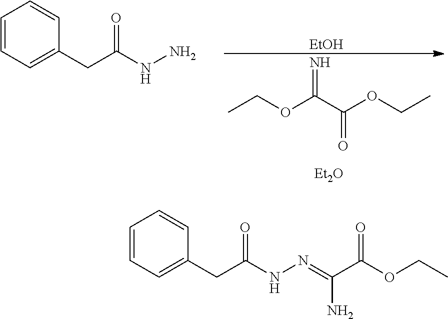

To 2-phenylacetohydrazide (39.5 g, 263 mmol) in ethanol (150 mL) was added ethyl 2-ethoxy-2-iminoacetate (39.5 g, 272 mmol) and diethyl ether (200 mL). The reaction mixture was stirred for 10 min and solid formed. The reaction mixture was stirred for 5 hours and diethyl ether (50 mL) was added. The resulting mixture was stirred for 17 hours. The solid was filtered, rinsed with diethyl ether, and dried to give ethyl 2-amino-2-(2-(2-phenylacetyl)hydrazono)acetate as a white solid (59 g, 85% yield). The filtrate sat for 5 days and additional white solid precipitated out. The solid was filtered and dried to give 2-amino-2-(2-(2-phenylacetyl)hydrazono)acetate as a white solid (4.8 g) (92% total yield). MS ES⁺ m/z 250.1 [M+H]⁺; ¹H NMR (400 MHz, DMSO-d6) δ 9.95 (d, J=17.18 Hz, 1H), 7.13-7.37 (m, 5H), 6.50 (d, 2H), 4.24 (dq, J=7.07, 10.86 Hz, 2H), 3.86 (s, 1H), 3.50 (s, 1H), 1.27 (dt, J=7.07, 17.43 Hz, 3H).

### Preparation 3

### Ethyl 5-benzyl-4H-1,2,4-triazole-3-carboxylate

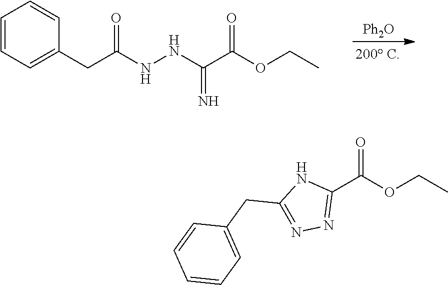

A solution of ethyl 2-imino-2-(2-(2-phenylacetyl)hydrazinyl)acetate (35 g, 140 mmol) in diphenyl ether (300 mL) was stirred for 4 hours under nitrogen at 200° C. The reaction mixture was cooled to rt, diluted with diethyl ether (750 mL), and stirred for 15 minutes. The precipitate was filtered and dried to afford ethyl 5-benzyl-4H-1,2,4-triazole-3-carboxylate as a brown solid (29 g, 105 mmol, 74.6% yield). MS ES⁺ m/z 232.1 [M+H]⁺; ¹H NMR (400 MHz, DMSO-d₆) δ 14.4 (s, 1H), 7.34-7.25 (m, 5H), 4.31-4.26 (m, 2H), 4.13 (s, 2H), 1.28 (t, J=6.8 Hz, 3H).

### Preparation 4

### 5-Benzyl-4H-1,2,4-triazole-3-carboxylic acid

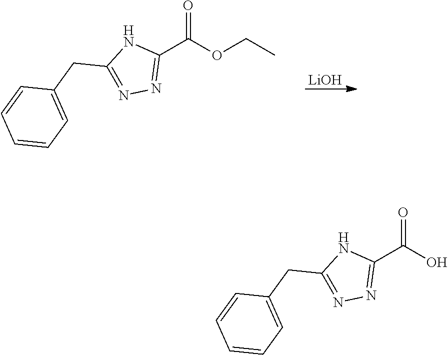

To ethyl 5-benzyl-4H-1,2,4-triazole-3-carboxylate (9.2 g) in water (100 mL) was added 2M aqueous LiOH (60 mL) dropwise over 20 min while maintaining the reaction temperature of about 20° C. The reaction mixture was stirred at 20-25° C. for 3 hrs and then cooled in a MeOH-ice bath to −5° C. 2M HCl (70 mL) was added dropwise over 10 min maintaining the reaction temperature below 5° C. The suspension was stirred at 0° C. for 30 min and the solids were collected by filtration. The solids were washed with ice cold water several times. The filter cake was air-dried on a filter funnel overnight to afford 5-benzyl-4H-1,2,4-triazole-3-carboxylic acid as a white solid (7.5 g, 93% yield). MS ES⁺ m/z 204.4 [M+H]⁺; ¹H NMR (400 MHz, DMSO-d₆) δ 14.33 (s, 1H), 13.13 (br s, 1H), 7.33-7.20 (m, 5H), 4.10-4.03 (m, 2H).

### Preparation 5

### Ethyl 2-amino-2-(2-(2-(2-fluorophenyl)acetyl)hydrazono)acetate

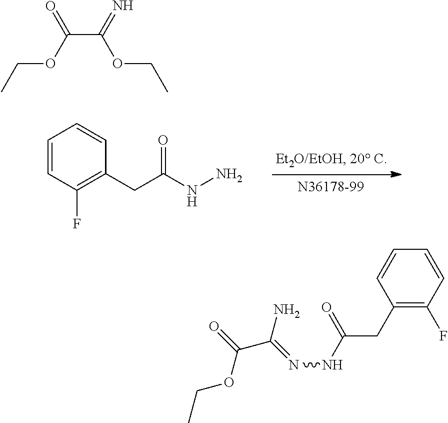

2-(2-fluorophenyl)acetohydrazide (7.05 g, 41.9 mmol) was partially dissolved in ethanol (30 mL), and then ethyl 2-ethoxy-2-iminoacetate (6.39 g, 44.0 mmol) and diethyl ether (35 mL) were added. The reaction mixture was stirred for 0.5 hours, and diethyl ether (100 mL) was added. The resulting mixture was stirred for 18 hours. The solid was filtered off, rinsed with diethyl ether, and dried to give ethyl 2-amino-2-(2-(2-(2-fluorophenyl)acetyl)hydrazono)acetate as an off-white solid (10 g, 89% yield). MS ES⁺ m/z 268 [M+H]⁺; ¹H NMR (400 MHz, DMSO-d₆) δ 9.80-10.25 (m, 1H), 7.25-7.43 (m, 2H), 7.09-7.21 (m, 2H), 6.42-6.60 (m, 2H), 4.23 (dq, J=1.52, 7.07 Hz, 2H), 3.92 (s, 1H), 3.58 (s, 1H), 1.26 (dt, J=5.05, 7.07 Hz, 3H).

### Preparation 6

### Ethyl 5-(2-fluorobenzyl)-4H-1,2,4-triazole-3-carboxylate

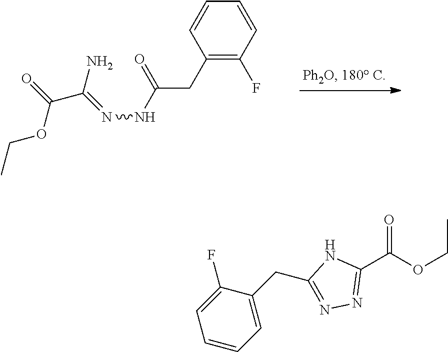 [SMILES: CCOC(=O)C(N)=NNC(=O)Cc1ccccc1F ; CCOC(=O)c1nnc(Cc2ccccc2F)[nH]1]

Ethyl 2-amino-2-(2-(2-(2-fluorophenyl)acetyl)hydrazono)acetate (10 g, 37.4 mmol) was suspended in Dowtherm® A (100 mL), heated at 180° C. for 4.5 hours, and cooled to room temperature. Hexanes (˜200 mL) was added, and the mixture was stirred for 15 minutes. The solid precipitate was filtered, rinsed with hexanes, and dried to give ethyl 5-(2-fluorobenzyl)-4H-1,2,4-triazole-3-carboxylate as a light tan solid (8.51 g, 91% yield). MS ES⁺ m/z 250 [M+H]⁺; ¹H NMR (400 MHz, DMSO-d₆) δ 14.50 (br s, 1H), 7.28-7.43 (m, 2H), 7.13-7.25 (m, 2H), 4.24-4.40 (m, J=6.80 Hz, 2H), 4.17 (br s, 2H), 1.29 (t, J=7.07 Hz, 3H).

### Preparation 7

### 5-(2-Fluorobenzyl)-4H-1,2,4-triazole-3-carboxylic acid hydrochloride

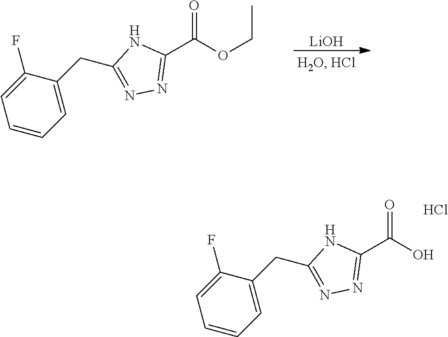

To a suspension of ethyl 5-(2-fluorobenzyl)-4H-1,2,4-triazole-3-carboxylate (8.51 g, 33.1 mmol) in water (60 mL) was added a solution of lithium hydroxide (1.747 g, 72.9 mmol) in water (30 mL) dropwise. The mixture was stirred for 3 days at room temperature and was cooled in an ice water bath. Concentrated HCl (10 mL, 60.0 mmol) was added dropwise until the mixture reached pH ˜3. A solid precipitated from the mixture, and the mixture was stirred for 10 minutes. The precipitate was filtered, rinsed with cold water, and dried in high vacuum for 20 hrs to give 5-(2-fluorobenzyl)-4H-1,2,4-triazole-3-carboxylic acid hydrochloride (7.0 g, 85% yield) as a tan solid. MS ES⁺ m/z 222 [M+H]⁺; ¹H NMR (400 MHz, MeOD-d₄) δ 7.27-7.43 (m, 2H), 7.05-7.23 (m, 2H), 4.23 (s, 2H).

### Example 1

### (S)—N-(9-Fluoro-2-oxo-2,3,4,5-tetrahydro-1H-benzo[b][1,4]diazepin-3-yl)-5-(2-fluorobenzyl)-4H-1,2,4-triazole-3-carboxamide

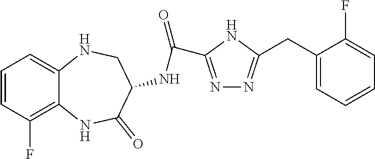 [SMILES: O=C(N[C@H]1CNc2cccc(F)c2NC1=O)c1nnc(Cc2ccccc2F)[nH]1]

Step 1: (S)-2-((tert-Butoxycarbonyl)amino)-3-((3-fluoro-2-nitrophenyl)amino)propanoic acid

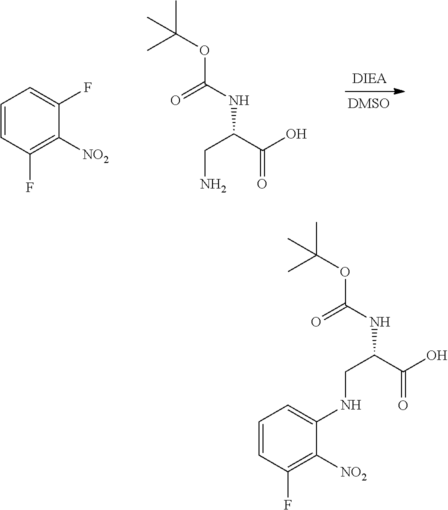 [SMILES: O=[N+]([O-])c1c(F)cccc1F ; CC(C)(C)OC(=O)N[C@@H](CN)C(=O)O ; CC(C)(C)OC(=O)N[C@@H](CNc1cccc(F)c1[N+](=O)[O-])C(=O)O]

To a suspension of (S)-3-amino-2-((tert-butoxycarbonyl)amino)propanoic acid (7.33 g, 35.9 mmol) and 1,3-difluoro-2-nitrobenzene (5.19 g, 32.6 mmol) in DMSO (80 mL), DIEA (19.94 mL, 114 mmol) was added and the mixture was stirred at room temperature for 5 days. The mixture was diluted with 200 mL water and extracted with ether (3×100 mL). The aqueous phase was acidified with 1N HCl (120 mL, 120 mmol) to about pH 2. A deep orange oil separated which was extracted with EtOAc (3×50 mL). The organic layers were combined and washed with water (3×50 mL) and brine (2×50 mL), dried over sodium sulfate, and was concentrated in vacuo to afford (S)-2-((tert-butoxycarbonyl)amino)-3-((3-fluoro-2-nitrophenyl)amino)propanoic acid as a brown residue (10.91 g, 31.8 mmol, 97% yield). MS ES⁺ m/z 244/288/366 for [M-Boc/M-tBu/M+Na]⁺; ¹H NMR (400 MHz, DMSO-d₆) δ ppm

12.92 (br s, 1H), 7.46 (m, 1H), 7.21-7.36 (m, 2H), 6.84 (d, J=8.84 Hz, 1H), 6.63 (dd, J=11.62, 8.08 Hz, 1H), 4.20 (m, 1H), 3.66 (m, 1H), 3.42-3.55 (m, 1H), 1.36 (s, 9H).

### Step 2: (S)-3-((2-Amino-3-fluorophenyl)amino)-2-((tert-butoxycarbonyl)amino)propanoic acid

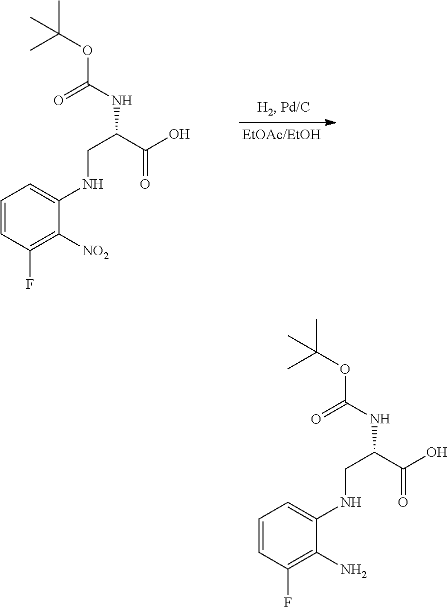 [SMILES: CC(C)(C)OC(=O)N[C@@H](CNc1cccc(F)c1[N+](=O)[O-])C(=O)O ; CC(C)(C)OC(=O)N[C@@H](CNc1cccc(F)c1N)C(=O)O]

A solution of (S)-2-((tert-butoxycarbonyl)amino)-3-((3-fluoro-2-nitrophenyl)amino)propanoic acid (10.91 g, 31.8 mmol) in ethyl acetate (63.6 ml) and ethanol (63.6 ml) was hydrogenated in the presence of 10% Pd/C (1.09 g, 1.024 mmol) at 35 psi for 2 hours in a Parr shaker. The catalyst was filtered off. The solution was evaporated and co-evaporated with toluene to afford (S)-3-((2-amino-3-fluorophenyl)amino)-2-((tert-butoxycarbonyl)amino)propanoic acid as a brown solid foam (9.96 g, 31.8 mmol, 100% yield). MS ES⁺ m/z 314 [M+H]⁺; ¹H NMR (400 MHz, DMSO-d₆) δ ppm 6.18-6.64 (m, 3H), 4.21 (m, 1H), 3.22-3.54 (m, 4H), 1.35 (s, 9H). This material was used in Step 3 without further purification.

### Step 3: (S)-tert-Butyl (9-fluoro-2-oxo-2,3,4,5-tetrahydro-1H-benzo[b][1,4]diazepin-3-yl)carbamate

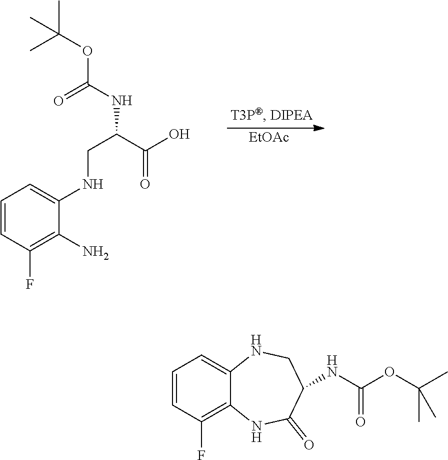 [SMILES: "EtOAc" ; CC(C)(C)OC(=O)N[C@@H](CNc1cccc(F)c1N)C(=O)O ; CC(C)(C)OC(=O)N[C@H]1CNc2cccc(F)c2NC1=O]

A solution of (S)-3-((2-amino-3-fluorophenyl)amino)-2-((tert-butoxycarbonyl)amino)propanoic acid (9.86 g, 31.5 mmol) in ethyl acetate (100 mL) was submersed in an ice-water bath. To this solution was added DIEA (16.49 mL, 94 mmol) followed by 50% T3P® in ethyl acetate (28.1 mL, 47.2 mmol). The mixture was washed with water and brine, dried over sodium sulfate, and evaporated in vacuo to provide 7.55 g of crude product. The crude product was purified by normal phase column chromatography (silica gel: 220 g column; eluent: eluent A=hexanes, eluent B=(EtOAc/EtOH 3/1), 0-60% B gradient) to afford (S)-tert-butyl (9-fluoro-2-oxo-2,3,4,5-tetrahydro-1H-benzo[b][1,4]diazepin-3-yl)carbamate as a pale yellow solid foam (5.99 g, 20.28 mmol, 64.5% yield). MS ES⁺ m/z 196 [M-Boc+H]⁺; ¹H NMR (400 MHz, DMSO-d₆) δ ppm 9.53 (s, 1H), 6.90-7.04 (m, 2H), 6.59-6.75 (m, 2H), 5.87 (d, J=5.56 Hz, 1H), 4.20 (m, 1H), 3.52 (m, 1H), 3.38 (t, J=11.12 Hz, 1H), 1.37 (s, 9H).

### Step 4: (S)-3-Amino-9-fluoro-4,5-dihydro-1H-benzo[b][1,4]diazepin-2(3H)-one dihydrochloride

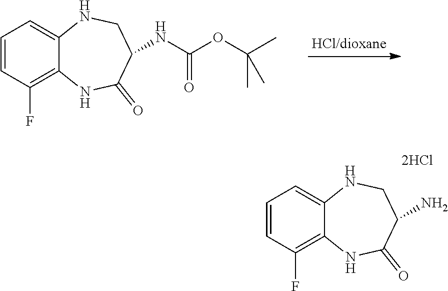 [SMILES: CC(C)(C)OC(=O)N[C@H]1CNc2cccc(F)c2NC1=O ; N[C@H]1CNc2cccc(F)c2NC1=O]

A solution of (S)-tert-butyl (9-fluoro-2-oxo-2,3,4,5-tetrahydro-1H-benzo[b][1,4]diazepin-3-yl)carbamate (1.5 g, 5.08 mmol) in 4M HCl/dioxane (30 ml, 120 mmol) was stirred at room temperature for 7 hrs. The reaction mixture was evaporated (co-evaporated with MeOH and ether). The resulting residue was dried to afford (S)-3-amino-9-fluoro-4,5-dihydro-1H-benzo[b][1,4]diazepin-2(3H)-one dihydrochloride as a pale yellow solid (1.523 g, 5.21 mmol, 100% yield). MS ES⁺ m/z 196 [M+H]⁺; ¹H NMR (400 MHz, DMSO-d₆) δ ppm 10.02 (s, 1H), 8.55 (d, J=4.04 Hz, 3H), 6.99 (td, J=8.15, 6.44 Hz, 1H), 6.72 (d, J=8.08 Hz, 1H), 6.61-6.69 (m, 1H), 4.09-4.24 (m, 1H), 3.82 (dd, J=11.12, 4.29 Hz, 1H), 3.48 (t, J=10.99 Hz, 1H).

### Step 5: Ethyl 2-amino-2-(2-(2-(2-fluorophenyl)acetyl)hydrazono)acetate

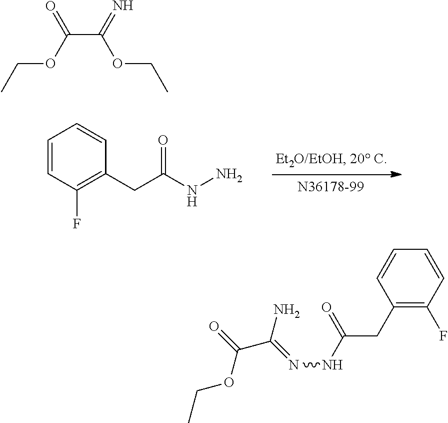

2-(2-fluorophenyl)acetohydrazide (7.05 g, 41.9 mmol) was partially dissolved in ethanol (30 mL), and then ethyl 2-ethoxy-2-iminoacetate (6.39 g, 44.0 mmol) and diethyl ether (35 mL) were added. The reaction mixture was stirred for 0.5 hours, and diethyl ether (100 mL) was added. The resulting mixture was stirred for 18 hours. The solid was filtered off, rinsed with diethyl ether, and dried to give ethyl 2-amino-2-(2-(2-(2-fluorophenyl)acetyl)hydrazono)acetate as an off-white solid (10 g, 89% yield). MS ES⁺ m/z 268 [M+H]⁺; ¹H NMR (400 MHz, DMSO-d₆) δ 9.80-10.25 (m, 1H), 7.25-7.43 (m, 2H), 7.09-7.21 (m, 2H), 6.42-6.60 (m, 2H), 4.23 (dq, J=1.52, 7.07 Hz, 2H), 3.92 (s, 1H), 3.58 (s, 1H), 1.26 (dt, J=5.05, 7.07 Hz, 3H).

### Step 6: Ethyl 5-(2-fluorobenzyl)-4H-1,2,4-triazole-3-carboxylate

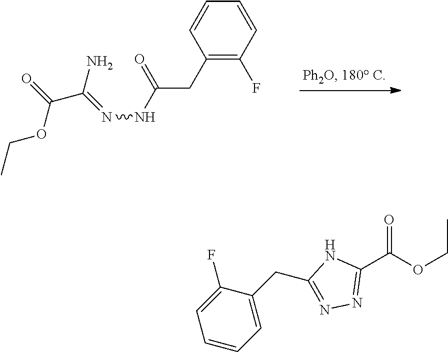 [SMILES: CCOC(=O)C(N)=NNC(=O)Cc1ccccc1F ; CCOC(=O)c1nnc(Cc2ccccc2F)[nH]1]

Ethyl 2-amino-2-(2-(2-(2-fluorophenyl)acetyl)hydrazono)acetate (10 g, 37.4 mmol) was suspended in Dowtherm® A (100 mL), heated at 180° C. for 4.5 hours, and cooled to room temperature. Hexanes (˜200 mL) was added, and the mixture was stirred for 15 minutes. The solid precipitate was filtered, rinsed with hexanes, and dried to give ethyl 5-(2-fluorobenzyl)-4H-1,2,4-triazole-3-carboxylate as a light tan solid (8.51 g, 91% yield). MS ES⁺ m/z 250 [M+H]⁺; ¹H NMR (400 MHz, DMSO-d₆) δ 14.50 (br s, 1H), 7.28-7.43 (m, 2H), 7.13-7.25 (m, 2H), 4.24-4.40 (m, J=6.80 Hz, 2H), 4.17 (br s, 2H), 1.29 (t, J=7.07 Hz, 3H).

### Step 7: 5-(2-Fluorobenzyl)-4H-1,2,4-triazole-3-carboxylic acid hydrochloride

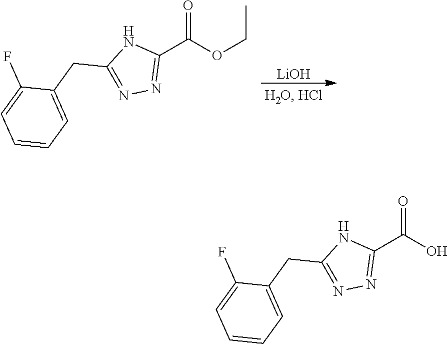

To a suspension of ethyl 5-(2-fluorobenzyl)-4H-1,2,4-triazole-3-carboxylate (8.51 g, 33.1 mmol) in water (60 mL) was added a solution of lithium hydroxide (1.747 g, 72.9 mmol) in water (30 mL) dropwise. The mixture was stirred for 3 days at room temperature and was cooled in an ice water bath. Concentrated HCl (10 mL, 60.0 mmol) was added dropwise until the mixture reached pH ˜3. A solid precipitated from the mixture, and the mixture was stirred for 10 minutes. The precipitate was filtered, rinsed with cold water, and dried in high vacuum for 20 hrs to give 5-(2-fluorobenzyl)-4H-1,2,4-triazole-3-carboxylic acid hydrochloride (7.0 g, 85% yield) as a tan solid. MS ES⁺ m/z 222 [M+H]⁺; ¹H NMR (400 MHz, MeOH-d₄) δ 7.27-7.43 (m, 2H), 7.05-7.23 (m, 2H), 4.23 (s, 2H).

### Step 8: (S)—N-(9-Fluoro-2-oxo-2,3,4,5-tetrahydro-1H-benzo[b][1,4]diazepin-3-yl)-5-(2-fluorobenzyl)-4H-1,2,4-triazole-3-carboxamide

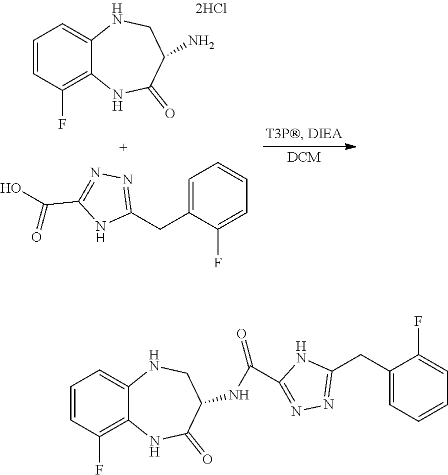 [SMILES: N[C@H]1CNc2cccc(F)c2NC1=O ; O=C(O)c1nnc(Cc2ccccc2F)[nH]1 ; O=C(N[C@H]1CNc2cccc(F)c2NC1=O)c1nnc(Cc2ccccc2F)[nH]1]

A suspension of (S)-3-amino-9-fluoro-4,5-dihydro-1H-benzo[b][1,4]diazepin-2(3H)-one dihydrochloride (200 mg, 0.685 mmol) and 5-(2-fluorobenzyl)-4H-1,2,4-triazole-3-carboxylic acid (216 mg, 0.753 mmol) in dichloromethane (4 mL) was submersed in an ice-water bath. To this suspension was added DIEA (0.717 mL, 4.11 mmol). The mixture was stirred for 15 min. 50% T3P® in EtOAc (0.611 mL, 1.027 mmol) was added dropwise, and the reaction mixture was stirred for 5 min. The mixture was diluted with 10 mL EtOAc, washed with water and brine, dried over sodium sulfate, and was evaporated. The crude material was purified by normal phase column chromatography (silica gel: 40 g column; eluent: eluent A=hexanes, eluent B=(EtOAc/EtOH 3/1), 0-70% B gradient). The pooled clean fractions were evaporated in vacuo, and the residue was triturated with ether. The solid was filtered and dried for 40 hr in high vacuum at 70° C. to give (S)—N-(9-fluoro-2-oxo-2,3,4,5-tetrahydro-1H-benzo[b][1,4]diazepin-3-yl)-5-(2-fluorobenzyl)-4H-1,2,4-triazole-3-carboxamide. MS ES⁺ m/z 399 [M+H]⁺; ¹H NMR (400 MHz, DMSO-d₆) δ ppm 14.63 (br s, 1H), 9.79 (s, 1H), 8.35 (br s, 1H), 7.27-7.41 (m, 2H), 7.19 (d, J=8.08 Hz, 2H), 6.90-7.01 (m, 1H), 6.67 (d, J=8.08 Hz, 1H), 6.60 (t, J=9.09 Hz, 1H), 6.24 (d, J=5.56 Hz, 1H), 4.57-4.69 (m, 1H), 4.16 (br s, 2H), 3.63-3.74 (m, 1H), 3.47 (t, J=9.85 Hz, 1H).

### Example 2

### (S)-5-Benzyl-N-(7-chloro-2-oxo-2,3,4,5-tetrahydro-1H-benzo[b]azepin-3-yl)-4H-1,2,4-triazole-3-carboxamide

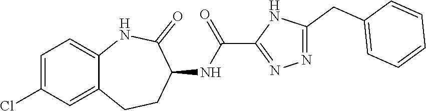 [SMILES: O=C(N[C@H]1CCc2cc(Cl)ccc2NC1=O)c1nnc(Cc2ccccc2)[nH]1]

### Step 1: (S)-5-Benzyl-N-(2-oxo-2,3,4,5-tetrahydro-1H-benzo[b]azepin-3-yl)-4H-1,2,4-triazole-3-carboxamide

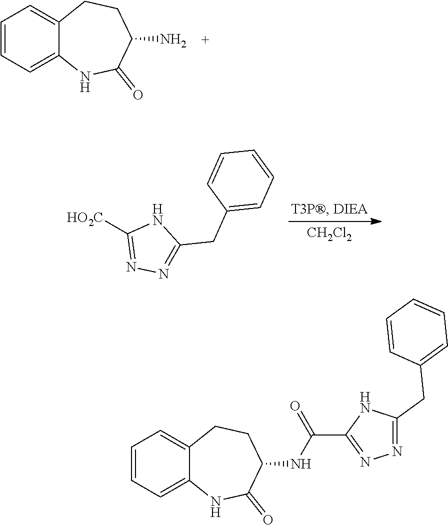

To a solution of (S)-3-amino-4,5-dihydro-1H-benzo[b]azepin-2(3H)-one (50 g, 284 mmol) and 5-benzyl-4H-1,2,4-triazole-3-carboxylic acid (72.1 g, 355 mmol) in DCM (1500 ml) was added DIPEA (173 ml, 993 mmol) at 15° C. The reaction mixture was stirred for 20 minutes and 2,4,6-tripropyl-1,3,5,2,4,6-trioxatriphosphinane-2,4,6-trioxide (50 wt % T3P® in EtOAc, 236 ml, 397 mmol) was slowly added at 15° C. The reaction was stirred overnight. The resulting solid was filtered, and the solid was washed with DCM. The solid was dried under vacuum at 50° C. overnight. The filtrate was concentrated under reduced pressure. To the resulting residue was added cold water. The mixture was stirred and a white solid precipitated out of solution. The white solid was collected and washed with water and ethyl ether. The solid was dried under vacuum at 50° C. for 3 days to afford (S)-5-benzyl-N-(2-oxo-2,3,4,5-tetrahydro-1H-benzo[b]azepin-3-yl)-4H-1,2,4-triazole-3-carboxamide (102 g, 282 mmol, 99% yield). ¹H NMR (MeOD-d₄) δ: 7.18-7.48 (m, 8H), 7.10 (d, J=7.6 Hz, 1H), 4.58 (m, 1H), 4.17 (s, 2H), 2.97 (m 1H), 2.77 (m, 1H), 2.67 (m, 1H), 2.23 (m, 1H). MS ES⁺ m/z 362 [M+H]⁺.

### Step 2: (S)-5-Benzyl-N-(7-chloro-2-oxo-2,3,4,5-tetrahydro-1H-benzo[b]azepin-3-yl)-4H-1,2,4-triazole-3-carboxamide

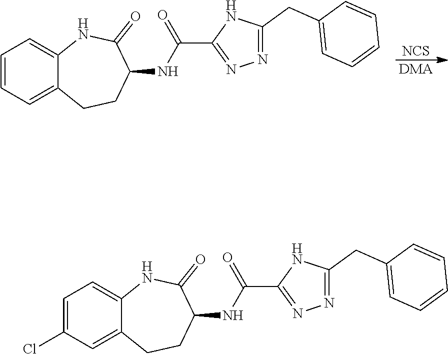

To a solution of (S)-5-benzyl-N-(2-oxo-2,3,4,5-tetrahydro-1H-benzo[b]azepin-3-yl)-4H-1,2,4-triazole-3-carboxamide (35 g, 97 mmol) in DMA (700 ml) was added NCS (14.87 g, 111 mmol) at 0° C. The reaction mixture was stirred for 30 min, warmed to room temperature, and stirred for 5 hrs. A second portion of NCS (3.88 g, 29.1 mmol) was added to the reaction mixture. The resulting mixture was stirred for an additional 24 hrs. A third portion of NCS (1.293 g, 9.68 mmol) was added. The resulting mixture was stirred at room temperature for 16 h. The reaction was then quenched with cold water. The white solid was collected by filtration and washed with water 3 times to provide (S)-5-benzyl-N-(7-chloro-2-oxo-2,3,4,5-tetrahydro-1H-benzo[b]azepin-3-yl)-4H-1,2,4-triazole-3-carboxamide (36 g, 91 mmol, 94% yield). The product was air dried overnight. Additional purification was achieved by suspending (S)-5-benzyl-N-(7-chloro-2-oxo-2,3,4,5-tetrahydro-1H-benzo[b]azepin-3-yl)-4H-1,2,4-triazole-3-carboxamide (10 g, 25.3 mmol) in hot methanol (500 mL) for 1 h. The solution was then cooled to room temperature and filtered. The solid was washed with methanol (2×75 mL) to give (S)-5-benzyl-N-(7-chloro-2-oxo-2,3,4,5-tetrahydro-1H-benzo[b]azepin-3-yl)-4H-1,2,4-triazole-3-carboxamide (7 g, 70% yield). MS ES⁺ m/z 396 and 398 [M+H]⁺; ¹H NMR (DMSO-d₆) δ: 10.06 (s, 1H), 8.31 (br s, 1H), 7.44 (d, J=2.5 Hz, 1H), 7.18-7.40 (m, 7H), 7.05 (d, J=8.6 Hz, 1H), 4.32 (dt, J=11.5, 7.9 Hz, 1H), 4.11 (s, 2H), 2.63-2.80 (m, 2H), 2.37-2.49 (m, 1H), 2.25 (br s, 1H).

### Example 3

### (S)-5-(2-Fluorobenzyl)-N-(1-methyl-2-oxo-2,3,4,5-tetrahydro-1H-benzo[b][1,4]diazepin-3-yl)-1H-1,2,4-triazole-3-carboxamide

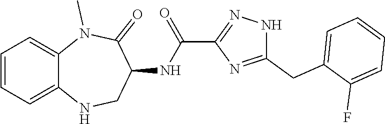 [SMILES: CN1C(=O)[C@@H](NC(=O)c2n[nH]c(Cc3ccccc3F)n2)CNc2ccccc21]

### Step 1: (S)-2-((tert-Butoxycarbonyl) amino)-3-((2-nitrophenyl)amino)propanoic acid

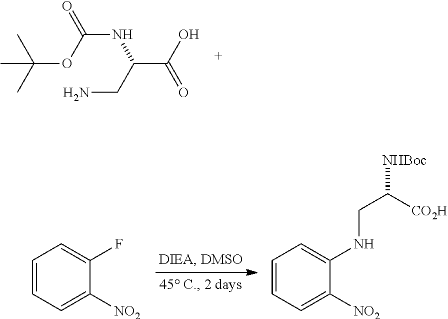

To a suspension of (S)-3-amino-2-((tert-butoxycarbonyl)amino)propanoic acid (200 g, 979 mmol) and 1-fluoro-2-nitrobenzene (138 g, 979 mmol) in DMF (2000 mL) stirred under nitrogen at room temp was added sodium bicarbonate (247 g, 2938 mmol). The reaction mixture was stirred at 70° C. for 24 hr. Water was then added (8 L). The aqueous layer was washed with diethyl ether (2×2 L), acidified with citric acid to pH<5, and washed with EtOAc (2×2 L). The organic layers were combined. The combined organic layers were washed with water (2×2 L), washed with brine (2 L), dried over anhydrous Na₂SO₄, filtered, and concentrated to afford (S)-2-((tert-butoxycarbonyl)amino)-3-((2-nitrophenyl)amino)propanoic acid as reddish yellow solid (201 g, 615 mmol, 62.8% yield). MS ES⁺ m/z 326 [M+H]⁺; ¹H NMR (DMSO-d₆) δ 12.90 (br s, 1H), 8.15-8.26 (m, 1H), 8.07 (br d, J=8.6 Hz, 1H), 7.57 (br t, J=7.7 Hz, 1H), 7.30 (br d, J=7.7 Hz, 1H), 7.09 (d, J=8.6 Hz, 1H), 6.73 (t, J=7.7 Hz, 1H), 4.15-4.30 (m, 1H), 3.67-3.86 (m, 1H), 3.54 (ddd, J=14.0, 8.7, 5.8 Hz, 1H), 1.2-1.34 (m, 9H).

### Step 2: (S)-3-((2-Aminophenyl)amino)-2-((tert-butoxycarbonyl)amino)propanoic acid

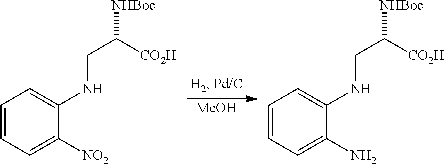 [SMILES: "MeOH" ; CC(C)(C)OC(=O)N[C@@H](CNc1ccccc1[N+](=O)[O-])C(=O)O ; CC(C)(C)OC(=O)N[C@@H](CNc1ccccc1N)C(=O)O]

To a solution of (S)-2-((tert-butoxycarbonyl)amino)-3-((2-nitrophenyl)amino)propanoic acid (80 g, 246 mmol) in methanol (1000 mL) in a Parr-shaker under nitrogen at room temp was added 10% Pd/C (50% wet) (10.47 g, 9.84 mmol). The reaction mixture was hydrogenated under 60 psi at 25° C. for 5 hr. The reaction mixture was filtered through a celite pad, the celite pad was washed with methanol (300 mL) followed by 10% MeOH in DCM (2×300 mL). The filtrate was concentrated under reduced pressure to afforded (5)-3-((2-aminophenyl)amino)-2-((tertbutoxycarbonyl)amino)propanoic acid (75 g, 216 mmol, 88% yield) as a brown solid. MS ES⁺ m/z 296 [M+H]⁺; ¹H NMR (DMSO-d₆) δ 7.11 (br d, J=8.1 Hz, 1H), 6.63-6.85 (m, 1H), 6.4-6.62 (m, 5H), 4.10-4.27 (m, 1H), 3.24-3.45 (m, 4H), 1.28-1.35 (m, 9H).

### Step 3: (S)-tert-Butyl (2-oxo-2,3,4,5-tetrahydro-1H-benzo[b][1,4]diazepin-3-yl)carbamate

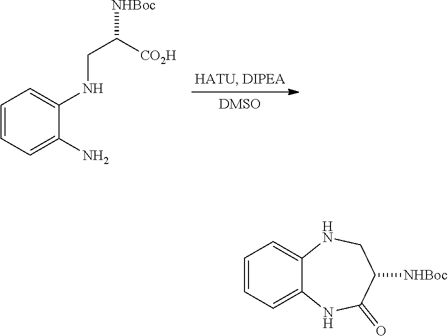 [SMILES: CC(C)(C)OC(=O)N[C@@H](CNc1ccccc1N)C(=O)O ; CC(C)(C)OC(=O)N[C@H]1CNc2ccccc2NC1=O]

To a solution of (S)-3-((2-aminophenyl)amino)-2-((tert-butoxycarbonyl)amino)propanoic acid (150 g, 423 mmol) in DMSO (1500 mL) was added DIPEA (185 mL, 1058 mmol) and HATU (633 g, 1666 mmol). The resulting mixture was stirred at 25° C. for 3 hr, cooled in an ice-water bath, and diluted with cold water (5 L, added over ˜10 minutes) with vigorous stirring. The organics were extracted with EtOAc (2×3 L). The combined organics were washed with water (2 L) and brine (2 L). The organic layer was dried over anhydrous Na₂SO₄, filtered, and concentrated in vacuo to give a residue (140 g). The residue was purified by normal phase column chromatography (60-120 mesh silica gel column; eluent: 10-40% EtOAc in hexanes) to afforded (S)-tert-butyl (2-oxo-2,3,4,5-tetrahydro-1H-benzo[b][1,4]diazepin-3-yl)carbamate as a pale brown solid (100 g, 341 mmol, 81% yield). MS ES⁺ m/z 278 [M+H]⁺; ¹H NMR (DMSO-d₆) δ 9.67 (s, 1H), 6.87-6.94 (m, 2H), 6.77-6.86 (m, 2H), 6.69-6.76 (m, 1H), 5.59 (br d, J=5.5 Hz, 1H), 4.15 (br t, J=11.3 Hz, 1H), 3.49 (dt, J=10.7, 5.5 Hz, 1H), 3.31-3.37 (m, 1H), 1.37 (s, 9H).

### Step 4: (S)-tert-Butyl (1-methyl-2-oxo-2,3,4,5-tetrahydro-1H-benzo[b][1,4]diazepin-3-yl)carbamate

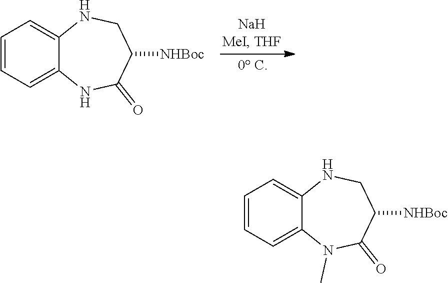 [SMILES: CC(C)(C)OC(=O)N[C@H]1CNc2ccccc2NC1=O ; CN1C(=O)[C@@H](NC(=O)OC(C)(C)C)CNc2ccccc21]

To a suspension of 60% NaH in mineral oil (7.93 g, 198 mmol) in THF (300 mL) stirred under nitrogen at 25° C. was added a solution of (S)-tert-butyl (2-oxo-2,3,4,5-tetrahydro-1H-benzo[b][1,4]diazepin-3-yl)carbamate (50 g, 180 mmol) in THF (200 mL) dropwise over 5 min. The reaction mixture was stirred at 25° C. for 1 hr and then iodomethane (11.84 mL, 189 mmol) was added dropwise over 2 min. Then resulting reaction mixture was stirred at 25° C. for 48 hr and then water (1000 mL) was added. The reaction mixture was extracted with EtOAc (3×500 mL), and combined organic layers were dried over anhydrous Na₂SO₄, filtered, and concentrated in vacuo to afford (S)-tert-butyl (1-methyl-2-oxo-2,3,4,5-tetrahydro-1H-benzo[b][1,4]diazepin-3-yl)carbamate as dark brown gum-like material (56 g, 111 mmol, 61.7% yield). This material was used the next step without further purification. MS ES⁺ m/z 292 [M+H]⁺.

### Step 5: (S)-3-Amino-1-methyl-4,5-dihydro-1H-benzo[b][1,4]diazepin-2(3H)-one dihydrochloride

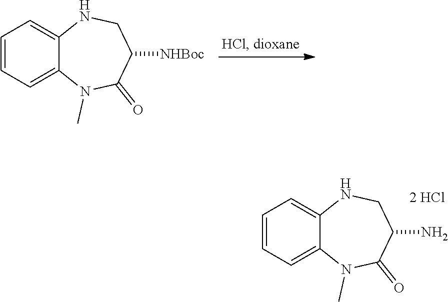 [SMILES: CN1C(=O)[C@@H](NC(=O)OC(C)(C)C)CNc2ccccc21 ; CN1C(=O)[C@@H](N)CNc2ccccc21]

To a solution of (S)-tert-butyl (1-methyl-2-oxo-2,3,4,5-tetrahydro-1H-benzo[b][1,4]diazepin-3-yl)carbamate (179 g, 544 mmol) in DCM (1500 mL) was added 4M HCl (680 mL, 2719 mmol) in 1,4-dioxane at 0° C. The reaction mixture was stirred at 25° C. for 24 hr and then was concentrated in vacuo. The resulting solid was triturated with diethyl ether (600 mL), filtered, washed with diethyl ether (500 mL), and dried under vacuum to afford (S)-3-amino-1-methyl-4,5-dihydro-1H-benzo[b][1,4]diazepin-2 (3H)-one dihydrochloride as an off-white solid (151 g, 526 mmol, 97% yield). MS ES⁺ m/z 192 [M+H]⁺; ¹H NMR (DMSO-d₆) δ 8.40-8.60 (m, 5H), 7.40 (dd, J=7.8, 1.4 Hz, 1H), 7.12-7.30 (m, 3H), 3.96-4.07 (m, 1H), 3.90 (dd, J=10.2, 6.5 Hz, 1H), 3.47-3.58 (m, 1H), 3.31 (s, 3H).

### Step 6: (S)-5-(2-Fluorobenzyl)-N-(1-methyl-2-oxo-2,3,4,5-tetrahydro-1H-benzo[b][1,4]diazepin-3-yl)-1H-1,2,4-triazole-3-carboxamide

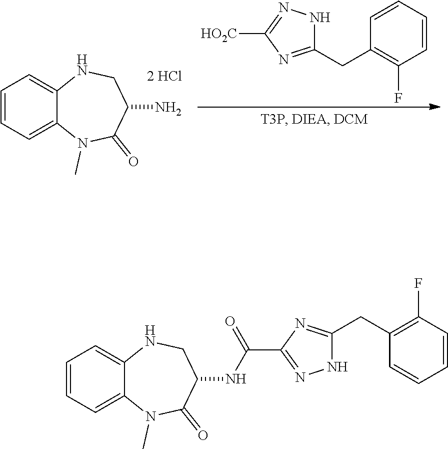 [SMILES: CN1C(=O)[C@@H](N)CNc2ccccc21 ; O=C(O)c1n[nH]c(Cc2ccccc2F)n1 ; CN1C(=O)[C@@H](NC(=O)c2n[nH]c(Cc3ccccc3F)n2)CNc2ccccc21]

To a mixture of (S)-3-amino-1-methyl-4,5-dihydro-1H-benzo[b][1,4]diazepin-2(3H)-one dihydrochloride (100 g, 379 mmol) and 5-(2-fluorobenzyl)-4H-1,2,4-triazole-3-carboxylic acid (80 g, 360 mmol) in DCM (2000 mL) was added DIPEA (331 mL, 1893 mmol) and a ≥50 wt. % solution of T3P in EtOAc (338 mL, 568 mmol) at 0° C. Then resulting mixture was stirred at room temperature for 16 hr. The reaction was diluted with water (2000 mL) and extracted with DCM (2000 mL). The organic layer was washed with water (2×1500 mL) and brine (1×1500 mL), dried over anhydrous Na₂SO₄, filtered, and concentrated in vacuo to afforded (S)-5-(2-fluorobenzyl)-N-(1-methyl-2-oxo-2,3,4,5-tetrahydro-1H-benzo[b][1,4]diazepin-3-yl)-4H-1,2,4-triazole-3-carboxamide as brown solid (112 g, 257 mmol, 68.0% yield). MS ES⁺ m/z 395 [M+H]⁺; ¹H NMR (DMSO-d₆) δ 8.25 (br d, J=7.0 Hz, 1H), 7.24-7.40 (m, 3H), 7.04-7.23 (m, 3H), 6.90-7.04 (m, 2H), 5.38 (br d, J=5.5 Hz, 1H), 4.54-4.69 (m, 1H), 4.14 (s, 2H), 3.68 (dt, J=9.9, 5.8 Hz, 1H), 3.43-3.52 (m, 1H), 3.33-3.40 (m, 1H), 3.27 (s, 3H).

### Example 4

### (S)-5-Benzyl-N-(6,8-difluoro-5-methyl-4-oxo-2,3,4,5-tetrahydrobenzo[b][1,4]oxazepin-3-yl)-1H-1,2,4-triazole-3-carboxamide

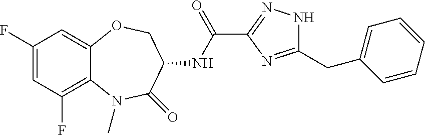 [SMILES: CN1C(=O)[C@@H](NC(=O)c2n[nH]c(Cc3ccccc3)n2)COc2cc(F)cc(F)c21]

### Step 1: Ethyl 2-ethoxy-2-iminoacetate

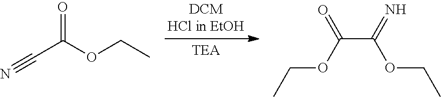

To a solution of ethyl carbonocyanidate (40 g, 404 mmol) in DCM (200 mL) stirred under nitrogen at 0° C. was added a solution of HCl (45 wt %, 27.3 mL, 404 mmol) in EtOH dropwise over 15 min. The reaction mixture was stirred at 0° C. for 3 hr and allowed to stand overnight at −5° C. to 3° C. To the resulting mixture was added DCM (250 mL) at 0° C. TEA (113 mL, 807 mmol) in DCM (50 mL) was added dropwise over 30 min at 0° C. The mixture was stirred for 30 min at 0° C., and water (100 mL) was added at 0° C. The resulting mixture was stirred for 5 min. The organic layer was separated, dried over sodium sulfate, and evaporated. Diethyl ether (50 mL) was added to the residue and the solid was filtered. The filtrate was dried to afford ethyl 2-ethoxy-2-iminoacetate as a pale yellow liquid (31.0 g, 214 mmol, 52.9% yield). ¹H NMR (400 MHz, CDCl₃) δ 8.78 (s, 1H), 4.36-4.28 (m, 4H), 1.40-1.35 (m, 6H).

### Step 2: Ethyl 2-amino-2-(2-(2-phenylacetyl)hydrazono)acetate

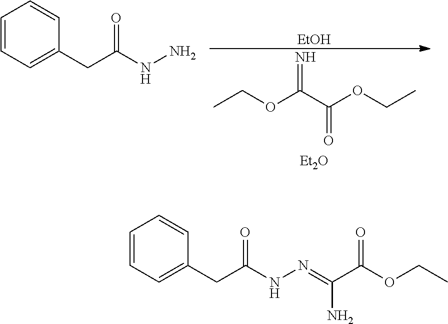

To 2-phenylacetohydrazide (39.5 g, 263 mmol) in ethanol (150 mL) was added ethyl 2-ethoxy-2-iminoacetate (39.5 g, 272 mmol) and diethyl ether (200 mL). The reaction mixture was stirred for 10 min and solid formed. The reaction mixture was stirred for 5 hours and diethyl ether (50 mL) was added. The resulting mixture was stirred for 17 hours. The solid was filtered, rinsed with diethyl ether, and dried to give ethyl 2-amino-2-(2-(2-phenylacetyl)hydrazono)acetate as a white solid (59 g, 85% yield). The filtrate sat for 5 days and additional white solid precipitated out. The solid was filtered and dried to give 2-amino-2-(2-(2-phenylacetyl)hydrazono)acetate as a white solid (4.8 g) (92% total yield). MS ES⁺ m/z 250.1 [M+H]⁺; ¹H NMR (400 MHz, DMSO-d₆) δ 9.95 (d, J=17.18 Hz, 1H), 7.13-7.37 (m, 5H), 6.50 (d, 2H), 4.24 (dq, J=7.07, 10.86 Hz, 2H), 3.86 (s, 1H), 3.50 (s, 1H), 1.27 (dt, J=7.07, 17.43 Hz, 3H).

### Step 3: Ethyl 5-benzyl-4H-1,2,4-triazole-3-carboxylate

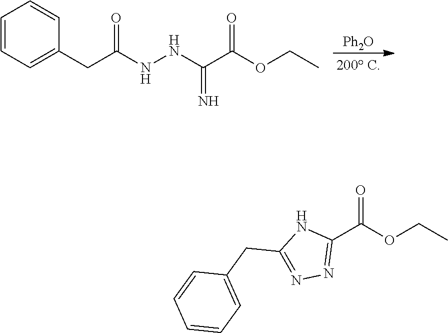

A solution of ethyl 2-imino-2-(2-(2-phenylacetyl)hydrazinyl)acetate (28 g, 120 mmol) in diphenyl ether (250 mL) was stirred for 4 hours under nitrogen at 200° C. Reaction progress was monitored by TLC which showed absence of starting material in 4 h. The reaction mixture was cooled to rt, diluted with diethyl ether (750 mL), and stirred for 15 minutes. The precipitate was filtered and dried to afford ethyl 5-benzyl-4H-1,2,4-triazole-3-carboxylate as an off-white solid (25 g, 106 mmol, 89% yield). MS ES⁺ m/z 232.1 [M+H]⁺; ¹H NMR (400 MHz, DMSO-d₆) δ 14.4 (s, 1H), 7.34-7.25 (m, 5H), 4.31-4.26 (m, 2H), 4.13 (s, 2H), 1.28 (t, J=6.8 Hz, 3H).

### Step 4: 5-Benzyl-4H-1,2,4-triazole-3-carboxylic acid

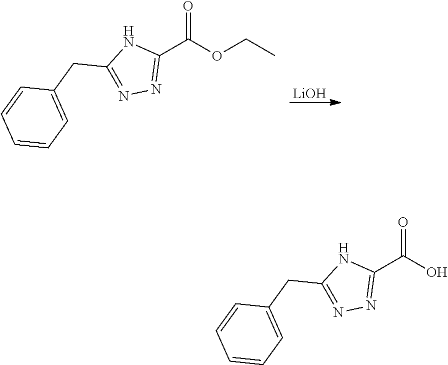

To ethyl 5-benzyl-4H-1,2,4-triazole-3-carboxylate (9.2 g) in water (100 mL) was added 2M aqueous LiOH (60 mL) dropwise over 20 min while maintaining the reaction temperature of about 20° C. The reaction mixture was stirred at 20-25° C. for 3 hrs and then cooled in a MeOH-ice bath to −5° C. 2M HCl (70 mL) was added dropwise over 10 min maintaining the reaction temperature below 5° C. The suspension was stirred at 0° C. for 30 min and the solids were collected by filtration. The solids were washed with ice cold water several times. The filter cake was air-dried on a filter funnel overnight to afford 5-benzyl-4H-1,2,4-triazole-3-carboxylic acid as a white solid (7.5 g, 93% yield). MS ES⁺ m/z 204.4 [M+H]⁺; ¹H NMR (400 MHz, DMSO-d₆) δ 14.33 (s, 1H), 13.13 (br s, 1H), 7.33-7.20 (m, 5H), 4.10-4.03 (m, 2H).

### Step 5: (S)-2-(tert-Butoxycarbonylamino)-3-hydroxypropanoic acid

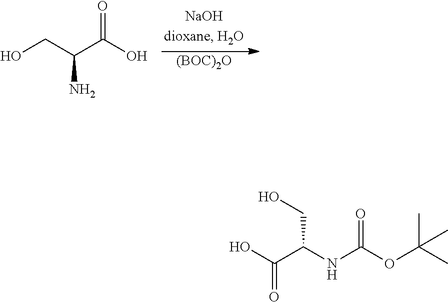

To a stirred suspension of (S)-2-amino-3-hydroxypropanoic acid (100 g, 952 mmol, 1.0 eq) in dioxane (600 mL) and water (600 mL) under nitrogen at 0° C. was added NaOH (76 g, 1903 mmol, 2.0 eq) dropwise. The reaction mixture was stirred for 10 min. Boc anhydride (221 mL, 952 mmol, 1.0 eq) was added dropwise over 10 min. The reaction mixture was stirred at ambient temperature for 16 h. The reaction mixture was acidified with 1.0 N HCl to pH 2. The reaction mixture was extracted with ethyl acetate (3×500 mL). The organic phase was washed with brine (500 mL), dried over anhydrous sodium sulfate, and concentrated in vacuo to afford (S)-2-((tertbutoxycarbonyl)amino)-3-hydroxypropanoic acid as a colorless gum-like material (170 g, 78% yield). MS ES⁺ m/z 205.9 [M+H]⁺; ¹H NMR (400 MHz, DMSO-d₆) δ 6.72 (d, J=8.4 Hz, 1H), 4.00-3.94 (m, 1H), 3.62 (d, J=4.8 Hz, 2H), 1.38 (s, 9H).

### Step 6: (S)-2-(tert-Butoxycarbonylamino)-3-(3,5-difluoro-2-nitrophenoxy)propanoic acid

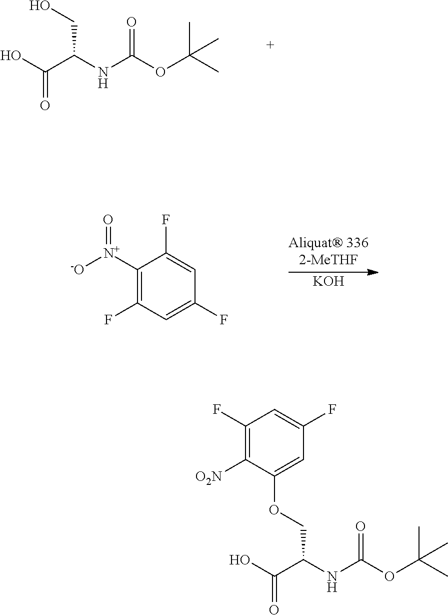

To a stirred solution of Aliquat 336 (13 g) in 2-MeTHF (100 mL) was added a solution of KOH (130 g) in water (130 mL) at rt. The mixture was cooled to −15° C. (with an external MeOH-ice bath), and a solution of the (S)-2-((tert-butoxycarbonyl)amino)-3-hydroxypropanoic acid (61 g) and 2,4,6-trifluoronitrobenzene (52 g) in 2-MeTHF (400 mL) was added dropwise over 35 min maintaining the reaction temperature below 0° C. (during the last 75 mL of this addition, the internal temperature was at +3° C.). Following the addition, the reaction mixture was stirred at about −2° C. for 25 min. The cooling bath was switched to a dilute dry ice-acetone bath (−60° C.).

The reaction mixture was quenched by addition of 85% H₃PO₄ (185 mL) over 20 min maintaining the reaction temperature at −10 to 0° C. The mixture was stirred a few min at rt and filtered. The filtrate layers were separated, and the organic phase was dried over MgSO₄, filtered, and concentrated in vacuo (with a MeOH chase) to afford (S)-2-((tert-butoxycarbonyl)amino)-3-(3,5-difluoro-2-nitrophenoxy)propanoic acid of a pale yellow oil (130 g, 72% yield, 60% purity by UV). This material was used in the next step without further purification. MS ESI⁻ (m/z) 361.6 [M−H]⁻.

### Step 7: (S)-3-(2-Amino-3,5-difluorophenoxy)-2-(tert-butoxycarbonylamino)propanoic acid

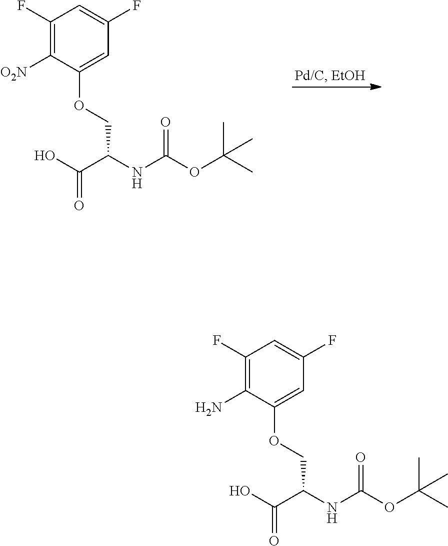 [SMILES: CC(C)(C)OC(=O)N[C@@H](COc1cc(F)cc(F)c1[N+](=O)[O-])C(=O)O ; CC(C)(C)OC(=O)N[C@@H](COc1cc(F)cc(F)c1N)C(=O)O]

To a solution of (S)-2-((tert-butoxycarbonyl)amino)-3-(3,5-difluoro-2-nitrophenoxy)propanoic acid (70 g, 193 mmol) in ethanol (400 mL) under nitrogen was added 10% Pd/C (12.34 g, 11.59 mmol). The reaction mixture was subjected to 50 psi hydrogen atmosphere at ambient temperature for 4 h. The reaction mixture was filtered through Celite® bed, washed with ethyl acetate. The combined filtrate was concentrated in vacuo to afford (S)-3-(2-amino-3,5-difluorophenoxy)-2-(tert-butoxycarbonylamino)propanoic acid (75 g, 80% yield, 68% purity by UV) as crude product. The crude product was used in the next step without further purification. MS ES⁺ m/z 333.27 [M+H]±.

### Step 8: (S)-tert-Butyl 6,8-difluoro-4-oxo-2,3,4,5-tetrahydrobenzo[b][1,4]oxazepin-3-ylcarbamate

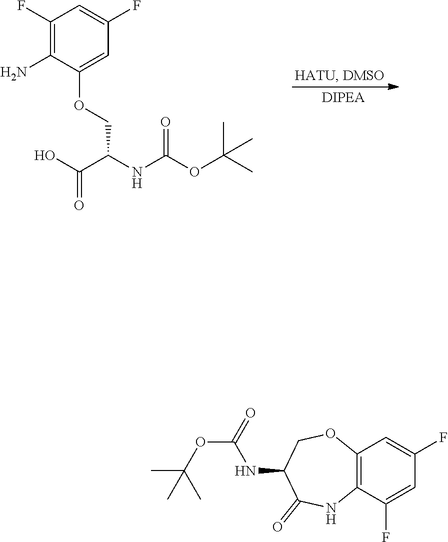 [SMILES: CC(C)(C)OC(=O)N[C@@H](COc1cc(F)cc(F)c1N)C(=O)O ; CC(C)(C)OC(=O)N[C@H]1COc2cc(F)cc(F)c2NC1=O]

To a stirred solution of (S)-3-(2-amino-3,5-difluorophenoxy)-2-((tertbutoxycarbonyl) amino)propanoic acid (90 g, 87 mmol) in DMSO (400 mL) was added HATU (45.2 g, 119 mmol) at rt. The reaction mixture was stirred at rt for 1.5 hours, and DIPEA (33.6 mL, 192 mmol) was added. The resulting mixture was stirred at rt for 4 hours and then poured into cold water to afford a precipitate. The solid material was collected by filtration. The solid material was purified by normal phase column chromatography (60-120 mesh silica gel; eluent: 15% EtOAc in petroleum ether). Collected fractions were concentrated in vacuo to afford brown color solid. Petroleum ether (150 mL) was added, and the mixture was stirred at rt for a few minutes and filtered to give (S)-tert-butyl (6,8-difluoro-4-oxo-2,3,4,5-tetrahydrobenzo[b][1,4]oxazepin-3-yl)carbamate as an off white solid (12 g, 37.9 mmol, 43.7% yield). MS ES⁺ m/z 315.13 [M+H]⁺; ¹H NMR (400 MHz, DMSO-d₆) δ 9.84 (s, 1H), 7.22 (t, J=10.4 Hz, 1H), 7.09 (d, J=7.20 Hz, 1H), 6.98 (d, J=9.60 Hz, 1H), 4.45-4.32 (m, 3H), 1.36 (s, 9H).

### Step 9: (S)-tert-Butyl 6,8-difluoro-5-methyl-4-oxo-2,3,4,5-tetrahydrobenzo[b][1,4]oxazepin-3-yl)carbamate

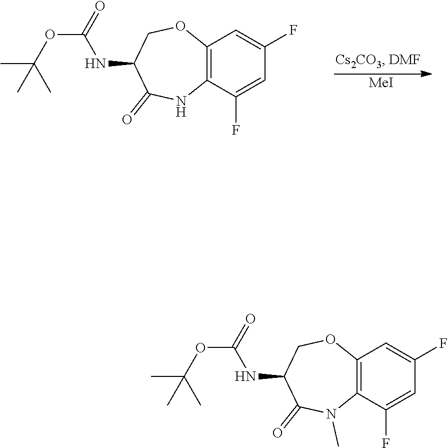 [SMILES: "MeI" ; CC(C)(C)OC(=O)N[C@H]1COc2cc(F)cc(F)c2NC1=O ; CN1C(=O)[C@@H](NC(=O)OC(C)(C)C)COc2cc(F)cc(F)c21]

To a solution of (S)-tert-butyl (6,8-difluoro-4-oxo-2,3,4,5-tetrahydrobenzo[b][1,4]oxazepin-3-yl)carbamate (6.10 g, 19.41 mmol) in DMF (75 mL) was added cesium carbonate (8.85 g, 27.2 mmol). The reaction mixture was stirred for 5 minutes and then iodomethane (1.396 mL, 22.32 mmol) was added. The mixture was stirred for 2 hours and then cooled in an ice-water bath. Water (100 mL) was added quickly dropwise, which resulted in gum-like solid. The material was diluted with water (200 mL) and diethyl ether. The organic layer was separated, washed with brine, concentrated, and dried to give (S)-tert-butyl (6,8-difluoro-5-methyl-4-oxo-2,3,4,5-tetrahydrobenzo[b][1,4]oxazepin-3-yl)carbamate as a pale pink-purple sticky foam (6.54 g, 98% yield). MS ES⁺ m/z 229.3 [M-Boc+H]⁺; ¹H NMR (400 MHz, DMSO-d₆) δ 7.37 (ddd, J=2.78, 9.16, 11.56 Hz, 1H), 7.21 (d, J=8.34 Hz, 1H), 7.03-7.12 (m, J=2.02, 9.09 Hz, 1H), 4.39-4.50 (m, J=8.08, 8.08 Hz, 1H), 4.29-4.36 (m, 2H), 3.17 (d, J=2.02 Hz, 3H), 1.35 (s, 9H).

### Step 10: (S)-3-Amino-6,8-difluoro-5-methyl-2,3-dihydrobenzo[b][1,4]oxazepin-4(5H)-one hydrochloride

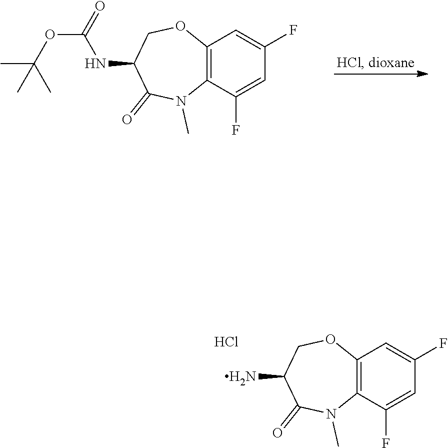

To a suspension of (S)-tert-butyl (6,8-difluoro-5-methyl-4-oxo-2,3,4,5-tetrahydrobenzo[b][1,4]oxazepin-3-yl)carbamate (25 g, 63.0 mmol) in DCM (150 mL) stirred under nitrogen at 0° C. was added 4.0 M HCl (250 ml, 1000 mmol) in dioxane. The reaction mixture was stirred at rt for 4 hours. The reaction mixture was cooled to −5° C. and filtered. The solid was washed with ether (100 mL) and dried to give (S)-3-amino-6,8-difluoro-5-methyl-2,3-dihydrobenzo[b][1,4]oxazepin-4(5H)-one hydrochloride as an off white solid (15 g, 56.6 mmol, 90% yield). MS ES⁺ m/z 228.88 [M+H]⁺; ¹H NMR (400 MHz, DMSO-d₆) δ 8.80 (s, 3H), 7.40 (t, J=8.8 Hz, 1H), 7.18 (d, J=8.8 Hz, 1H), 4.68-4.64 (m, 1H), 4.54-4.45 (m, 2H), 3.23 (d, J=2.0 Hz, 3H).

### Step 11: (5)-5-Benzyl-N-(6,8-difluoro-5-methyl-4-oxo-2,3,4,5-tetrahydrobenzo[b][1,4]oxazepin-3-yl)-4H-1,2,4-triazole-3-carboxamide

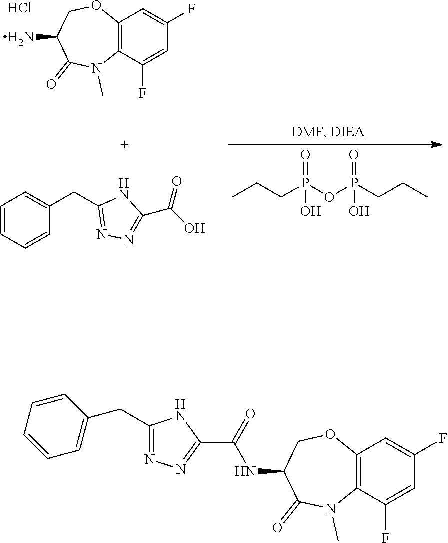

To a stirred solution of (S)-3-amino-6,8-difluoro-5-methyl-2,3-dihydrobenzo[b][1,4]oxazepin-4(5H)-one hydrochloride (15.0 g, 56.7 mmol, 1.0 eq) and 5-benzyl-4H-1,2,4-triazole-3-carboxylic acid (12.67 g, 62.3 mmol, 1.1 eq) in DMF (150 mL) was added DIEA (39.6 mL, 227 mmol, 5.0 eq) and propyl phosphonic anhydride (54.1 g, 85 mmol, 1.5 eq) in EtOAc (50%) dropwise over 15 min. The reaction mixture was stirred at ambient temperature for 2 h and then diluted with water (1000 mL). The resulting solid was filtered and dried. To the solid was added ethyl acetate (600 mL). The mixture was washed with saturated bicarbonate solution (300 mL) and brine (300 mL), dried over anhydrous sodium sulfate, and concentrated in vacuo to afford crude product (24.3 g). To the crude product was added EtOH (150 mL). The mixture was stirred at 80° C. for 10 min and then at rt for 2 days. The reaction mixture was filtered to collect crystalline solid. The solid was washed with cold EtOH (100 mL) and dried to afford (5)-5-benzyl-N-(6,8-difluoro-5-methyl-4-oxo-2,3,4,5-tetrahydrobenzo[b][1,4]oxazepin-3-yl)-4H-1,2,4-triazole-3-carboxamide as white crystalline solid (17.5 g, 74.5% yield). MS ES⁺ m/z 414.13 [M+H]⁺; ¹H NMR (400 MHz, DMSO-d₆) δ 14.33 (s, 1H), 8.41 (s br, 1H), 7.41-7.22 (m, 6H), 7.12 (d, J=8.8 Hz, 1H), 4.96-4.89 (m, 1H), 4.65 (s br, 1H), 4.44 (t, J=8.0 Hz, 1H), 4.12 (s, 2H), 3.20 (s, 3H).

### Example 5

### (S)-5-Benzyl-N-(7,9-difluoro-2-oxo-2,3,4,5-tetrahydro-1H-benzo[b]azepin-3-yl)-4H-1,2,4-triazole-3-carboxamide

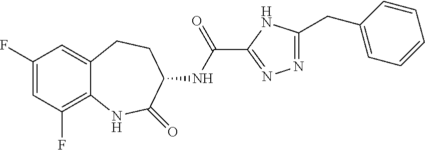 [SMILES: O=C(N[C@H]1CCc2cc(F)cc(F)c2NC1=O)c1nnc(Cc2ccccc2)[nH]1]

### Step 1: (E)-6,8-Difluoro-3,4-dihydronaphthalen-1(2H)-one oxime

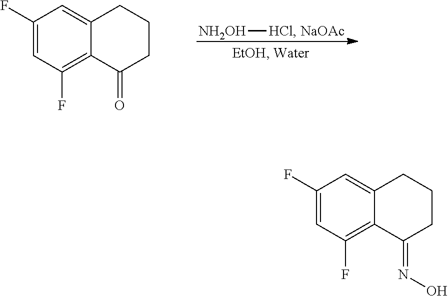

To a solution of 6,8-difluoro-3,4-dihydronaphthalen-1(2H)-one (50 g, 274 mmol) in ethanol (500 mL) and water (167 mL) was added sodium acetate (33.8 g, 412 mmol) and hydroxylamine hydrochloride (28.6 g, 412 mmol). The reaction turned from a light pink to light yellow after hydroxylamine hydrochloride was added and a precipitate formed after 5 minutes. The reaction was stirred at room temperature for 2 hrs 20 min. To the reaction mixture was added water (500 mL). The solids were filtered and rinsed with water. The solid was dried to give (E)-6,8-difluoro-3,4-dihydronaphthalen-1(2H)-one oxime as an off-white solid (51.2 g). On sitting for 18 hours, a small amount of additional solid had precipitated from the filtrate. This solid was also filtered, washed with water, and dried to give additional (E)-6,8-difluoro-3,4-dihydronaphthalen-1(2H)-one oxime (0.95 g). Total yield 52.15 g (96% yield). MS ES⁺ m/z 198 [M+H]⁺; ¹H NMR (400 MHz, DMSO-d₆) δ 11.33 (s, 1H), 7.09 (ddd, J=2.65, 9.35, 11.75 Hz, 1H), 7.00 (dd, J=1.39, 8.97 Hz, 1H), 2.61-2.77 (m, 4H), 1.71 (quin, J=6.38 Hz, 2H).

### Step 2: (E)-6,8-Difluoro-3,4-dihydronaphthalen-1(2H)-one 0-tosyl oxime

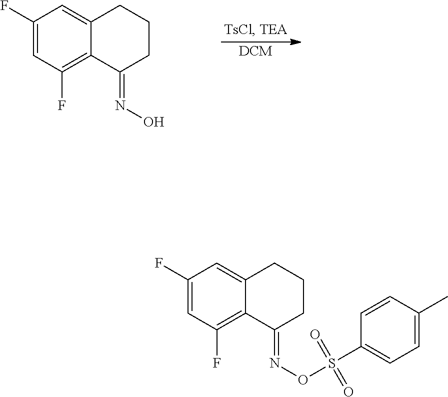 [SMILES: O/N=C1\CCCc2cc(F)cc(F)c21 ; Cc1ccc(S(=O)(=O)O/N=C2\CCCc3cc(F)cc(F)c32)cc1]

To a suspension of (E)-6,8-difluoro-3,4-dihydronaphthalen-1(2H)-one oxime (52.2 g, 265 mmol) in dichloromethane (600 mL) was added TEA (55.3 mL, 397 mmol). The reaction was cooled in an ice-water bath, and p-toluenesulfonyl chloride (53 g, 278 mmol) was added. The ice bath was removed, and the reaction mixture was stirred at room temperature for 22 hours. The reaction solution was washed with water (2×350 mL), 5% citric acid, and brine. The mixture was concentrated under reduced pressure and dried to afford (E)-6,8-difluoro-3,4-dihydronaphthalen-1(2H)-one 0-tosyl oxime as an orange-tan solid (92.1 g, 96% yield). MS ES⁺ m/z 352 [M+H]⁺; ¹H NMR (400 MHz, DMSO-d₆) δ 7.86 (d, J=8.34 Hz, 2H), 7.48 (d, J=8.08 Hz, 2H), 7.19 (ddd, J=2.53, 9.22, 11.49 Hz, 1H), 7.09 (dd, J=1.39, 8.97 Hz, 1H), 2.82 (t, J=6.57 Hz, 2H), 2.75 (t, J=6.06 Hz, 2H), 2.42 (s, 3H), 1.71 (quin, J=6.32 Hz, 2H).

### Step 3: 7,9-Difluoro-4,5-dihydro-1H-benzo[b]azepin-2(3H)-one

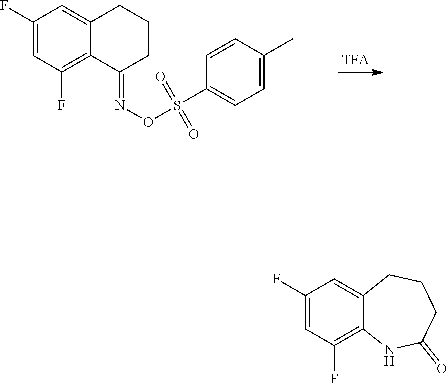 [SMILES: Cc1ccc(S(=O)(=O)O/N=C2\CCCc3cc(F)cc(F)c32)cc1 ; O=C1CCCc2cc(F)cc(F)c2N1]

To (E)-6,8-difluoro-3,4-dihydronaphthalen-1(2H)-one 0-tosyl oxime (92.1 g, 262 mmol) was added trifluoroacetic acid (220 mL). The reaction mixture was heated in an Opti Therm metal heating mantle at 50° C. and stirred for 10 minutes. The internal temperature was about 35° C. and the heating mantle temperature was raised to 65° C. After 5 minutes, the homogeneous reaction was dark brown and bubbled for about a minute and the internal temperature was about 70° C. After 10 minutes, the reaction was cooled to room temperature and further cooled in an ice-water bath. The reaction mixture was quenched with cold water (1000 mL) over 5 minutes. The reaction mixture was stirred vigorously for 30 minutes in an ice bath. The resulting precipitate was filtered and washed with water. The crude material was stirred in 10% diethyl ether/hexanes (500 mL), filtered, suspended in 25% diethyl ether/hexanes (500 mL), filtered, and suspended in diethyl ether (250 mL). The resulting solid was filtered and dried in a vacuum oven to give 7,9-difluoro-4,5-dihydro-1H-benzo[b]azepin-2(3H)-one (33.9 g, 60% yield) as a light brown solid. MS ES⁺ m/z 198 [M+H]⁺; ¹H NMR (400 MHz, DMSO-d₆) δ 9.40 (s, 1H), 7.20 (ddd, J=2.78, 9.16, 10.29 Hz, 1H), 7.06 (dd, J=1.52, 8.84 Hz, 1H), 2.73 (t, J=6.82 Hz, 2H), 2.05-2.20 (m, 4H).

### Step 4: 7,9-Difluoro-3-iodo-4,5-dihydro-1H-benzo[b]azepin-2(3H)-one

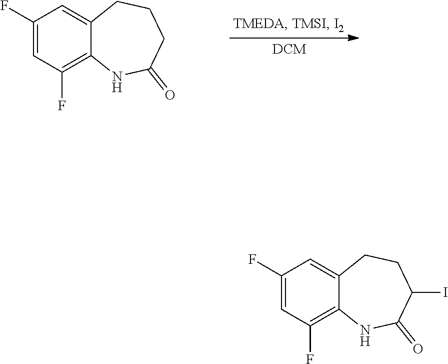 [SMILES: O=C1CCCc2cc(F)cc(F)c2N1 ; O=C1Nc2c(F)cc(F)cc2CCC1I]

To a mixture of 7,9-difluoro-4,5-dihydro-1H-benzo[b]azepin-2(3H)-one (33.9 g, 172 mmol) in dichloromethane (400 mL) cooled in an ice/water bath was added TMEDA (51.9 mL, 344 mmol), followed by TMSI (46.8 mL, 344 mmol) dropwise over 25 minutes. The light brown solution was stirred in the ice-bath for 60 minutes, and then iodine (65.4 g, 258 mmol) was added. The reaction mixture was stirred in the ice-bath for another 60 minutes, quenched with aq. sodium thiosulfate, and stirred for 15 minutes. The resulting solid was filtered, washed with water and dichloromethane, and dried under vacuum to give 7,9-difluoro-3-iodo-4,5-dihydro-1H-benzo[b]azepin-2(3H)-one (37.6 g, 66% yield) as a tan solid. The organic layer from the filtrate was separated and combined with dichloromethane washes. The combined organic layers were washed with water and brine, and concentrated under reduced pressure. The resulting solid was triturated in ethyl acetate (50 mL), filtered, and dried to give additional 7,9-difluoro-3-iodo-4,5-dihydro-1H-benzo[b]azepin-2(3H)-one as an off-white solid (15.7 g, 28% yield). MS ES⁺ m/z 324 [M+H]⁺; ¹H NMR (400 MHz, DMSO-d₆) δ 9.85 (s, 1H), 7.24 (dt, J=2.78, 9.60 Hz, 1H), 7.08 (dd, J=1.52, 8.84 Hz, 1H), 4.63-4.75 (m, 1H), 2.66-2.81 (m, 3H), 2.53-2.64 (m, 1H).

### Step 5: 3-Amino-7,9-difluoro-4,5-dihydro-1H-benzo[b]azepin-2(3H)-one

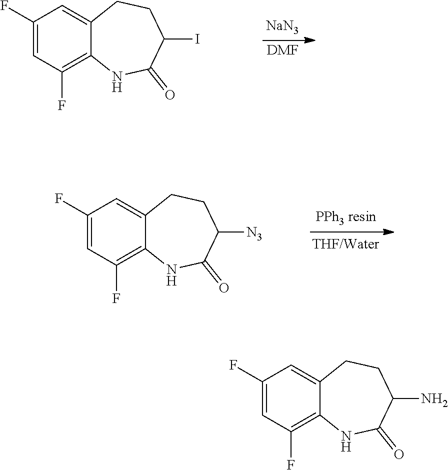

To a cloudy solution of 7,9-difluoro-3-iodo-4,5-dihydro-1H-benzo[b]azepin-2(3H)-one (53.2 g, 165 mmol) in N,N-dimethylformamide (400 mL) was added sodium azide (12.85 g, 198 mmol). The resulting was mixture stirred at room temperature for 45 minutes. To the reaction was added ice (300 mL) and then water (500 mL). The precipitation of a solid resulted. The reaction was stirred for 10 minutes and filtered to give 3-azido-7,9-difluoro-4,5-dihydro-1H-benzo[b]azepin-2(3H)-one as a tan solid. This was washed with water and used without further purification or drying. To a solution of 3-azido-7,9-difluoro-4,5-dihydro-1H-benzo[b]azepin-2(3H)-one (direct from previous step) in tetrahydrofuran (400 mL) was added water (2 mL) and PPh₃ resin (66 g, 3 mmol/g loading, 198 mmol). The reaction was stirred at room temperature for 24 h, filtered through a small celite plug to remove the resin, and rinsed with tetrahydrofuran. The filtrate was concentrated in vacuo. The resulting solid was triturated in Et₂O, filtered, and dried to give 3-amino-7,9-difluoro-4,5-dihydro-1H-benzo[b]azepin-2(3H)-one as an off-white solid (28.43 g, 80% yield over 2 steps). MS ES⁺ m/z 213 [M+H]⁺; ¹H NMR (400 MHz, DMSO-d₆) δ 9.59 (br s, 1H), 7.20 (ddd, J=2.78, 9.22, 10.23 Hz, 1H), 7.02-7.11 (m, 1H), 3.15 (dd, J=7.96, 11.49 Hz, 1H), 2.61-2.74 (m, 2H), 2.17-2.33 (m, 1H), 1.76 (dtd, J=2.78, 6.46, 17.91 Hz, 1H), 1.62 (br s, 2H).

### Step 6: (S)-3-Amino-7,9-difluoro-4,5-dihydro-1H-benzo[b]azepin-2(3H)-one

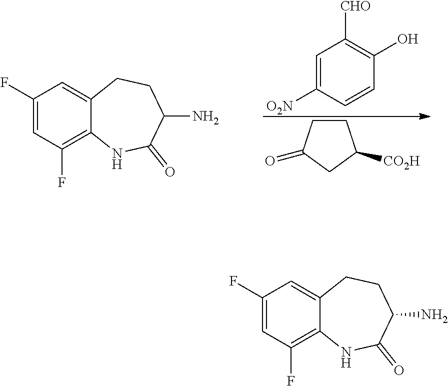

To a mechanically stirred solution of 3-amino-7,9-difluoro-4,5-dihydro-1H-benzo[b]azepin-2(3H)-one (28.4 g, 134 mmol) in isopropanol (1.25 L) at 70° C. was added 2-hydroxy-5-nitrobenzaldehyde (0.671 g, 4.02 mmol). Within 1 minute, a thick precipitate formed. L-pyroglutamic acid (17.28 g, 134 mmol) was added. The reaction mixture turned bright yellow and was stirred at 70° C. for 5 days and then cooled to −50° C. The solid was filtered and washed twice with isopropanol. The solid was suspended in hexanes, stirred, filtered, and dried to give (S)-3-amino-7,9-difluoro-4,5-dihydro-1H-benzo[b]azepin-2(3H)-one L-pyroglutamate salt as an off-white solid (37.97 g, % ee=94.7%). This material was suspended in 9:1 ACN:water (600 mL) and heated at 70° C. for 18 hours. The suspension was cooled to −40° C., filtered, washed with ACN, and dried to give (S)-3-amino-7,9-difluoro-4,5-dihydro-1H-benzo[b]azepin-2(3H)-one L-pyroglutamate salt as a white solid (35.8 g, % ee=100%). The salt was stirred vigorously in a mixture of 15 mL conc. ammonium hydroxide in 200 mL water for 7 minutes. The solid was filtered, re-suspended in a mixture of 15 mL conc. ammonium hydroxide in 200 mL water for 7 minutes, and filtered. The solid was stirred in water (200 mL) for 15 min, filtered, and dried to give (S)-3-amino-7,9-difluoro-4,5-dihydro-1H-benzo[b]azepin-2(3H)-one as a white solid (20.0 g, 70% yield). MS ES⁺ m/z 213 [M+H]⁺;

¹H NMR (400 MHz, DMSO-d₆) δ 9.59 (br. s., 1H), 7.15-7.29 (m, 1H), 7.07 (dd, J=1.52, 8.84 Hz, 1H), 3.15 (dd, J=7.83, 11.62 Hz, 1H), 2.59-2.77 (m, 2H), 2.16-2.31 (m, 1H), 1.69-1.83 (m, 1H), 1.60 (br s, 2H).

### Step 7: (S)-5-Benzyl-N-(7,9-difluoro-2-oxo-2,3,4,5-tetrahydro-1H-benzo[b]azepin-3-yl)-4H-1,2,4-triazole-3-carboxamide

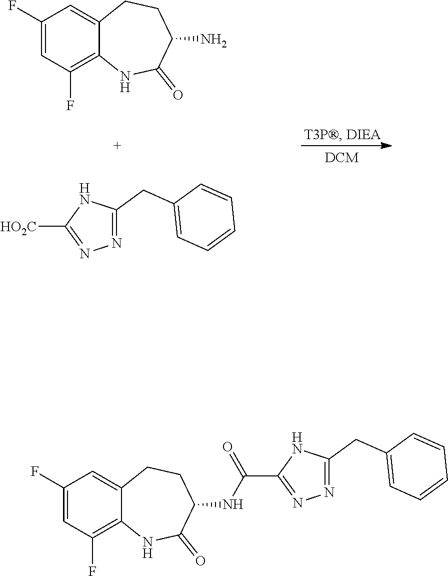 [SMILES: N[C@H]1CCc2cc(F)cc(F)c2NC1=O ; O=C(O)c1nnc(Cc2ccccc2)[nH]1 ; O=C(N[C@H]1CCc2cc(F)cc(F)c2NC1=O)c1nnc(Cc2ccccc2)[nH]1]

To a mixture of (S)-3-amino-7,9-difluoro-4,5-dihydro-1H-benzo[b]azepin-2(3H)-one (19.1 g, 90 mmol), 5-benzyl-4H-1,2,4-triazole-3-carboxylic acid (22.65 g, 95 mmol), and DIEA (47.2 mL, 270 mmol) in dichloromethane (650 mL) cooled in an ice-water bath was added a ≥50 wt. % solution of T3P in EtOAc (81 mL, 135 mmol) dropwise over 13 minutes. The mixture became more homogeneous during T3P addition. The ice bath was removed, and the reaction mixture was stirred at room temperature for 45 minutes becoming homogeneous after 10 minutes. The reaction was diluted with 0.5 M HCl (600 mL), and a solid precipitated from the organic phase. The 2 layers were separated. The organic phase, including the solid, were together treated with saturated sodium bicarbonate. The resulting organic and aqueous phases were shaken vigorously. The solid was filtered and washed with dichloromethane. The solid was stirred vigorously in water (600 mL) for 60 minutes, filtered, washed with water, and dried in a vacuum oven at 50° C. to give (S)-5-benzyl-N-(7,9-difluoro-2-oxo-2,3,4,5-tetrahydro-1H-benzo[b]azepin-3-yl)-4H-1,2,4-triazole-3-carboxamide as a white solid (33.1 g). The solid was re-suspended in water (700 mL) and stirred for 2 hours. The solid was filtered, washed with water, and dried in a vacuum oven at 50° C. to give (S)-5-benzyl-N-(7,9-difluoro-2-oxo-2,3,4,5-tetrahydro-1H-benzo[b]azepin-3-yl)-4H-1,2,4-triazole-3-carboxamide as a white solid (32.0 g, 88% yield). MS ES⁺ m/z 398 [M+H]⁺; ¹H NMR (DMSO-d₆) δ ppm ¹H NMR (400 MHz, DMSO-d₆) δ 13.98-15.03 (m, 1H), 9.96 (s, 1H), 7.90-8.85 (m, 1H), 7.20-7.39 (m, 6H), 7.15 (br d, J=8.84 Hz, 1H), 4.34 (td, J=7.89, 11.49 Hz, 1H), 4.01-4.20 (m, 2H), 2.69-2.91 (m, 2H), 2.14-2.48 (m, 2H).

### Example 6

### (S)-6-(4-(5-(3,5-difluorophenyl)-4,5-dihydro-1H-pyrazole-1-carbonyl)piperidin-1-yl)pyrimidine-4-carbonitrile

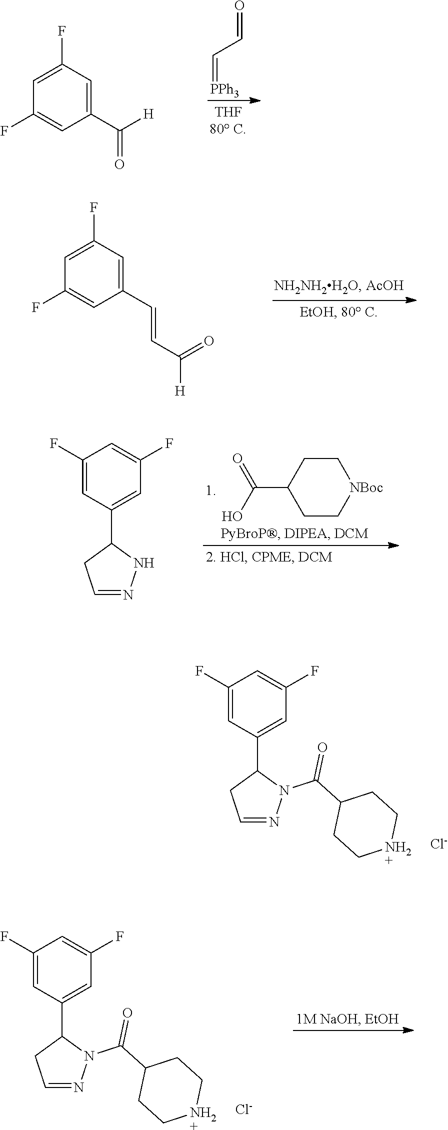

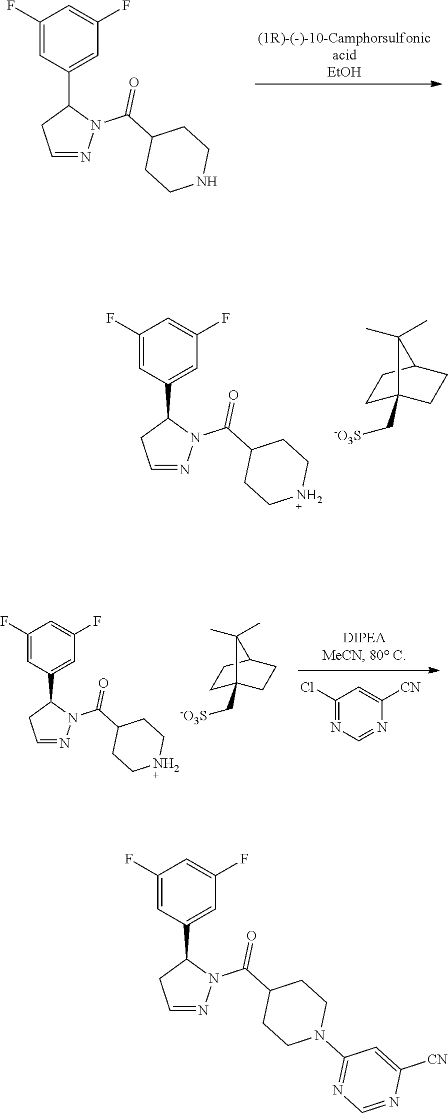

### Step 1

To a solution of 3,5-difluorobenzaldehyde (50 g, 352 mmol) in THF (300 mL) stirred under nitrogen at rt was added (triphenylphosphoranylidene)acetaldehyde (118 g, 387 mmol). The reaction mixture was stirred at 80° C. for 15 h and evaporated in vacuo. The residue was purified by normal phase column chromatography (CyH/EtOAc 100/0 to 90/10) to afford 3-(3,5-difluorophenyl)acrylaldehyde (25.6 g, 91 mmol, purity: 60%, recovery: 26%) as a yellow powder. LCMS (m/z) 169 (M+H)+, retention time: 2.28 min, LC/MS Method 1.

### Step 2

To a solution of hydrazine monohydrate (11.1 mL, 228 mmol) in ethanol (30 mL) was added acetic acid (14.8 mL, 259 mmol) at rt. The reaction mixture was heated to 45° C. and solid 3-(3,5-difluorophenyl)acrylaldehyde (25.6 g, 152 mmol) was added portion-wise during 20 min. The reaction vessel was sealed and heated to 80° C. for 21 h. The reaction mixture was concentrated in vacuo. The yellow residue was purified by normal phase column chromatography [CyH/(EtOAc/EtOH 3:1) 100/0 to 75/25] to afford 5-(3,5-difluorophenyl)-4,5-dihydro-1H-pyrazole (20 g, 110 mmol, purity: 63%, recovery: 72%) as an orange oil. LCMS (m/z) 183 (M+H)+, retention time: 1.89 min, LC/MS Method 1.

### Step 3

To a solution of 1-(tert-butoxycarbonyl)piperidine-4-carboxylic acid (25.2 g, 110 mmol) in DCM (300 mL) were added PyBroP® (53.7 g, 115 mmol) and DIPEA (21.09 mL, 121 mmol) at rt. After stirring for 5 min, 5-(3,5-difluorophenyl)-4,5-dihydro-1H-pyrazole (20 g, 110 mmol) was added. The reaction was stirred for 5 h and concentrated in vacuo. The residue was purified by normal phase column chromatography [CyH/(EtOAc/EtOH 3:1) 100/0 to 50/50] to provide tert-butyl 4-(5-(3,5-difluorophenyl)-4,5-dihydro-1H-pyrazole-1-carbonyl)piperidine-1-carboxylate, tert-Butyl 4-(5-(3,5-difluorophenyl)-4,5-dihydro-1H-pyrazole-1-carbonyl)piperidine-1-carboxylatewas dissolved in DCM (500 mL) and a 3 M solution of HCl in CPME (91 mL, 274 mmol) was added at rt. The reaction was stirred at rt for 24 h. The precipitate was filtered off, washed with DCM (2×150 mL) and iPr₂O (3×200 mL) to give (5-(3,5-difluorophenyl)-4,5-dihydro-1H-pyrazol-1-yl)(piperidin-4-yl)methanone, hydrochloride (20 g, 60.6 mmol, purity: 90%, recovery: 55%) as a cream powder. LCMS (m/z) 294 (M+H)+, retention time: 1.17 min, LC/MS Method 1.

### Step 4

To a solution of (5-(3,5-difluorophenyl)-4,5-dihydro-1H-pyrazol-1-yl)(piperidin-4-yl)methanone, hydrochloride (20 g, 60.6 mmol) in EtOH (50 mL) was added a 1 M solution of sodium hydroxide (79 mL, 79 mmol). The reaction mixture was stirred at rt for 30 min. DCM (150 mL) was added and the two layers were separated. The aqueous layer was extracted with DCM (2×150 mL). The combined organic layers were dried over sodium sulfate, filtered, and evaporated in vacuo to give the free base as an oil (17.3 g). This residue was dissolved in EtOH (50 mL) and (1R)-(−)-10-camphorsulfonic acid (14.09 g, 06.6 mmol) was added at rt. The resulting suspension was heated at 60° C. for 30 min. The solution was then evaporated to dryness and the partially crystalline crude solid was suspended and slurried in ethanol (50 mL) to fully convert it to a crystalline form, and this suspension was evaporated to dryness to give a light orange crystalline solid. This solid was recrystallized from EtOH (300 mL) to afford (S)-(5-(3,5-difluorophenyl)-4,5-dihydro-1H-pyrazol-1-yl)(piperidin-4-yl)methanone, 1R-(−)-camphor-10-sulphonic acid salt (7 g, 13.3 mmol, purity: 100%, recovery: 22%) as a white powder. LCMS (m/z) 294 (M+H)+, retention time: 1.17 min, LC/MS Method 1. Chiral HPLC Method 1: 2.58 and 3.26 min, % ee=99.2%.

### Step 5

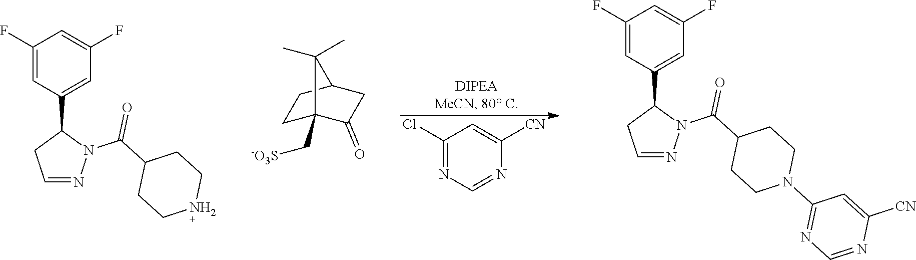

To a suspension of (S)-(5-(3,5-difluorophenyl)-4,5-dihydro-1H-pyrazol-1-yl)(piperidin-4-yl)methanone, 1R-(−)-camphor-10-sulphonic acid salt (300 mg, 0.57 mmol) in MeCN (30 mL) was added 6-chloropyrimidine-4-carbonitrile (80 mg, 0.57 mmol) and DIPEA (0.25 mL, 1.43 mmol) The vessel was sealed and heated at 80° C. for 2 h. The reaction mixture was evaporated in vacuo. This residue was purified by normal phase column chromatography [CyH/(EtOAc/EtOH 3:1) 100/0 to 70/30]. A trituration into iPr₂O afforded, after filtration, (S)-6-(4-(5-(3,5-difluorophenyl)-4,5-dihydro-1H-pyrazole-1-carbonyl)piperidin-1-yl)pyrimidine-4-carbonitrile (130 mg, 0.33 mmol, purity: 100%, recovery: 58%) as a light yellow powder. LCMS (m/z) 397 (M+H)+, retention time: 2.48 min, LC/MS Method 1. ¹H NMR (400 MHz, DMSO-d₆) δ ppm 8.54 (s, 1H), 7.57 (s, 1H), 7.26 (s, 1H), 7.12 (tt, J=9.4, 2.1 Hz, 1H), 6.84 (d, J=6.5 Hz, 2H), 5.34 (dd, J=12.0, 4.9 Hz, 1H), 4.47 (br.s, 2H), 3.49 (ddd, J=19.0, 12.0, 1.0 Hz, 1H), 3.43 (tt, J=11.4, 3.7 Hz, 1H), 3.13 (br s, 2H), 2.75 (ddd, J=19.2, 4.9, 1.5 Hz, 1H), 1.95 (d, J=12.7 Hz, 1H), 1.81 (d, J=12.7 Hz, 1H), 1.48 (m, 2H).

### Example 7

In vitro Assay: A fluorescent polarization based binding assay was developed to quantitate interaction of novel test compounds at the ATP binding pocket of RIP1, by competition with a fluorescently labeled ATP competitive ligand. Table 1 lists examples of pIC₅₀ data for the noted compounds of the Examples. The FP assay involves a fluorescent labeled ligand (14-(2-{[3-({2-{[4-(cyanomethyl)phenyl]amino}-6-[(5-cyclopropyl-1H-pyrazol-3-yl)amino]-4-pyrimidinyl}amino)propyl]amino}-2-oxoethyl)-16,16,18,18-tetramethyl-6,7,7a,8a,9,10,16,18-octahydrobenzo[2″,3″]indolizino[8″,7″:5′,6′]pyrano[3′,2′:3,4]pyrido[1,2-a]indol-5-ium-2-sulfonate at a final assay concentration of 5 nM. His-GST-RipK1(1-375) was purified from a Baculovirus expression system and was used at a final assay concentration of 10 nM. Both the enzyme and ligand were prepared in buffer consisting of 50 mM HEPES pH 7.5, 10 mM NaCl, 50 mM MgCl2, 0.5 mM DTT, and 0.02% CHAPS. Test compounds were prepared in neat DMSO and 100 nL was dispensed to individual wells of a multiwell plate. Next, 5 uL His-GST-RipK1(1-375) was added to the test compounds at twice the final assay concentration, and incubated at room temperature for 10 minutes. Following the incubation, 5 μL of the fluorescent labeled ligand solution, was added to each reaction, at twice the final assay concentration, and incubated at room temperature for at least 15 minutes. Finally, samples were read on an instrument capable of measuring fluorescent polarization. Test compound inhibition was expressed as percent inhibition of internal assay controls. For concentration response experiments, normalized data were fit and pIC₅₀ values determined using conventional techniques. Those of skill in the art will recognise that in vitro binding assays for functional activity are subject to experimental variability, accordingly, it is to be understood that the values given below are exemplary only. As determined using the above method, the compounds of Examples 1-5 exhibited a pIC₅₀ between approximately 5.0 and 9.0.

TABLE 1

| Example No. | FP Binding Assay (pIC₅₀) |
|---|---|
| 1 | 8.0 |
| 2 | 7.7 |
| 3 | 7.4 |
| 4 | 8.6 |
| 5 | 7.7 |
| 6 | 8.0 |

In vivo Assay: The efficacy of RIP1 inhibitors may be tested in mice in vivo using a TNF-driven systemic inflammatory response syndrome model (Duprez, L., et al., Immunity 35(6):908-918 (2011)). The model is run in a long modality (using TNF alone i.v.) which results in the termination of the study in ˜7-8 hrs (under IACUC guidelines for moribund endpoints) or a short modality (using TNF plus the caspase inhibitor zVAD i.v.) which is terminated at ˜2.5-3 hrs (under IACUC guidelines for moribund endpoints). TNF (or TNF/zVAD) induced manifestations include temperature loss, the production of numerous cytokines (including IL-6, IL-lb, MIP1β, and MIP2) in the periphery, liver and intestinal inflammation and an increase of markers of cellular (LDH and CK) and liver damage (AST and ALT) in the serum. Inhibition of these TNF (or TNF/zVAD) induced manifestations can be shown by orally or i.v. pre-dosing with selected compounds useful in this invention.

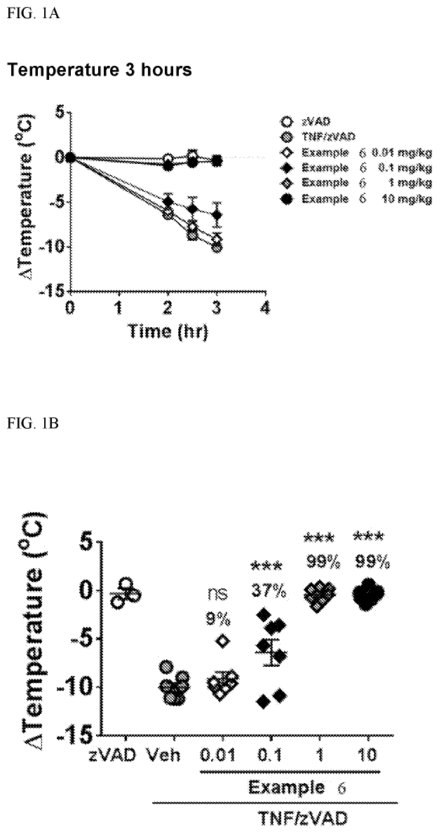

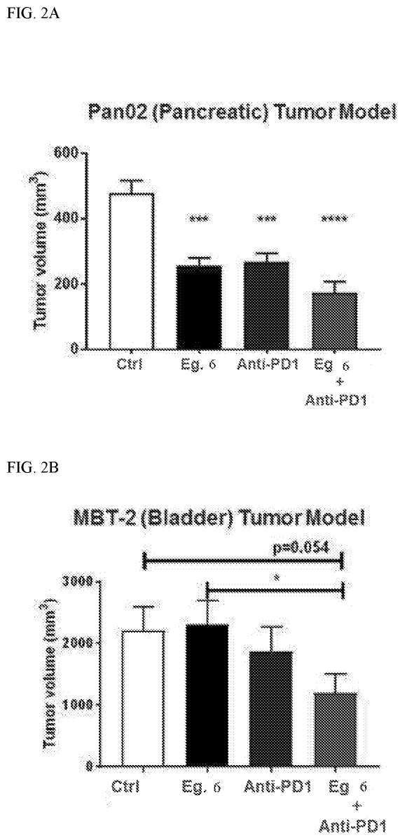

Each test compound is run through the TNF/zVAD version of the model. For example, mice (7 mice per group) are pre-dosed intravenously with vehicle or test compound 15 minutes before i.v. administration of mouse TNF (1.25 mg/kg/mouse) and zVAD (16.7 mg/kg/mouse) simultaneously. Temperature loss in mice is measured by rectal probe. The study is terminated when the control group became moribund, per our IACUC protocol. Representative data for the compound of Example 6 are provided in FIGS. 1A and 1B.

### Example 8

Subcutaneous Tumor Efficacy

The efficacy of RIP1 inhibition was tested in 12 different murine (6-8 week old) syngeneic subcutaneous tumor models. RIP1 inhibition was tested as a single agent in all models, with anti-PD1 combination arms added to the five of the final models.

TABLE 2
Study Design

| | | | Dose | Dosing | |
|---|---|---|---|---|---|
| Group | N | Treatment | (mg/kg) | Route | Schedule |
| 1 | 8 | PBS (saline) | 0 | i.p. | BIW × up to 21 days |
| 2 | 8 | Example 6 | 40 | p.o. | BID × up to 21 days |

Note:
N: animal number
Dosing volume: adjust dosing volume based on body weight (10 μl/g).
Treatment regimen may be changed per BW (body weight) loss.
The interval of BID dosing is 8 hours.

Study endpoints: The major endpoints of the study include the following:
1) Tumor growth inhibition (TGI): TGI % is an indication of antitumor effectiveness, and expressed as: TGI (%)=100×(1−T/C). T and C are the mean tumor volume of the treated and control groups, respectively, on a given day.
2) Tumor and plasma collection at study end for further investigation.

### Experimental Methods

Cell Culture: The 12 syngeneic cell lines were maintained in vitro with different medium (indicated in Table 3) at 37° C. in an atmosphere of 5% CO₂ in air. The tumor cells were routinely subcultured twice weekly. The cells in an exponential growth phase were harvested and counted for tumor inoculation.

TABLE 3
Medium information

| Cell line (tumor/carcinoma) | Medium |
|---|---|
| MBT2 (bladder) | RPMI1640 + 10% FBS |
| EMT-6 (breast) | DMEM + 10% FBS |
| CT26 (colon) | RPMI1640 + 10% FBS |
| MC38 (colon) | DMEM + 10% FBS |
| 22 (hepatoma) | RPMI1640 + 10% FBS |
| LL/2 (Lewis lung) | DMEM + 10% FBS |
| Renca (renal) | DMEM + 10% FBS |
| A20 (B lymphoma) | RPMI1640 + 10% FBS |
| B16F10 (melanoma) | DMEM + 10% FBS |
| B16BL6 (melanoma) | RPMI1640 + 10% FBS |
| Pan02 (pancreatic) | RPMI1640 + 10% FBS |
| RM-1(prostate) | RPMI1640 + 10% FBS |

Tumor Inoculation

Each mouse was inoculated subcutaneously with tumor cells in 0.1 mL of PBS for tumor development. The treatments were started when the mean tumor size reached approximately 80-120 mm³ (around 100 mm³). The test article (Example 6 or anti-PD1 (anti-mouse PD-1 antibody (clone RPM1-14), BioXcell) administration and the animal numbers in each study group are shown in the experimental design Table 2. The date of tumor cell inoculation is denoted as day 0.

Study Results

TABLE 4
Mean tumor growth inhibition (TGI) at model termination

| | | | RIP1i + |
|---|---|---|---|
| MuScreen Model | RIP1i TGI | Anti-PD1 TGI | Anti-PD1 TGI |
| EMT-6 | 21% | | |
| CT-26 | 19% | | |
| H-22 | 19% | | |
| LL/2 | 22% | | |
| Renca | 19% | | |
| A20 | 0.6% | | |
| B16F10 | 20% | | |
| Pan02 | PG21: 46% | PG21: 44% | PG21: 64% |
| MC38 | −16% | 46% | 62% |
| B16BL6 | 16% | −1.25% | 32% |
| RM-1 | −8% | −4% | 4% |
| MBT2 | −4% | 15.3% | 46% |

### Example 9

### Sharpin-Deficient Mouse Model of TNF-Dependent Dermatitis

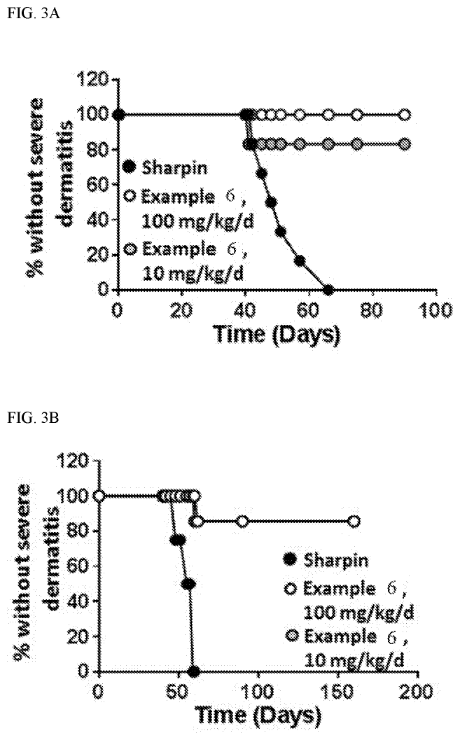

Sharpin-deficient mice (cpdm) develop a spontaneous and severe TNF- and RIPK1-dependent dermatitis and multi-organ immunopathology around 6-8 weeks of age (S. B. Berger et al., Journal of Immunology, 192(12):5476-5480, (2014)). Mice were dosed with a RIP1 inhibitor at the time of weaning (3-4 weeks of age) prior to the development of dermatitis lesions or therapeutically after the development of dermatitis lesions (about 6 weeks of age) using a food-based dosing regimen, such that mice (4-7 mice per group) received on average 100 mg/kg/day or 10 mg/kg/day of a RIP1 inhibitor in diet or control diet. Mice were observed for signs of proliferative dermatitis by using a dermatitis scoring system based on lesion character and regions affected. The character of the lesion was categorized according the following, in order of increasing severity, 0=none, 1=excoriation only or one small punctuated crust (<2 mm), 2=multiple, small punctuate crusts or coalescing crust (>2 mm), 3=erosion or ulceration. The regions were identified as: Region 1: the head cranial to the medial pinna attachment and/or lesions affecting the mandible cranial to the sternum, Region 2: inner and outer pinna, dorsal cervical region caudal to the medial pinna attachment, dorsal and ventral thorax, and thoracic limbs, Region 3: any region caudal to the ribcage. A score for the regions affected was categorized per the following: 0=none; 1=Region 2 or 3; 2=Region 2 and 3; 3=Region 1+/− other affected regions. To calculate the dermatitis severity score, the lesion score and regions affected score were summed, divided by 6, and then multiplied by 100. A severity score of 66 was considered severe dermatitis. Representative data for the compound of Example 6 are provided in FIGS. 3A and 3B.

### Example 10

### Subcutaneous Tumor Efficacy

Wild-type mice were implanted with orthotopic KPC-derived tumor cells and serially treated with a RIP1 inhibitor or fed control chow. Select cohorts were additionally treated with an agonizing ICOS mAb (E).

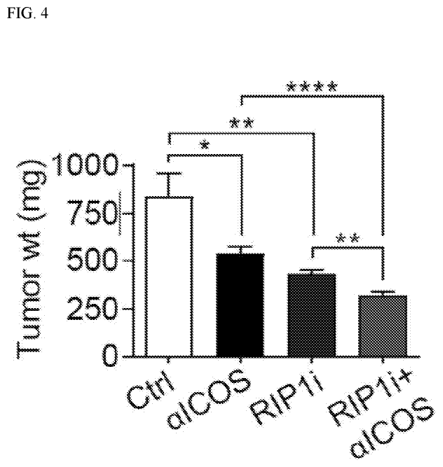

C57BL/6 mice were purchased from Jackson Labs (Bar Harbor, Me.) and bred in-house. Both male and female mice were used but animals were age-matched within each experiment. Mice were fed either control chow or a RIP1 inhibitor diet (˜100 mg/kg/day). For orthotopic pancreatic tumor challenge, 8-10 week old mice were administered intra-pancreatic injections of FC1242 PDA cells derived from KPC mice as described in Zambirinis, C. P. et al., J. Exp. Med. 212, 2077-2094, doi:10.1084/jem.20142162 (2015). Cells were suspended in PBS with 50% Matrigel (BD Biosciences, Franklin Lakes, N.J.) and 1×10⁵ tumor cells were injected into the body of the pancreas via laparotomy. Animals were treated i.p. with an agonistic ICOS mAb (7E.17G9, 100 μg, Days 4, 7 and 10 post-tumor challenge; BioXcell). Mice were sacrificed at 21 days (n=10/group) for analyses. Representative quantitative analyses of tumor weights using the compound of Example 6 are shown in FIG. 4.

### REFERENCES

Manguso, R. T. et al. Nature 547, 413-418 (2017)
Siefert, L. et al. Nature 532, 245-249 (2016)
Strilic, B. et al. Nature 536, 215-218 (2016)
Kondylis, V., et al. Cancer Cell 28, 582-598 (2015)

1. A combination comprising a RIP1 kinase inhibitor compound, or a salt, particularly a pharmaceutically acceptable salt thereof, and at least one other therapeutically active agent, wherein the at least one other therapeutically active agent is an immuno-modulator.

2. The combination of claim 1 wherein the RIP1 kinase inhibitor compound is a compound or a salt, particularly a pharmaceutically acceptable salt, thereof, of Formula (I): 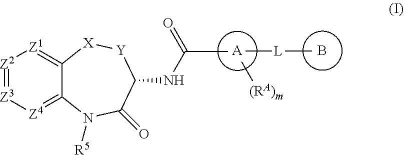
wherein:
X is CH₂;
Y is CH₂ or CH₂CH₂;
Z¹ is N, CH or CR¹;
Z² is CH or CR²;
Z³ is N, CH or CR³;
Z⁴ is CH or CR⁴;
R¹ is fluoro or methyl;
one of R² and R³ is halogen, cyano, (C₁-C₆)alkyl, halo(C₁-C₄)alkyl, (C₁-C₆)alkoxy, halo(C₁-C₄)alkoxy, hydroxyl, B(OH)₂, —COOH, halo(C₁-C₄)alkylC(OH)₂—, (C₁-C₄)alkoxy(C₁-C₄)alkoxy, (C₁-C₄)alkylSO₂—, (C₁-C₄)alkylSO₂NHC(O)—, (C₁-C₄)alkylC(O)NH—, ((C₁-C₄)alkyl)((C₁-C₄)alkyl)NC(O)—, (C₁-C₄)alkylOC(O)—, (C₁-C₄)alkylC(O)N(C₁-C₄)alkyl)-, (C₁-C₄)alkylNHC(O)—, (C₁-C₄)alkoxy(C₂-C₄)alkylNHC(O)—, (C₁-C₄)alkoxy(C₂-C₄)alkylC(O)NH—, (C₁-C₄)alkoxy(C₂-C₄)alkylNHC(O)NH—, (C₁-C₄)alkylSO₂(C₂-C₄)alkylNHC(O)—, (C₁-C₄)alkylNHC(O)NH—, (C₁-C₄)alkylOC(O)NH—, hydroxy(C₁-C₄)alkylOC(O)NH—, 5-6-membered heterocycloalkyl-C(O)—, 5-6-membered heterocycloalkyl-(C₁-C₄)alkyl-NHC(O)—, 5-6-membered heterocycloalkyl-(C₁-C₄)alkoxy-, 3-6-membered cycloalkyl, 5-6-membered heteroaryl, or 5-6-membered heteroaryl-C(O)NH,
wherein said 3-6-membered cycloalkyl, 5-6-membered heterocycloalkyl and 5-6-membered heteroaryl are optionally substituted by 1 or 2 substituents each independently selected from the group consisting of (C₁-C₄)alkyl and —(C₁-C₄)alkyl-CN;
and the other of R² and R³ is halogen, cyano or (C₁-C₆)alkyl;
R⁴ is fluoro, chloro, methyl or trifluoromethyl;
R⁵ is H or methyl;
A is phenyl, 5-6-membered heteroaryl, or 5-6-membered heterocycloalkyl, wherein the carbonyl moiety and L are substituted 1,3 on ring A;
m is 0 or m is 1 and R^{A} is (C₁-C₄)alkyl; and
L is O, S, NH, N(CH₃), CH₂, CH₂CH₂, CH(CH₃), CHF, CF₂, CH₂O, CH₂N(CH₃), CH₂NH, or CH(OH);
B is an optionally substituted (C₃-C₆)cycloalkyl, phenyl, 5-6-membered heteroaryl, or 5-6-membered heterocycloalkyl;
wherein said (C₃-C₆)cycloalkyl, phenyl, 5-6-membered heteroaryl, or 5-6-membered heterocycloalkyl is unsubstituted or is substituted by one or two substituents each independently selected from halogen, (C₁-C₄)alkyl, halo(C₁-C₄)alkyl, (C₁-C₄)alkoxy, halo(C₁-C₄)alkoxy, nitro, and (C₁-C₄)alkylC(O)—;
or the moiety -L-B is (C₃-C₆)alkyl, (C₃-C₆)alkoxy, halo(C₃-C₆)alkoxy, (C₃-C₆)alkenyl, or (C₃-C₆)alkenyloxy;
or Formula (II): 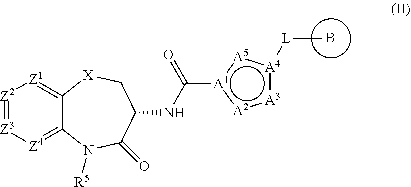
wherein:
X is CH₂;
Z¹ is CH;
Z² is CH or CR²;
Z³ is N, CH or CR³;
Z⁴ is CH or CR⁴;
R² and R⁴ are each independently selected from chloro or fluoro;
R⁵ is H or methyl;
L is CH₂;
A¹ and A⁴ are C, and A², A³, and A⁵ are each independently selected from N and NH to form a triazolyl ring moiety; and
B is a phenyl ring, optionally substituted by fluoro.

3. The combination of claim 1 wherein the RIP1 kinase inhibitor compound is: 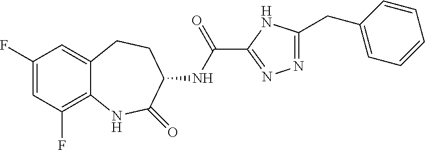 [SMILES: O=C(N[C@H]1CCc2cc(F)cc(F)c2NC1=O)c1nnc(Cc2ccccc2)[nH]1]
(S)-5-benzyl-N-(7,9-difluoro-2-oxo-2,3,4,5-tetrahydro-1H-benzo[b]azepin-3-yl)-4H-1,2,4-triazole-3-carboxamide, or a salt thereof, particularly a pharmaceutically acceptable salt, thereof.

4. The combination of claim 1, wherein said at least one immuno-modulator comprises at least one anti-CTLA4 antibody, anti-PD-1 antibody, anti-PD-L1 antibody, anti-OX-40 antibody or anti-ICOS antibody or antigen binding fragment thereof.

5. The combination of claim 1 wherein the immuno-modulator is selected from ipilimumab; tremelumumab; nivolumab; pembrolizumab; atezolizumab; durvalumumab; avelumab; at least one agonist antibody to human ICOS at least one agonist antibody to human OX-40.

6. The combination of claim 4 wherein the combination comprises a compound or a salt, particularly a pharmaceutically acceptable salt, thereof, of Formula (I): 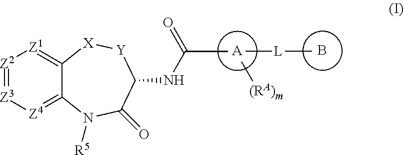
wherein:
X is CH₂;
Y is CH₂ or CH₂CH₂;
Z₁ is N, CH or CR¹;
Z² is CH or CR²;
Z³ is N, CH or CR³;
Z⁴ is CH or CR⁴;
R¹ is fluoro or methyl;
one of R² and R³ is halogen, cyano, (C₁-C₆)alkyl, halo(C₁-C₄)alkyl, (C₁-C₆)alkoxy, halo(C₁-C₄)alkoxy, hydroxyl, B(OH)₂, —COOH, halo(C₁-C₄)alkylC(OH)₂—, (C₁-C₄)alkoxy(C₁-C₄)alkoxy, (C₁-C₄)alkylSO₂—, (C₁-C₄)alkylSO₂NHC(O)—, (C₁-C₄)alkylC(O)NH—, ((C₁-C₄)alkyl)((C₁-C₄)alkyl)NC(O)—, (C₁-C₄)alkylOC(O)—, (C₁-C₄)alkylC(O)N(C₁-C₄)alkyl)-, (C₁-C₄)alkylNHC(O)—, (C₁-C₄)alkoxy(C₂-C₄)alkylNHC(O)—, (C₁-C₄)alkoxy(C₂-C₄)alkylC(O)NH—, (C₁-C₄)alkoxy(C₂-C₄)alkylNHC(O)NH—, (C₁-C₄)alkylSO₂(C₂-C₄)alkylNHC(O)—, (C₁-C₄)alkylNHC(O)NH—, (C₁-C₄)alkylOC(O)NH—, hydroxy(C₁-C₄)alkylOC(O)NH—, 5-6-membered heterocycloalkyl-C(O)—, 5-6-membered heterocycloalkyl-(C₁-C₄)alkyl-NHC(O)—, 5-6-membered heterocycloalkyl-(C₁-C₄)alkoxy-, 3-6-membered cycloalkyl, 5-6-membered heteroaryl, or 5-6-membered heteroaryl-C(O)NH,
wherein said 3-6-membered cycloalkyl, 5-6-membered heterocycloalkyl and 5-6-membered heteroaryl are optionally substituted by 1 or 2 substituents each independently selected from the group consisting of (C₁-C₄)alkyl and —(C₁-C₄)alkyl-CN;
and the other of R² and R³ is halogen, cyano or (C₁-C₆)alkyl;
R⁴ is fluoro, chloro, methyl or trifluoromethyl;
R⁵ is H or methyl;
A is phenyl, 5-6-membered heteroaryl, or 5-6-membered heterocycloalkyl, wherein the carbonyl moiety and L are substituted 1,3 on ring A;
m is 0 or m is 1 and R^{A} is (C₁-C₄)alkyl; and
L is O, S, NH, N(CH₃), CH₂, CH₂CH₂, CH(CH₃), CHF, CF₂, CH₂O, CH₂N(CH₃j, CH₂NH, or CH(OH);
B is an optionally substituted (C₃-C₆)cycloalkyl, phenyl, 5-6-membered heteroaryl, or 5-6-membered heterocycloalkyl;
wherein said (C₃-C₆)cycloalkyl, phenyl, 5-6-membered heteroaryl, or 5-6-membered heterocycloalkyl is unsubstituted or is substituted by one or two substituents each independently selected from halogen, (C₁-C₄)alkyl, halo(C₁-C₄)alkyl, (C₁-C₄)alkoxy, halo(C₁-C₄)alkoxy, nitro, and (C₁-C₄)alkylC(O)—;
or the moiety -L-B is (C₃-C₆)alkyl, (C₃-C₆)alkoxy, halo(C₃-C₆)alkoxy, (C₃-C₆)alkenyl, or (C₃-C₆)alkenyloxy;
or Formula (II): 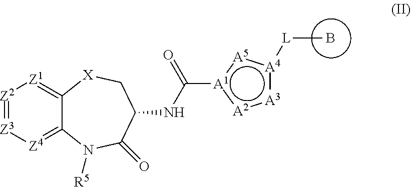
wherein:
X is CH₂;
Z¹ is CH;
Z² is CH or CR²;
Z³ is N, CH or CR³;
Z⁴ is CH or CR⁴;
R² and R⁴ are each independently selected from chloro or fluoro;
R⁵ is H or methyl;
L is CH₂;
A¹ and A⁴ are C, and A², A³, and A⁵ are each independently selected from N and NH to form a triazolyl ring moiety; and
B is a phenyl ring, optionally substituted by fluoro and an anti-PD-1 antibody selected from nivolumab and pembrolizumab.

7. The combination of claim 4 wherein the combination comprises a compound or a salt, particularly a pharmaceutically acceptable salt, thereof, of Formula (I): 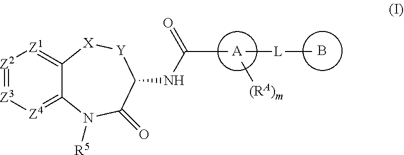
wherein:
X is CH₂;
Y is CH₂ or CH₂CH₂;
Z¹ is N, CH or CR¹;
Z² is CH or CR²;
Z³ is N, CH or CR³;
Z⁴ is CH or CR⁴;
R¹ is fluoro or methyl;
one of R² and R³ is halogen, cyano, (C₁-C₆)alkyl, halo(C₁-C₄)alkyl, (C₁-C₆)alkoxy, halo(C₁-C₄)alkoxy, hydroxyl, B(OH)₂, —COOH, halo(C₁-C₄)alkylC(OH)₂—, (C₁-C₄)alkoxy(C₁-C₄)alkoxy, (C₁-C₄)alkylSO₂—, (C₁-C₄)alkylSO₂NHC(O)—, (C₁-C₄)alkylC(O)NH—, ((C₁-C₄)alkyl)((C₁-C₄)alkyl)NC(O)—, (C₁-C₄)alkylOC(O)—, (C₁-C₄)alkylC(O)N(C₁-C₄)alkyl)-, (C₁-C₄)alkylNHC(O)—, (C₁-C₄)alkoxy(C₂-C₄)alkylNHC(O)—, (C₁-C₄)alkoxy(C₂-C₄)alkylC(O)NH—, (C₁-C₄)alkoxy(C₂-C₄)alkylNHC(O)NH—, (C₁-C₄)alkylSO₂(C₂-C₄)alkylNHC(O)—, (C₁-C₄)alkylNHC(O)NH—, (C₁-C₄)alkylOC(O)NH—, hydroxy(C₁-C₄)alkylOC(O)NH—, 5-6-membered heterocycloalkyl-C(O)—, 5-6-membered heterocycloalkyl-(C₁-C₄)alkyl-NHC(O)—, 5-6-membered heterocycloalkyl-(C₁-C₄)alkoxy-, 3-6-membered cycloalkyl, 5-6-membered heteroaryl, or 5-6-membered heteroaryl-C(O)NH,
wherein said 3-6-membered cycloalkyl, 5-6-membered heterocycloalkyl and 5-6-membered heteroaryl are optionally substituted by 1 or 2 substituents each independently selected from the group consisting of (C₁-C₄)alkyl and —(C₁-C₄)alkyl-CN;
and the other of R² and R³ is halogen, cyano or (C₁-C₆)alkyl;
R⁴ is fluoro, chloro, methyl or trifluoromethyl;
R⁵ is H or methyl;
A is phenyl, 5-6-membered heteroaryl, or 5-6-membered heterocycloalkyl, wherein the carbonyl moiety and L are substituted 1,3 on ring A;
m is 0 or m is 1 and R^{A} is (C₁-C₄)alkyl; and
L is O, S, NH, N(CH₃), CH₂—, CH₂CH, CH(CH₃), CHF, CF, CH₂O, CH₂N(CH₃), CH₂NH, or CH(OH);
B is an optionally substituted (C₃-C₆)cycloalkyl, phenyl, 5-6-membered heteroaryl, or 5-6-membered heterocycloalkyl;
wherein said (C₃-C₆)cycloalkyl, phenyl, 5-6-membered heteroaryl, or 5-6-membered heterocycloalkyl is unsubstituted or is substituted by one or two substituents each independently selected from halogen, (C₁-C₄)alkyl, halo(C₁-C₄)alkyl, (C₁-C₄)alkoxy, halo(C₁-C₄)alkoxy, nitro, and (C₁-C₄)alkylC(O)—;
or the moiety -L-B is (C₃-C₆)alkyl, (C₃-C₆)alkoxy, halo(C₃-C₆)alkoxy, (C₃-C₆)alkenyl, or (C₃-C₆)alkenyloxy;
or Formula (II): 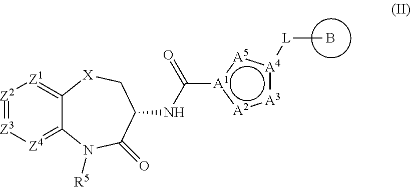
wherein:
X is CH₂;
Z¹ is CH;
Z² is CH or CR²;
Z³ is N, CH or CR³;
Z⁴ is CH or CR⁴;
R² and R⁴ are each independently selected from chloro or fluoro;
R⁵ is H or methyl;
L is CH₂;
A¹ and A⁴ are C, and A², A³, and A⁵ are each independently selected from N and NH to form a triazolyl ring moiety; and
B is a phenyl ring, optionally substituted by fluoro and an ICOS agonist antibody.

8. The combination of claim 4 wherein said combination comprises a compound or a salt, particularly a pharmaceutically acceptable salt, thereof, of Formula (I): 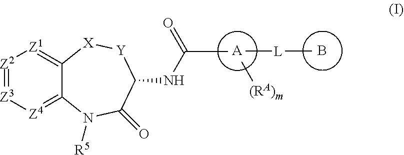
wherein:
X is CH,
Y is CH₂ or CH₂CH₂;
Z¹ is N, CH or CR¹;
Z² is CH or CR²;
Z³ is N, CH or CR³;
Z⁴ is CH or CR⁴;
R¹ is fluoro or methyl;
one of R² and R³ is halogen, cyano, (C₁-C₆)alkyl, halo(C₁-C₄)alkyl, (C₁-C₆)alkoxy, halo(C₁-C₄)alkoxy, hydroxyl, B(OH)₂—, —COOH, halo(C₁-C₄)alkylC(OH)₂—, (C₁-C₄)alkoxy(C₁-C₄)alkoxy, (C₁-C₄)alkylSO₂—, (C₁-C₄)alkylSO₂NHC(O)—, (C₁-C₄)alkylC(O)NH—, ((C₁-C₄)alkyl)((C₁-C₄)alkyl)NC(O)—, (C₁-C₄)alkylOC(O)—, (C₁-C₄)alkylC(O)N(C₁-C₄)alkyl)-, (C₁-C₄)alkylNHC(O)—, (C₁-C₄)alkoxy(C₂-C₄)alkylNHC(O)—, (C₁-C₄)alkoxy(C₂-C₄)alkylC(O)NH—, (C₁-C₄)alkoxy(C₂-C₄)alkylNHC(O)NH—, (C₁-C₄)alkylSO₂(C₂-C₄)alkylNHC(O)—, (C₁-C₄)alkylNHC(O)NH—, (C₁-C₄)alkylOC(O)NH—, hydroxy(C₁-C₄)alkylOC(O)NH—, 5-6-membered heterocycloalkyl-C(O)—, 5-6-membered heterocycloalkyl-(C₁-C₄)alkyl-NHC(O)—, 5-6-membered heterocycloalkyl-(C₁-C₄)alkoxy-, 3-6-membered cycloalkyl, 5-6-membered heteroaryl, or 5-6-membered heteroaryl-C(O)NH,
wherein said 3-6-membered cycloalkyl, 5-6-membered heterocycloalkyl and 5-6-membered heteroaryl are optionally substituted by 1 or 2 substituents each independently selected from the group consisting of (C₁-C₄)alkyl and —(C₁-C₄)alkyl-CN;
and the other of R² and R³ is halogen, cyano or (C₁-C₆)alkyl;
R⁴ is fluoro, chloro, methyl or trifluoromethyl;
R⁵ is H or methyl;
A is phenyl, 5-6-membered heteroaryl, or 5-6-membered heterocycloalkyl, wherein the carbonyl moiety and L are substituted 1,3 on ring A;
m is 0 or m is 1 and R^{A} is (C₁-C₄)alkyl; and
L is O, S, NH, N(CH₃), CH₂, CH₂CH₂, CH(CH₃), CHF, CF₂, CH₂O, CH₂N(CH₃), CH₂NH, or CH(OH);
B is an optionally substituted (C₃-C₆)cycloalkyl, phenyl, 5-6-membered heteroaryl, or 5-6-membered heterocycloalkyl;
wherein said (C₃-C₆)cycloalkyl, phenyl, 5-6-membered heteroaryl, or 5-6-membered heterocycloalkyl is unsubstituted or is substituted by one or two substituents each independently selected from halogen, (C₁-C₄)alkyl, halo(C₁-C₄)alkyl, (C₁-C₄)alkoxy, halo(C₁-C₄)alkoxy, nitro, and (C₁-C₄)alkylC(O)—;
or the moiety -L-B is (C₃-C₆)alkyl, (C₃-C₆)alkoxy, halo(C₃-C₆)alkoxy, (C₃-C₆)alkenyl, or (C₃-C₆)alkenyloxy;
or Formula 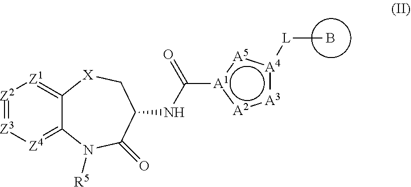
wherein:
X is CH₂;
Z¹ is CH;
Z² is CH or CR²;
Z³ is N, CH or CR³;
Z⁴ is CH or CR⁴;
R² and R⁴ are each independently selected from chloro or fluoro;
R⁵ is H or methyl;
L is CH₂;
A¹ and A⁴ are C, and A², A³, and A⁵ are each independently selected from N and NH to form a triazolyl ring moiety; and
B is a phenyl ring, optionally substituted by fluoro and an anti-ICOS antibody wherein the anti-ICOS antibody is an agonist antibody and wherein the anti-ICOS antibody comprises a V_{H} domain comprising an amino acid sequence at least 90% identical to the amino acid sequence set forth in SEQ ID NO:7 or a V_{L} domain comprising an amino acid sequence at least 90% identical to the amino acid sequence as set forth in SEQ ID NO:8 wherein said ICOS binding protein specifically binds to human ICOS.

9. The combination of claim 8 wherein the ICOS antibody comprises a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:7.

10. The combination of claim 8 wherein the ICOS antibody comprises a light chain variable region having about 85%, 86%, 87%, 88%, 89%, 90%, 91%, 92%, 93%, 94%, 95%, 96%, 97%, 98%, 99%, or 100% sequence identity to SEQ ID NO:8.

11. The combination of claim 8 wherein said combination comprises an ICOS antibody that binds to human ICOS with
(i) an association rate constant (kₒₙ) of at least 1×10⁵ M⁻¹s⁻¹; and a dissociation rate constant (k_{off}) of less than 6×10⁻⁵ s⁻¹; or
(ii) a dissociation constant (K_{D}) of less than about 100 nM,
wherein the affinity is measured by BIAcore.

12. A pharmaceutical composition comprising a combination according to claim 1 together with a pharmaceutically acceptable diluent or carrier.

13. (canceled)

14. (canceled)

15. A method of treating cancer in a human in need thereof comprising administering a therapeutically effective amount of the combination of claim 1.

16. The method of claim 15, wherein the cancer is a solid tumor.

17. The method of claim 15, wherein the cancer is selected from the group consisting of: pancreatic cancer, metastatic adenocarcinoma of the pancreas, pancreatic ductal adenocarcinoma, a malignancy of the endocrine cells in the pancreas, hepatocellular carcinoma, mesothelioma, melanoma, colorectal cancer, acute myeloid leukemia, metastasis, glioblastoma, breast cancer, gallbladder cancer, clear cell renal carcinoma, non-small cell lung carcinoma, and radiation induced necrosis.

18. The method of claim 15, wherein the cancer is Pancreatic ductal adenocarcinoma.

19. (canceled)

20. The method of treating cancer according to claim 15, wherein the combination comprises a RIP1 kinase inhibitor compound which is: 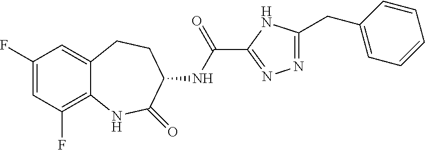 [SMILES: O=C(N[C@H]1CCc2cc(F)cc(F)c2NC1=O)c1nnc(Cc2ccccc2)[nH]1]
(S)-5-benzyl-N-(7,9-difluoro-2-oxo-2,3,4,5-tetrahydro-1H-benzo[b]azepin-3-yl)-4H-1,2,4-triazole-3-carboxamide or a tautomer thereof; or a pharmaceutically acceptable salt thereof,
wherein the cancer is selected from pancreatic cancer, metastatic adenocarcinoma of the pancreas, pancreatic ductal adenocarcinoma, and a malignancy of the endocrine cells in the pancreas, and
wherein the at least one immuno-modulator comprises at least one anti-CTLA4 antibody, anti-PD-1 antibody, anti-PD-L1 antibody, anti-OX-40 antibody or anti-ICOS antibody or an antigen binding fragment thereof.

21. A combination comprising a RIP1 kinase inhibitor compound, or a salt, particularly a pharmaceutically acceptable salt, thereof, which is: 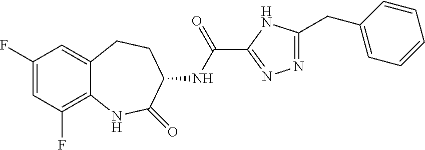 [SMILES: O=C(N[C@H]1CCc2cc(F)cc(F)c2NC1=O)c1nnc(Cc2ccccc2)[nH]1]
(S)-5-benzyl-N-(7,9-difluoro-2-oxo-2,3,4,5-tetrahydro-1H-benzo[b]azepin-3-yl)-4H-1,2,4-triazole-3-carboxamide, or a tautomer thereof, and at least one other therapeutically active agent, wherein the at least one other therapeutically active agent is an immuno-modulator.

22. A method of treating cancer in a human in need thereof comprising administering a therapeutically effective amount of combination comprising a RIP1 kinase inhibitor compound, or a salt, particularly a pharmaceutically acceptable salt, thereof, which is: 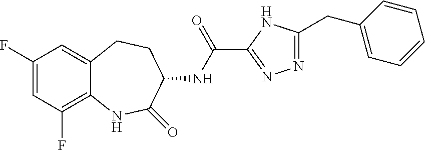 [SMILES: O=C(N[C@H]1CCc2cc(F)cc(F)c2NC1=O)c1nnc(Cc2ccccc2)[nH]1]
(S)-5-benzyl-N-(7,9-difluoro-2-oxo-2,3,4,5-tetrahydro-1H-benzo[b]azepin-3-yl)-4H-1,2,4-triazole-3-carboxamide and at least one other therapeutically active agent, or a tautomer thereof, wherein the at least one other therapeutically active agent is an immuno-modulator.

23. The method of claim 22, wherein the cancer is Pancreatic ductal adenocarcinoma.
